SEQUENCE LISTING (51 sequences)

SEQ ID NO: 1 - protein, 123 aa, Artificial Sequence
  Synthetic (7G7B6 VH)
EVQLVESGGDLVQPRGSLKLSCAASGFTFSSYGMSWVRQTPDKRLELVATINGYGDTTYYPDSVKGRFTISRDNAKNTLYLQMSSLKSEDTAMYFCARDRDYGNSYYYALDYWGQGTSVTVSS

SEQ ID NO: 2 - protein, 106 aa, Artificial Sequence
  Synthetic (7G7B6 VL)
QIVLSQSPAILSASPGERVTMTCRASSSVSFMHWLQQKPGSSPKPWIYATSNLASGVSARFSGSGSGTSYSLTITRVEAEDAATYYCQQWSSNPPAFGGGTKLEIK

SEQ ID NO: 3 - protein, 119 aa, Artificial Sequence
  Synthetic (MA251 VH)
QVQLKESGPGLVAPSQSLSITCTVSGFSLTSYGIQWVRQPPGKGLEWLGVIWAGGSTNYNSALMSRLSISKDNSKSQVFLKMNSLQTDDTAMYYCARAYGYDGSWLAYWGQGTLVTVSS

SEQ ID NO: 4 - protein, 106 aa, Artificial Sequence
  Synthetic (MA251 VL)
QIVLSQSPAILSASPGEKVTMTCRASSSVSYMHWYQQKPGSSPKPWIFATSNLASGVPARFSGSGSGTSYSLTINRVEAEDADTYYCQQWSSNPPTFGGGTKLEIK

SEQ ID NO: 5 - protein, 125 aa, Artificial Sequence
  Synthetic (Clone A VH)
EVQLVESGGGLIQPGGSLRLSCAASGFTLDSYGVSWVRQAPGKGLEWVGVTSSGGSAYYADSVKGRFTISRDNSKNTLYLQMNSLRAEDTAVYYCARDRYVYTGGYLYHYGMDLWGQGTLVTVSS

SEQ ID NO: 6 - protein, 109 aa, Artificial Sequence
  Synthetic (Clone A VL)
DIQMTQSPSSLSASVGDRVTITCRASQSISDYLAWYQQKPGKVPKLLIYAASTLPFGVPSRFSGSGSGTDFTLTISSLQPEDVATYYCQGTYDSSDWYWAFGGGTKVEI

SEQ ID NO: 7 - protein, 125 aa, Artificial Sequence
  Synthetic (Clone B VH)
EVQLLESGGGLVQPGGSLRLSCAASGFSVDIYDMSWVRQAPGKGLEWVAYISSSLGATYYADSVKGRFTISRDNSKNTLYLQMNSLRAEDTAVYYCARERIYSVYTLDYYAMDLWGQGTLVTVSS

SEQ ID NO: 8 - protein, 110 aa, Artificial Sequence
  Synthetic (Clone B VL)
DIQMTQSPSSLSASVGDRVTITCQASQGITNNLNWYQQKPGKVPKLLIYAASTLQSGVPSRFSGSGSGTDFTLTISSLQPEDVATYYCQQGYTTSNVDNAFGGGTKVEIK

SEQ ID NO: 9 - protein, 123 aa, Artificial Sequence
  Synthetic (Clone C, D VH)
EVQLLESGGGLVQPGGSLRLSCAASGFTFSSYGMSWVRQAPGKGLELVSTINGYGDTTYYPDSVKGRFTISRDNSKNTLYLQMNSLRAEDTAVYYCARDRDYGNSYYYALDYWGQGTLVTVSS

SEQ ID NO: 10 - protein, 123 aa, Artificial Sequence
  Synthetic (Clone E, F VH)
EVQLLESGGGLVQPGGSLRLSCAASGFTFSSYGMSWVRQAPGKGLELVSTINGYGDTTYYPDSVKGRFTISRDNAKNTLYLQMNSLRAEDTAVYFCARDRDYGNSYYYALDYWGQGTLVTVSS

SEQ ID NO: 11 - protein, 106 aa, Artificial Sequence
  Synthetic (Clone C, E VL)
EIVLTQSPGTLSLSPGERATLSCRASSSVSFMHWLQQKPGQAPRPLIYATSNLASGIPDRFSGSGSGTDYTLTISRLEPEDFAVYYCQQWSSNPPAFGQGTKLEIK

SEQ ID NO: 12 - protein, 106 aa, Artificial Sequence
  Synthetic (Clone D, F VL)
QIVLTQSPGTLSLSPGERATLSCRASSSVSFMHWLQQKPGQSPRPLIYATSNLASGIPDRFSGSGSGTDYTLTISRLEPEDFAVYYCQQWSSNPPAFGQGTKLEIK

SEQ ID NO: 13 - protein, 119 aa, Artificial Sequence
  Synthetic (Clone G, H, I, J VH)
QVQLQESGPGLVKPSETLSLTCTVSGFSLTSYGIQWIRQPPGKGLEWIGVIWAGGSTNYNSALMSRVTISKDNSKNQFSLKLSSVTAADTAVYYCARAYGYDGSWLAYWGQGTLVTVSS

SEQ ID NO: 14 - protein, 119 aa, Artificial Sequence
  Synthetic (Clone K, L, M, N VH)
QVQLQESGPGLVKPSETLSLTCTVSGFSLTSYGIQWVRQPPGKGLEWIGVIWAGGSTNYNSALMSRVTISKDNSKSQFSLKLSSVTAADTAVYYCARAYGYDGSWLAYWGQGTLVTVSS

SEQ ID NO: 15 - protein, 119 aa, Artificial Sequence
  Synthetic (Clone O, P, Q, R VH)
QVQLVESGGGVVQPGGSLRLSCAVSGFSLTSYGIQWVRQAPGKGLEWVSVIWAGGSTNYNSALMSRFTISKDNSKSTLYLQMNSLRAEDTAVYYCARAYGYDGSWLAYWGQGTLVTVSS

SEQ ID NO: 16 - protein, 106 aa, Artificial Sequence
  Synthetic (Clone G, K, O VL)
EIVLTQSPATLSLSPGERATLSCRASSSVSYMHWYQQKPGQAPRPLIFATSNLASGIPARFSGSGSGTDFTLTISSLEPEDFAVYYCQQWSSNPPTFGGGTKLEIK

SEQ ID NO: 17 - protein, 106 aa, Artificial Sequence
  Synthetic (Clone H, L, P VL)
QIVLTQSPATLSLSPGERATLSCRASSSVSYMHWYQQKPGQAPRPLIFATSNLASGIPARFSGSGSGTDYTLTISSLEPEDFAVYYCQQWSSNPPTFGGGTKLEIK

SEQ ID NO: 18 - protein, 106 aa, Artificial Sequence
  Synthetic (Clone I, M, Q VL)
DIQMTQSPSSLSASVGDRVTITCRASSSVSYMHWYQQKPGKAPKPLIFATSNLASGVPSRFSGSGSGTDYTLTISSLQPEDFATYYCQQWSSNPPTFGGGTKLEIK

SEQ ID NO: 19 - protein, 106 aa, Artificial Sequence
  Synthetic (Clone J, N, R VL)
QIQLTQSPSSLSASVGDRVTITCRASSSVSYMHWYQQKPGKSPKPLIFATSNLASGVPSRFSGSGSGTDYTLTISSLQPEDFATYYCQQWSSNPPTFGGGTKLEIK

SEQ ID NO: 20 - protein, 7 aa, Artificial Sequence
  Synthetic (CDRH1)
GFTFSSY

SEQ ID NO: 21 - protein, 6 aa, Artificial Sequence
  Synthetic (CDRH2)
NGYGDT

SEQ ID NO: 22 - protein, 14 aa, Artificial Sequence
  Synthetic (CDRH3)
DRDYGNSYYYALDY

SEQ ID NO: 23 - protein, 10 aa, Artificial Sequence
  Synthetic (CDRL1)
RASSSVSFMH

SEQ ID NO: 24 - protein, 7 aa, Artificial Sequence
  Synthetic (CDRL2)
ATSNLAS

SEQ ID NO: 25 - protein, 9 aa, Artificial Sequence
  Synthetic (CDRL3)
QQWSSNPPA

SEQ ID NO: 26 - protein, 7 aa, Artificial Sequence
  Synthetic (CDRH1)
GFSLTSY

SEQ ID NO: 27 - protein, 5 aa, Artificial Sequence
  Synthetic (CDRH2)
WAGGS

SEQ ID NO: 28 - protein, 11 aa, Artificial Sequence
  Synthetic (CDRH3)
AYGYDGSWLAY

SEQ ID NO: 29 - protein, 10 aa, Artificial Sequence
  Synthetic (CDRL1)
RASSSVSYMH

SEQ ID NO: 30 - protein, 9 aa, Artificial Sequence
  Synthetic (CDRL3)
QQWSSNPPT

SEQ ID NO: 31 - protein, 7 aa, Artificial Sequence
  Synthetic (Clone A CDRH1)
GFTLDSY

SEQ ID NO: 32 - protein, 5 aa, Artificial Sequence
  Synthetic (Clone A CDRH2)
SSGGS

SEQ ID NO: 33 - protein, 17 aa, Artificial Sequence
  Synthetic (Clone A CDRH3)
DRYVYTGGYLYHYGMDL

SEQ ID NO: 34 - protein, 11 aa, Artificial Sequence
  Synthetic (Clone A CDRL1)
RASQSISDYLA

SEQ ID NO: 35 - protein, 7 aa, Artificial Sequence
  Synthetic (Clone A CDRL2)
AASTLPF

SEQ ID NO: 36 - protein, 12 aa, Artificial Sequence
  Synthetic (Clone A CDRL3)
QGTYDSSDWYWA

SEQ ID NO: 37 - protein, 7 aa, Artificial Sequence
  Synthetic (Clone B CDRH1)
GFSVDIY

SEQ ID NO: 38 - protein, 6 aa, Artificial Sequence
  Synthetic (Clone B CDRH2)
SSSLGA

SEQ ID NO: 39 - protein, 16 aa, Artificial Sequence
  Synthetic (Clone B CDRH3)
ERIYSVYTLDYYAMDL

SEQ ID NO: 40 - protein, 11 aa, Artificial Sequence
  Synthetic (Clone B CDRL1)
QASQGITNNLN

SEQ ID NO: 41 - protein, 7 aa, Artificial Sequence
  Synthetic (Clone B CDRL2)
AASTLQS

SEQ ID NO: 42 - protein, 12 aa, Artificial Sequence
  Synthetic (Clone B CDRL3)
QQGYTTSNVDNA

SEQ ID NO: 43 - protein, 114 aa, Artificial Sequence
  Synthetic (7D4 VH)
EVQLQQSGAALVKPGASVKMSCKASGYSFPDSWVTWVKQSHGKSLEWIGDIFPNSGATNFNEKFKGKATLTVDKSTSTAYMELSRLTSEDSAIYYCTRLDYGYWGQGVMVTVSS

SEQ ID NO: 44 - protein, 324 aa, Artificial Sequence
  Synthetic (7D4-mIgG1 CH)
AKTTPPSVYPLAPGSAAQTNSMVTLGCLVKGYFPEPVTVTWNSGSLSSGVHTFPAVLQSDLYTLSSSVTVPSSTWPSQTVTCNVAHPASSTKVDKKIVPRDCGCKPCICTVPEVSSVFIFPPKPKDVLMISLTPKVTCVVVDISKDDPEVQFSWFVDDVEVHTAQTKPREEQINSTFRSVSELPILHQDWLNGKEFKCRVNSAAFPAPIEKTISKTKGRPKAPQVYTIPPPKEQMAKDKVSLTCMITNFFPEDITVEWQWNGQPAENYKNTQPIMDTDGSYFVYSKLNVQKSNWEAGNTFTCSVLHEGLHNHHTEKSLSHSPGK

SEQ ID NO: 45 - protein, 330 aa, Artificial Sequence
  Synthetic (7D4-mIgG2a CH)
AKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 46 - protein, 112 aa, Artificial Sequence
  Synthetic (7D4 VL)
DVVLTQTPPTLSATIGQSVSISCRSSQSLLHSNGNTYLNWLLQRPGQPPQLLIYLASRLESGVPNRFSGSGSGTDFTLKISGVEAEDLGVYYCVQSSHFPNTFGVGTKLEIK

SEQ ID NO: 47 - protein, 107 aa, Artificial Sequence
  Synthetic (7D4-mIgG1 CL, 7D4-mIgG2a CL)
RADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 48 - protein, 119 aa, Artificial Sequence
  Synthetic (PC61 VH)
EVQLQQSGAELVRPGTSVKLSCKVSGDTITAYYIHFVKQRPGQGLEWIGRIDPEDDSTEYAEKFKNKATITANTSSNTAHLKYSRLTSEDTATYFCTTDNMGATEFVYWGQGTLVTVSS

SEQ ID NO: 49 - protein, 322 aa, Artificial Sequence
  Synthetic (PC61-mIgG1 CH)
AKTTPPSVYPLAPGSAAQTNSMVTLGCLVKGYFPEPVTVTWNSGSLSSGVHTFPAVLESDLYTLSSSVTVPSSPRPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMNTNGSYFVYSKLNVQKSNWEAGNTFTCSVLHEGLHNHHTEKSLSHSP

SEQ ID NO: 50 - protein, 110 aa, Artificial Sequence
  Synthetic (PC61 VL)
QVVLTQPKSVSASLESTVKLSCKLNSGNIGSYYMHWYQQREGRSPTNLIYRDDKRPDGAPDRFSGSIDISSNSAFLTINNVQTEDEAMYFCHSYDGRMYIFGGGTKLTVL

SEQ ID NO: 51 - protein, 106 aa, Artificial Sequence
  Synthetic (PC61-mIgG1 CL)
GQPKSSPSVTLFPPSSEELETNKATLVCTITDFYPGVVTVDWKVDGTPVTQGMETTQPSKQSNNKYMASSYLTLTARAWERHSSYSCQVTHEGHTVEKSLSRADCS

(19) United States
(12) Patent Application Publication
(10) Pub. No.: US 2024/0307541 A1
(43) Pub. Date: Sep. 19, 2024

(54) METHODS FOR LOCAL AND SYSTEMIC TREATMENT OF CANCERS, TUMORS AND TUMOR CELLS

(71) Applicant: Rakuten Medical, Inc., San Diego, CA (US)

(72) Inventors: Nikolai SUSLOV, Needham, MA (US); Miguel GARCIA-GUZMAN, San Diego, CA (US); Jerry FONG, San Diego, CA (US); Robert J. HOEY, San Diego, CA (US); Jack BUI, San Diego, CA (US)

(73) Assignee: Rakuten Medical, Inc., San Diego, CA (US)

(21) Appl. No.: 18/274,985

(22) PCT Filed: Feb. 1, 2022
(86) PCT No.: PCT/US2022/014804

§ 371 Date: Jul. 28, 2023

Related U.S. Application Data
(60) Provisional application No. 63/144,931, filed on Feb. 2, 2021.

(51) Int. Cl. A61K 41/00; A61K 47/68; A61P 35/00; C07K 16/28

(52) U.S. Cl. CPC A61K 41/0071; A61K 47/6803; A61K 47/6849; A61P 35/00; C07K 16/2818

(57) ABSTRACT

Provided are conjugates, compositions, methods and uses related to treating a subject having a cancer, such as a cancer comprising a first tumor, a primary tumor, metastatic tumor cells, and/or invasive tumor cells. The methods include administering to the subject a targeting molecule that binds interleukin-2 receptor alpha chain (CD25) without substantially blocking or interfering with IL-2 signaling, conjugated with phthalocyanine dye, such as IR700, followed by illuminating a first tumor or primary tumor with a wavelength of light to activate the phthalocyanine dye. The methods and uses described herein provide for reduction, growth and elimination of tumors and tumor cells including first tumors, primary tumors, metastatic tumor cells, and/or invasive tumor cells. Also provided are methods and uses for enhancing systemic immunity against tumor growth in a subject having a cancer, a tumor or a lesion.

### CROSS-REFERENCE TO RELATED APPLICATIONS

This application claims priority from U.S. Provisional Application No. 63/144,931, filed Feb. 2, 2021, entitled “METHODS FOR LOCAL AND SYSTEMIC TREATMENT OF CANCERS, TUMORS AND TUMOR CELLS,” the contents of which are incorporated by reference in their entirety.

### INCORPORATION BY REFERENCE OF SEQUENCE LISTING

The present application is being filed along with a Sequence Listing in electronic format. The Sequence Listing is provided as a file entitled 751702002240SeqList.txt, created Jan. 31, 2022, which is 38.2 kilobytes in size. The information in the electronic format of the Sequence Listing is incorporated by reference in its entirety.

### FIELD

The present disclosure relates to conjugates, compositions, methods and uses related to treating a subject having a cancer, such as a cancer comprising a first tumor, a primary tumor, metastatic tumor cells, and/or invasive tumor cells. The methods include administering to the subject a targeting molecule that binds interleukin-2 receptor alpha chain (CD25) without substantially blocking or interfering with IL-2 signaling, conjugated with phthalocyanine dye, such as IR700, followed by illuminating a first tumor or primary tumor with a wavelength of light to activate the phthalocyanine dye. The methods and uses described herein provide for reduction, growth and elimination of tumors and tumor cells including first tumors, primary tumors, metastatic tumor cells, and/or invasive tumor cells. Also provided are methods and uses for enhancing systemic immunity against tumor growth in a subject having a cancer, a tumor or a lesion.

### BACKGROUND

Cancer metastasis is the primary cause of cancer-related deaths. Despite some available treatments for certain types of cancers, therapeutic strategies are still urgently needed for effectively treating cancers, including those that involve both primary and metastatic tumors. Treating metastatic cancers, in particular, still represents a great clinical challenge.

### SUMMARY

Provided herein are compositions, combinations, methods and uses for treating cancer. In some of any embodiments, provided herein is a conjugate including an antibody or antigen-binding fragment that specifically binds a CD25 without substantially blocking or interfering with IL-2 signaling, and a Si-phthalocyanine dye. In some of any embodiments, the conjugate is activated by illumination at a wavelength between at or about 600 nm and at or about 850 nm to effect cell killing. In some of any embodiments, the activated conjugate does not substantially block or interfere with IL-2 signaling. In some of any embodiments, the Si-phthalocyanine dye is IR700. In some of any embodiments, the Si-phthalocyanine dye has the structure of Formula (I), or a salt, stereoisomer, or tautomer thereof:

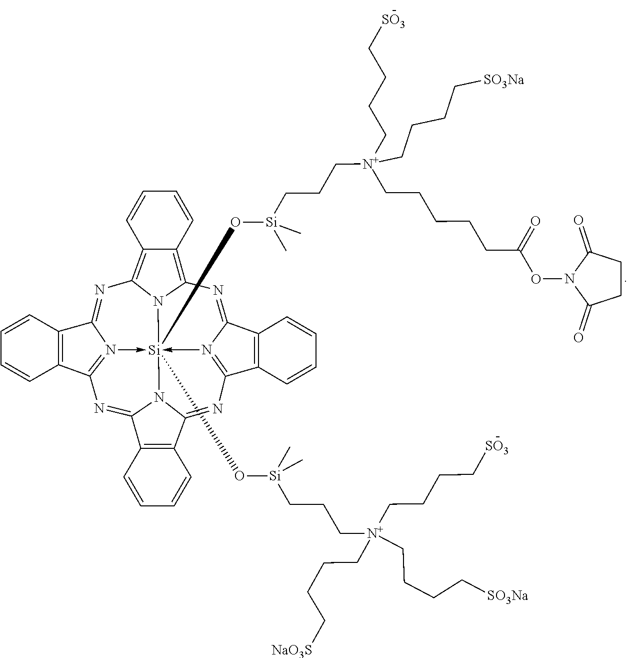

In some of any embodiments, the activated conjugate effects tumor inhibition or killing at a higher level, activity or potency than the unconjugated antibody.

Provided herein are conjugates comprising an antibody or antigen-binding fragment that specifically binds a CD25 without substantially blocking or interfering with IL-2 signaling, and a Si-phthalocyanine dye; wherein the conjugate is activated by illumination at a wavelength between at or about 600 nm and at or about 850 nm to effect cell killing.

In some of any embodiments, the antibody or antigen-binding fragment includes a heavy chain variable (V_{H}) region and a light chain variable (V_{L}) region. In some of any embodiments, the V_{H} region includes a heavy chain complementarity determining region 1 (CDR-H1), a heavy chain complementarity determining region 2 (CDR-H2) and a heavy chain complementarity determining region 3 (CDR-H3) contained within the V_{H} region amino acid sequence set forth in SEQ ID NO: 1 and the V_{L} region includes a light chain complementarity determining region 1 (CDR-L1), a light chain complementarity determining region 2 (CDR-L2) and a light chain complementarity determining region 3 (CDR-L3) contained within the V_{L} region amino acid sequence set forth in SEQ ID NO: 2. In some of any embodiments, the V_{H} region includes a CDR-H1, a CDR-H2 and a CDR-H3 contained within the V_{H} region amino acid sequence set forth in SEQ ID NO: 3 and the V_{L} region includes a CDR-L1, a CDR-L2 and a CDR-L3 contained within the V_{L} region amino acid sequence set forth in SEQ ID NO: 4. In some of any embodiments, the V_{H} region includes a CDR-H1, a CDR-H2 and a CDR-H3 contained within the V_{H} region amino acid sequence set forth in SEQ ID NO: 5 and the V_{L} region includes a CDR-L1, a CDR-L2 and a CDR-L3 contained within the V_{L} region amino acid sequence set forth in SEQ ID NO: 6.

In some of any embodiments, the V_{H} region includes a CDR-H1, a CDR-H2 and a CDR-H3 contained within the V_{H} region amino acid sequence set forth in SEQ ID NO: 7 and the V_{L} region includes a CDR-L1, a CDR-L2 and a CDR-L3 contained within the V_{L} region amino acid sequence set forth in SEQ ID NO: 8. In some of any embodiments, the V_{H} region includes a CDR-H1, a CDR-H2 and a CDR-H3 contained within the V_{H} region amino acid sequence set forth in SEQ ID NO: 9 and the V_{L} region includes a CDR-L1, a CDR-L2 and a CDR-L3 contained within the V_{L} region amino acid sequence set forth in SEQ ID NO: 11. In some of any embodiments, the V_{H} region includes a CDR-H1, a CDR-H2 and a CDR-H3 contained within the V_{H} region amino acid sequence set forth in SEQ ID NO: 9 and the V_{L} region includes a CDR-L1, a CDR-L2 and a CDR-L3 contained within the V_{L} region amino acid sequence set forth in SEQ ID NO: 12. In some of any embodiments, the V_{H} region includes a CDR-H1, a CDR-H2 and a CDR-H3 contained within the V_{H} region amino acid sequence set forth in SEQ ID NO: 10 and the V_{L} region includes a CDR-L1, a CDR-L2 and a CDR-L3 contained within the V_{L} region amino acid sequence set forth in SEQ ID NO: 11. In some of any embodiments, the V_{H} region includes a CDR-H1, a CDR-H2 and a CDR-H3 contained within the V_{H} region amino acid sequence set forth in SEQ ID NO: 10 and the V_{L} region includes a CDR-L1, a CDR-L2 and a CDR-L3 contained within the V_{L} region amino acid sequence set forth in SEQ ID NO: 12. In some of any embodiments, the V_{H} region includes a CDR-H1, a CDR-H2 and a CDR-H3 contained within the V_{H} region amino acid sequence set forth in SEQ ID NO: 13 and the V_{L} region includes a CDR-L1, a CDR-L2 and a CDR-L3 contained within the V_{L} region amino acid sequence set forth in SEQ ID NO: 16. In some of any embodiments, the V_{H} region includes a CDR-H1, a CDR-H2 and a CDR-H3 contained within the V_{H} region amino acid sequence set forth in SEQ ID NO: 13 and the V_{L} region includes a CDR-L1, a CDR-L2 and a CDR-L3 contained within the V_{L} region amino acid sequence set forth in SEQ ID NO: 17.

In some of any embodiments, the V_{H} region includes a CDR-H1, a CDR-H2 and a CDR-H3 contained within the V_{H} region amino acid sequence set forth in SEQ ID NO: 13 and the V_{L} region includes a CDR-L1, a CDR-L2 and a CDR-L3 contained within the V_{L} region amino acid sequence set forth in SEQ ID NO: 18. In some of any embodiments, the V_{H} region includes a CDR-H1, a CDR-H2 and a CDR-H3 contained within the V_{H} region amino acid sequence set forth in SEQ ID NO: 13 and the V_{L} region includes a CDR-L1, a CDR-L2 and a CDR-L3 contained within the V_{L} region amino acid sequence set forth in SEQ ID NO: 19. In some of any embodiments, the V_{H} region includes a CDR-H1, a CDR-H2 and a CDR-H3 contained within the V_{H} region amino acid sequence set forth in SEQ ID NO: 14 and the V_{L} region includes a CDR-L1, a CDR-L2 and a CDR-L3 contained within the V_{L} region amino acid sequence set forth in SEQ ID NO: 16. In some of any embodiments, the V_{H} region includes a CDR-H1, a CDR-H2 and a CDR-H3 contained within the V_{H} region amino acid sequence set forth in SEQ ID NO: 14 and the V_{L} region includes a CDR-L1, a CDR-L2 and a CDR-L3 contained within the V_{L} region amino acid sequence set forth in SEQ ID NO: 17. In some of any embodiments, the V_{H} region includes a CDR-H1, a CDR-H2 and a CDR-H3 contained within the V_{H} region amino acid sequence set forth in SEQ ID NO: 14 and the V_{L} region includes a CDR-L1, a CDR-L2 and a CDR-L3 contained within the V_{L} region amino acid sequence set forth in SEQ ID NO: 18. In some of any embodiments, the V_{H} region includes a CDR-H1, a CDR-H2 and a CDR-H3 contained within the V_{H} region amino acid sequence set forth in SEQ ID NO: 14 and the V_{L} region includes a CDR-L1, a CDR-L2 and a CDR-L3 contained within the V_{L} region amino acid sequence set forth in SEQ ID NO: 19.

In some of any embodiments, the V_{H} region includes a CDR-H1, a CDR-H2 and a CDR-H3 contained within the V_{H} region amino acid sequence set forth in SEQ ID NO: 15 and the V_{L} region includes a CDR-L1, a CDR-L2 and a CDR-L3 contained within the V_{L} region amino acid sequence set forth in SEQ ID NO: 16. In some of any embodiments, the V_{H} region includes a CDR-H1, a CDR-H2 and a CDR-H3 contained within the V_{H} region amino acid sequence set forth in SEQ ID NO: 15 and the V_{L} region includes a CDR-L1, a CDR-L2 and a CDR-L3 contained within the V_{L} region amino acid sequence set forth in SEQ ID NO: 17. In some of any embodiments, the V_{H} region includes a CDR-H1, a CDR-H2 and a CDR-H3 contained within the V_{H} region amino acid sequence set forth in SEQ ID NO: 15 and the V_{L} region includes a CDR-L1, a CDR-L2 and a CDR-L3 contained within the V_{L} region amino acid sequence set forth in SEQ ID NO: 18. In some of any embodiments, the V_{H} region includes a CDR-H1, a CDR-H2 and a CDR-H3 contained within the V_{H} region amino acid sequence set forth in SEQ ID NO: 15 and the V_{L} region includes a CDR-L1, a CDR-L2 and a CDR-L3 contained within the V_{L} region amino acid sequence set forth in SEQ ID NO: 19.

In some of any embodiments, the V_{H} region includes a heavy chain complementarity determining region 1 (CDR-H1) including the amino acid sequence set forth in SEQ ID NO: 20, a heavy chain complementarity determining region 2 (CDR-H2) including the amino acid sequence set forth in SEQ ID NO: 21, and a heavy chain complementarity determining region 3 (CDR-H3) including the amino acid sequence set forth in SEQ ID NO: 22, and the V_{L} region includes a light chain complementarity determining region 1 (CDR-L1) including the amino acid sequence set forth in SEQ ID NO: 23, a light chain complementarity determining region 2 (CDR-L2) including the amino acid sequence set forth in SEQ ID NO: 24, and a light chain complementarity determining region 3 (CDR-L3) including the amino acid sequence set forth in SEQ ID NO: 25.

In some of any embodiments, the V_{H} region includes a CDR-H1 including the amino acid sequence set forth in SEQ ID NO: 26, a CDR-H2 including the amino acid sequence set forth in SEQ ID NO: 27, and a CDR-H3 including the amino acid sequence set forth in SEQ ID NO: 28, and the V_{L} region includes a CDR-L1 including the amino acid sequence set forth in SEQ ID NO: 29, a CDR-L2 including the amino acid sequence set forth in SEQ ID NO: 24, and a CDR-L3 including the amino acid sequence set forth in SEQ ID NO: 30.

In some of any embodiments, the V_{H} region includes a CDR-H1 including the amino acid sequence set forth in SEQ ID NO: 31, a CDR-H2 including the amino acid sequence set forth in SEQ ID NO: 32, and a CDR-H3 including the amino acid sequence set forth in SEQ ID NO: 33, and the V_{L} region includes a CDR-L1 including the amino acid sequence set forth in SEQ ID NO: 34, a CDR-L2 including the amino acid sequence set forth in SEQ ID NO: 35, and a CDR-L3 including the amino acid sequence set forth in SEQ ID NO: 36. In some of any embodiments, the V_{H} region includes a CDR-H1 including the amino acid sequence set forth in SEQ ID NO: 37, a CDR-H2 including the amino acid sequence set forth in SEQ ID NO: 38, and a CDR-H3 including the amino acid sequence set forth in SEQ ID NO: 39, and the V_{L} region includes a CDR-L1 including the amino acid sequence set forth in SEQ ID NO: 40, a CDR-L2 including the amino acid sequence set forth in SEQ ID NO: 41, and a CDR-L3 including the amino acid sequence set forth in SEQ ID NO: 42.

In some of any embodiments, the conjugate includes an antibody or antigen-binding fragment, which includes a heavy chain variable (V_{H}) region and a light chain variable (V_{L}) region. In some of any embodiments, the V_{H} region includes the amino acid sequence set forth in SEQ ID NO: 1 and the V_{L} region includes the amino acid sequence set forth in SEQ ID NO: 2. In some of any embodiments, the V_{H} region includes the amino acid sequence set forth in SEQ ID NO: 3 and the V_{L} region includes the amino acid sequence set forth in SEQ ID NO: 4. In some of any embodiments, the V_{H} region includes the amino acid sequence set forth in SEQ ID NO: 5 and the V_{L} region includes the amino acid sequence set forth in SEQ ID NO: 6. In some of any embodiments, the V_{H} region includes the amino acid sequence set forth in SEQ ID NO: 7 and the V_{L} region includes the amino acid sequence set forth in SEQ ID NO: 8. In some of any embodiments, the V_{H} region includes the amino acid sequence set forth in SEQ ID NO: 9 and the V_{L} region includes the amino acid sequence set forth in SEQ ID NO: 11. In some of any embodiments, the V_{H} region includes the amino acid sequence set forth in SEQ ID NO: 9 and the V_{L} region includes the amino acid sequence set forth in SEQ ID NO: 12. In some of any embodiments, the V_{H} region includes the amino acid sequence set forth in SEQ ID NO: 10 and the V_{L} region includes the amino acid sequence set forth in SEQ ID NO: 11. In some of any embodiments, the V_{H} region includes the amino acid sequence set forth in SEQ ID NO: 10 and the V_{L} region includes the amino acid sequence set forth in SEQ ID NO: 12. In some of any embodiments, the V_{H} region includes the amino acid sequence set forth in SEQ ID NO: 13 and the V_{L} region includes the amino acid sequence set forth in SEQ ID NO: 16. In some of any embodiments, the V_{H} region includes the amino acid sequence set forth in SEQ ID NO: 13 and the V_{L} region includes the amino acid sequence set forth in SEQ ID NO: 17. In some of any embodiments, the V_{H} region includes the amino acid sequence set forth in SEQ ID NO: 13 and the V_{L} region includes the amino acid sequence set forth in SEQ ID NO: 18. In some of any embodiments, the V_{H} region includes the amino acid sequence set forth in SEQ ID NO: 13 and the V_{L} region includes the amino acid sequence set forth in SEQ ID NO: 19. In some of any embodiments, the V_{H} region includes the amino acid sequence set forth in SEQ ID NO: 14 and the V_{L} region includes the amino acid sequence set forth in SEQ ID NO: 16. In some of any embodiments, the V_{H} region includes the amino acid sequence set forth in SEQ ID NO: 14 and the V_{L} region includes the amino acid sequence set forth in SEQ ID NO: 17. In some of any embodiments, the V_{H} region includes the amino acid sequence set forth in SEQ ID NO: 14 and the V_{L} region includes the amino acid sequence set forth in SEQ ID NO: 18. In some of any embodiments, the V_{H} region includes the amino acid sequence set forth in SEQ ID NO: 14 and the V_{L} region includes the amino acid sequence set forth in SEQ ID NO: 19. In some of any embodiments, the V_{H} region includes the amino acid sequence set forth in SEQ ID NO: 15 and the V_{L} region includes the amino acid sequence set forth in SEQ ID NO: 16. In some of any embodiments, the V_{H} region includes the amino acid sequence set forth in SEQ ID NO: 15 and the V_{L} region includes the amino acid sequence set forth in SEQ ID NO: 17. In some of any embodiments, the V_{H} region includes the amino acid sequence set forth in SEQ ID NO: 15 and the V_{L} region includes the amino acid sequence set forth in SEQ ID NO: 18. In some of any embodiments, the V_{H} region includes the amino acid sequence set forth in SEQ ID NO: 15 and the V_{L} region includes the amino acid sequence set forth in SEQ ID NO: 19.

In some of any embodiments, the antibody or antigen-binding fragment includes MA251, 7G7B6 or an antigen-binding portion thereof. In some of any embodiments, the antibody or antigen-binding fragment is a human, chimeric, or humanized antibody or antigen-binding fragment. In some of any embodiments, the conjugate includes an IgG1 Fc region or an IgG1 isotype, an IgG2 Fc region or an IgG2 isotype, IgG3 Fc region or an IgG3 isotype, or an IgG4 Fc region or an IgG4 isotype.

In some of any embodiments, antibody or antibody-binding fragment includes an IgG1 Fc region or an IgG1 isotype. In some of any embodiments, the IgG1 Fc region does not exhibit enhanced antibody-dependent cellular cytotoxicity (ADCC) effector function.

In some of any embodiments, the antibody or antibody-binding fragment includes an IgG2 Fc region or an IgG2 isotype. In some of any embodiments, the IgG2 Fc region comprises a substitution that reduces or abrogates ADCC effector function. In some of any embodiments, the substitution is a substitution of glutamine for the asparagine in the Fc region at the position corresponding to 297 according to EU numbering (N297Q).

In some of any embodiments, the conjugate exhibits one or more Fc-mediated effector function(s). In some of any embodiments, the conjugate lacks Fc-mediated effector function(s), exhibits substantially reduced Fc-mediated effector function(s) or does not exhibit substantial Fc-mediated effector function(s). In some of any embodiments, the activated conjugate is capable of cell killing in the absence of substantial Fc-mediated effector function(s). In some of any embodiments, the Fc-mediated effector function is selected from one or more of an antibody-dependent cellular cytotoxicity (ADCC), antibody-dependent cellular phagocytosis (ADCP) or complement-dependent cytotoxicity (CDC). In some of any embodiments, the antibody or antigen binding fragment includes an Fc region of a human immunoglobulin and/or human antibody framework regions.

Provided herein are methods of treating a tumor or a lesion in a subject. In some of any embodiments, the provided methods involve administering to the subject any of the conjugates provided herein; and illuminating a target site within the subject with a wavelength of between at or about 600 nm and at or about 850 nm, and at a dose of from at or about 25 J/cm² to at or about 400 J/cm² or from at or about 2 J/cm fiber length to at or about 500 J/cm fiber length, thereby activating the conjugate.

Provided herein are methods of treating a tumor or a lesion in a subject that involve administering to the subject a conjugate, wherein the conjugate includes an antibody or antigen-binding fragment that specifically binds a CD25 without substantially blocking or interfering with IL-2 signaling, and a Si-phthalocyanine dye; and illuminating a target site within the subject with a wavelength of between at or about 600 nm and at or about 850 nm, and at a dose of from at or about 25 J/cm² to at or about 400 J/cm² or from at or about 2 J/cm fiber length to at or about 500 J/cm fiber length, thereby activating the conjugate.

In some of any embodiments, the Si-phthalocyanine dye is IR700 and the illuminating step is performed at a wavelength of 690 nm±20 nm.

In some of any embodiments, the Si-phthalocyanine dye comprises Formula (I)

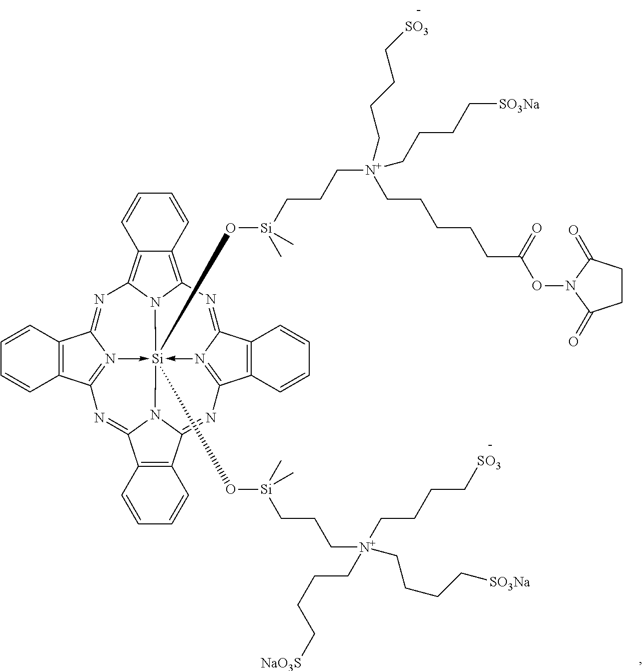

or is a salt, stereoisomer, or tautomer thereof, and the illuminating step is performed at a wavelength of 660 nm±50 nm.

In some of any embodiments, the growth, volume or dimensions of the tumor or the lesion is reduced or inhibited.

In some of any embodiments, the tumor or lesion to be treated, or the tumor microenvironment (TME) of the tumor or lesion to be treated, contains reduced levels of immune effector cells. In some of any embodiments, the tumor or lesion has a reduced response or is non-responsive to the unconjugated antibody.

In some of any embodiments, the immune effector cells are selected from one or more of macrophages, natural killer (NK) cells, neutrophils, and eosinophils.

In some of any embodiments, the target site is illuminated within about 24±4 hours after administering the conjugate. In some of any embodiments, target site is illuminated at an optical power of between at or about 50 mW/cm² and at or about 200 mW/cm², or between at or about 100 mW/cm fiber length and at or about 500 mW/cm fiber length. In some of any embodiments, the target site is illuminated for between at or about 120 seconds and at or about 600 seconds.

In some of any embodiments, the tumor or lesion is resistant or non-responsive to immune checkpoint inhibitor therapy.

In some of any embodiments, the conjugate includes an IgG1 Fc region or an IgG1 isotype, an IgG2 Fc region or an IgG2 isotype, IgG3 Fc region or an IgG3 isotype, or an IgG4 Fc region or an IgG4 isotype.

In some of any embodiments, the antibody or antibody-binding fragment includes an IgG1 Fc region or an IgG1 isotype. In some of any embodiments, the IgG1 Fc region is not enhanced for ADCC effector function. In some of any embodiments, the antibody or antibody-binding fragment includes an IgG2 Fc region or an IgG2 isotype. In some of any embodiments, the IgG2 Fc region comprises a substitution that reduces or abrogates ADCC effector function. In some of any embodiments, the substitution is a substitution of glutamine for the asparagine in the Fc region at the position corresponding to 297 according to EU numbering (N297Q).

In some of any embodiments, the conjugate exhibits one or more Fc-mediated effector function(s). In some of any embodiments, the conjugate lacks Fc-mediated effector function(s), exhibits substantially reduced Fc-mediated effector function(s) or does not exhibit substantial Fc-mediated effector function(s). In some of any embodiments, the activated conjugate is capable of cell killing in the absence of substantial Fc-mediated effector function(s). In some of any embodiments, the Fc-mediated effector function is selected from one or more of an antibody-dependent cellular cytotoxicity (ADCC), antibody-dependent cellular phagocytosis (ADCP) or complement-dependent cytotoxicity (CDC).

In some of any embodiments, the methods disclosed herein further involve administering an immune checkpoint inhibitor therapy subsequent to the administration of the conjugate. In some of any embodiments, the immune checkpoint inhibitor therapy is administered 1 day, 2 days, 3 days, 4 days, 5 days, 6 days, 7 days, 1 week, 2 weeks or 3 weeks following administration of the conjugate. In some of any embodiments, the immune checkpoint inhibitor therapy is administered 1 day, 2 days, 3 days, 4 days, 5 days, 6 days, 7 days, 1 week, 2 weeks or 3 weeks following the illumination. In some of any embodiments, the immune checkpoint inhibitor therapy is administered more than once following administration of the conjugate.

In some of any embodiments, the immune checkpoint inhibitor therapy includes a PD-1 inhibitor, a PD-L1 inhibitor or a CTLA-4 inhibitor. In some of any embodiments, the PD-1 inhibitor is an anti-PD-1 antibody selected from pembrolizumab (MK-3475, KEYTRUDA; lambrolizumab), nivolumab (OPDIVO), cemiplimab (LIBTAYO), toripalimab (JS001), HX008, SG001, GLS-010, dostarlimab (TSR-042), tislelizumab (BGB-A317), cetrelimab (JNJ-63723283), pidilizumab (CT-011), genolimzumab (APL-501, GB226), BCD-100, cemiplimab (REGN2810), F520, sintilimab (IBI308), CS1003, LZM009, camrelizumab (SHR-1210), SCT-I10A, MGA012, AK105, PF-06801591, AMP-224, AB122, AMG 404, BI 754091, HLX10, JTX-4014, AMP-514 (MEDI0680), Sym021, MGD019, MGD013, AK104, XmAb20717, R07121661, CX-188, spartalizumab, BCD-217, HX009, IB1308, PDR001, REGN2810, TSR-042 (ANB011), or an antigen-binding fragment thereof or any combination thereof.

In some of any embodiments, the population of regulatory T cells (Tregs) in the tumor or the lesion or in the tumor microenvironment is reduced as a result of the method.

In some of any embodiments, the reduction or inhibition includes one or more of less than 20% increase in tumor volume or tumor dimensions, or a reduction in tumor volume, tumor dimensions or tumor mass, or a reduction in number of tumor cells. In some of any embodiments, the growth, volume or dimensions of the tumor or the lesion is inhibited or reduced to a greater degree as compared to a method that employs a conjugate including an antibody or antigen-binding fragment that specifically binds a CD25 and substantially blocks or interferes with IL-2 signaling.

In some of any embodiments, the methods disclosed herein improve the overall survival of the subject. In some of any embodiments, the subject has a second tumor or secondary population of tumor cells, and the growth, volume or dimensions of the second tumor or secondary population of tumor cells is reduced or inhibited as a result of the method. In some of any embodiments, the second tumor or secondary population of tumor cells is not illuminated or has not been illuminated.

### BRIEF DESCRIPTION OF THE DRAWINGS

FIG. 1 shows the average tumor volume over time in mice with an implanted CT26 tumor that were administered an exemplary IL-2 non-blocking anti-CD25-mIgG1-IR700 conjugate (7D4-IR700; triangles) or an exemplary IL-2 blocking anti-CD25-IgG1-IR700 conjugate (PC61-IR700; squares), alone (solid lines) or followed by illumination at 690 nm at a dosage of 100 J/cm² (dashed lines). Control tumor-bearing mice were administered only saline (open circles).

FIGS. 2A-2E show the tumor growth of individual mice from FIG. 1.

FIG. 3 depicts the survival of mice from FIG. 1.

FIG. 4A shows the average tumor volume over time in mice with an implanted MCA205 murine fibrosarcoma tumor that were administered anti-PD1 antibody (open circles, dashed line), exemplary IL-2 non-blocking anti-CD25 antibody (7D4-mIgG2a; filled squares, solid line), or a combination of an exemplary IL-2 blocking anti-CD25 antibody and anti-PD-1 antibody (7D4-mIgG2a+a-PD1; open squares, dashed line). Control tumor-bearing mice were administered only saline (filled circles, solid line).

FIG. 4B shows the average tumor volume over time in mice with an implanted MCA205 murine fibrosarcoma tumor that were administered anti-PD1 antibody (open circles, dashed line), an exemplary IL-2 non-blocking anti-CD25-mIgG1-IR700 conjugate, followed by illumination at 690 nm at a dosage of 200 J/cm² (7D4-mIgG2a-IR700+PIT; filled triangles, solid line) or a combination of an exemplary IL-2 non-blocking anti-CD25-mIgG1-IR700 conjugate, followed by illumination at 690 nm at a dosage of 200 J/cm² and anti-PD-1 antibody (7D4-mIgG2a-IR700 PIT+a-PD1; open triangles, dashed line). Control tumor-bearing mice were administered only saline (filled circles, solid line).

FIGS. 5A-5F show the tumor growth of individual mice from FIGS. 4A-4B.

FIG. 6 depicts the survival of mice from FIGS. 4A-4B.

FIG. 7A shows the anti-tumor effects of saline (open triangles, dashed line), an exemplary IL-2 blocking anti-CD25-IR700 conjugate alone (PC61-IR700; open circle, solid line) or followed by illumination at 690 nm at a dosage of 200 J/cm² (PC61-IR700 PIT; closed circle, solid line) in the illuminated tumor of mice with bilateral implanted MCA205 murine fibrosarcoma tumors.

FIG. 7B shows the anti-tumor effects of saline (open triangles, dashed line), an exemplary IL-2 non-blocking anti-CD25-IR700 conjugate alone (7D4-IR700; open square, solid line) or followed by illumination at 690 nm at a dosage of 200 J/cm² (closed square, solid line) in the illuminated tumor of mice with bilateral implanted MCA205 murine fibrosarcoma tumors.

FIG. 8A shows the anti-tumor effects of saline (open triangles, dashed line), an exemplary IL-2 blocking anti-CD25-IR700 conjugate alone (PC61-IR700; open circle, solid line) or followed by illumination at 690 nm at a dosage of 200 J/cm² (PC61-IR700 PIT; closed circle, solid line) in the non-illuminated, distal tumor of mice from FIG. 7A.

FIG. 8B shows the anti-tumor effects of saline (open triangles, dashed line), an exemplary IL-2 non-blocking anti-CD25-IR700 conjugate alone (7D4-IR700; open square, solid line) or followed by illumination at 690 nm at a dosage of 200 J/cm² (closed square, solid line) in the non-illuminated, distal tumor of mice from FIG. 7B.

FIG. 9 shows the antibody-dependent cellular cytotoxicity (ADCC) activity of an IL-2 non-blocking anti-CD25 antibody (7D4-mIgG2a) and the corresponding IR700 conjugate (7D4-mIgG2a-IR700).

FIG. 10 shows the average tumor volume over time in mice with an implanted CT26 tumor that were administered an exemplary IL-2 non-blocking antibody with a mouse IgG2a backbone (7D4-mIgG2a; closed squares), the corresponding ADCC/ADCP null IL-2 non-blocking antibody (7D4-mIgG2a-N297Q; open squares), and an exemplary IL-2 non-blocking anti-CD25-mIgG1-IR700 conjugate with a mouse IgG1 backbone alone (7D4-mIgG1-IR700; triangles) or followed by illumination at 690 nm at a dosage of 100 J/cm² (7D4-mIgG1-IR700+PIT, dashed line). Control tumor-bearing mice were administered only saline (open circles).

FIGS. 11A-11E show the tumor growth of individual mice from FIG. 10.

FIG. 12 depicts the survival of mice from FIG. 10.

FIG. 13 depicts the average tumor volume over time in mice with an implanted immunologically “cold” tumor that were administered an exemplary IL-2 non-blocking antibody with a mouse IgG2a backbone (7D4-mIgG2a), alone (open squares) or in combination with anti-PD-1 antibody (closed squares), the corresponding exemplary IL-2 non-blocking anti-CD25-mIgG1-IR700 conjugate followed by illumination at 690 nm at a dosage of 150 J/cm² alone (7D4-mIgG1-IR700 PIT; open triangles) or in combination with anti-PD-1 antibody (closed triangle). Control tumor-bearing mice were administered only saline (open circles) or anti-PD-1 (closed circles).

FIG. 14 depicts the survival of mice from FIG. 13.

### DETAILED DESCRIPTION

Provided herein are conjugates, compositions, combinations and methods for treating a cancer, such as cancers that include a first tumor or a primary tumor or multiple primary tumors, as well as a later-introduced tumor, such as a tumor comprising a secondary population of tumor cells, such as metastatic tumor cells, for example, metastatic cancers; and/or cancers that include a primary tumor or multiple primary tumors, as well as invasive or infiltrating tumor cells, for example, invasive cancers or infiltrating cancers. Also provided are conjugates, compositions, combinations and methods for enhancing systemic immunity in a subject, such as a subject having a cancer, such as an invasive cancer, an infiltrating cancer, or a metastatic cancer. Also provided are conjugates, compositions, combinations and methods for generating an enhanced response, for example, an enhanced response to a treatment or a therapy in a subject, e.g., a subject having a cancer or a tumor, such as an invasive cancer, an infiltrating cancer, or a metastatic cancer. In some contexts, the cancer exhibits diminished immunoresponsiveness (e.g., exhibiting low levels of and exhausted tumor-infiltrating lymphocytes (TILs), insufficient tumor antigen burden, and/or immunosuppressive microenvironment; also called “cold” tumors), and the provided conjugates, compositions, combinations, and/or methods effect treatment of the cancer, enhance the systemic immunity of the subject, and or generates and enhanced response to a treatment or therapy in the subject.

In any of the provided embodiments, the conjugates described herein can exhibit one or more mechanisms of anti-tumor effects. Thus, the conjugates provided herein, offer the ability to provide anti-tumor effects in a broader spectrum of tumor-types and tumor environments. The conjugates herein target CD25 antigen on the surface of cells and thus can specifically target CD25-expressing T cells for destruction according to the provided methods. In some aspects, the conjugates, although able to bind to CD25 antigen, do not substantially block or interfere with IL-2 signaling. In some aspects, the preservation of IL-2 signaling allows for stimulation of cytotoxic effector T cells and enhancement of activation-induced cell death (AICD), as well as promoting differentiation of T cells into effector T cells and into memory T cells. In some aspects, the conjugates also include a light-activated phthalocyanine dye. The conjugated dye allows for the activation of the targeted cell-killing when specific wavelengths of light are present or illuminated with specific wavelengths of light. In some aspects, different from an unconjugated anti-CD25 antibody, the targeted destruction of CD25-expressing T cells can be localized using the delivery of light. In addition, the conjugate is able to provide targeted cell killing in the absence of Fc-mediated effector function. In some aspects, the conjugates therefore provide cell killing in tumors and tumor microenvironments where antibody-dependent cell-mediated cytotoxicity (ADCC), antibody-dependent cellular phagocytosis (ADCP), and complement-dependent cytotoxicity (CDC) are reduced or impeded.

The provided conjugates, compositions, combinations, methods and uses can be used to treat cancers that include a first tumor or a primary tumor, metastatic tumor cells and/or invasive tumor cells. In some embodiments, a phthalocyanine dye conjugated with a targeting molecule that binds CD25 is used alone or in combination with an immune checkpoint inhibitor. The methods and uses described herein provide various advantages in treating cancers, e.g., cancers comprising a secondary population of tumor cells, metastatic cancers and/or invasive cancers, including without the need to locate and/or directly illuminate the metastatic tumor cells and/or invasive tumor cells. The disclosure also provides unexpected features in enhancing the systemic immunity in a subject, for example, against cancer recurrence.

The provided embodiments include or employ a targeting molecule that specifically binds CD25 but does not interfere with or block, such as does not substantially interfere with or substantially block, the binding of IL-2 to CD25, such as an IL-2 non-blocking anti-CD25 targeting antibody or an antigen-binding fragment thereof (e.g., also called an IL-2 non-blocking antibody). The provided embodiments include or employ IL-2 non-blocking anti-CD25 conjugates, such as conjugates comprising an IL-2 non-blocking anti-CD25 targeting antibody or an antigen-binding fragment thereof and a phthalocyanine dye with a silicon coordinating metal (Si-phthalocyanine dye). In any of the embodiments, the phthalocyanine dye is a salt or ionic form thereof. In any of the embodiments, the phthalocyanine dye is a salt, an ionic form, a stereoisomer, or a tautomer thereof.

CD25, also known as the alpha chain of the interleukin-2 receptor (IL-2Rα or IL-2Rα), is constitutively expressed at high-levels on regulatory T cells (Tregs) and activated T cells. In some cases, CD25 can also be expressed on cancer cells, such as on leukemic cells in some acute myeloid leukemias. IL-2 has essential roles in key functions during immune homeostasis, especially via direct effects on regulatory T cells and the optimizing and fine-tuning of effector lymphocyte responses (Arenas-Ramirez et al., (2015) Trends Immunol. 36(12):763-777). For example, in the thymus, where T cells mature, low levels of IL-2 signaling can promote the differentiation of certain immature T cells into Tregs, while high levels of IL-2 signaling can stimulate cytotoxic effector T cells which can promote anti-tumor responses. IL-2 can also enhance activation-induced cell death (AICD). IL-2 signaling can also promote the differentiation of T cells into effector T cells and into memory T cells when the initial T cell is also stimulated by an antigen (Liao et al., (2011) Curr Opin Immunol. 23(5):598-604. IL-2 expression and secretion is tightly regulated and functions as part of both transient positive and negative feedback loops in mounting and dampening immune responses. Through its role in the development of T cell immunologic memory, which depends upon the expansion of the number and function of antigen-selected T cell clones, it plays a key role in enduring cell-mediated immunity. Hence, in the provided embodiments, the IL-2 non-blocking anti-CD25 antibody or conjugate does not interfere with, such as does not substantially interfere with, these contributions of IL-2 to the immune response. In some embodiments, the provided methods and uses do not impede or block, such as does not substantially impede or block, IL-2 signaling systemically, external to the illuminated tumor or lesion.

In any of the provided embodiments, the conjugates described herein can exhibit one or more Fc-mediated effector functions. Fc-mediated effector functions include, but are not limited to, antibody-dependent cell-mediated cytotoxicity (ADCC), antibody-dependent cellular phagocytosis (ADCP), and complement-dependent cytotoxicity (CDC). In some embodiments, Fc-mediated effector functions (e.g., ADCC or ADCP) can be effected on CD25-expressing cells when bound to the IL-2 non-blocking anti-CD25 antibody conjugates provided herein. In some embodiments, the IL-2 non-blocking anti-CD25 antibody contains a functional Fc region to permit Fc-mediated effector functions (e.g., ADCC or ADCP) of the provided IL-2 non-blocking anti-CD25 conjugate.

In some embodiments, the IL-2 non-blocking anti-CD25 antibody contains a modified Fc region to enhance one or more Fc-mediated effector functions (e.g., ADCC and/or ADCP) of the provided IL-2 non-blocking anti-CD25 conjugate. Exemplary modifications to the Fc region include one or more amino acid substitution(s) within the Fc region and/or substitution of the entire Fc region with the Fc region of an antibody of a different isotype. In such embodiments, the provided methods effect targeted elimination of CD25-expressing cells via illumination and via Fc-mediated effector functions (e.g., ADCC or ADCP), such as ADCC-mediated elimination of CD25-expressing cells distal to the site of illumination, while also retaining IL-2 signaling and/or not substantially interfering with IL-2 signaling.

In some embodiments, the Fc region of the IL-2 non-blocking anti-CD25 antibody is non-functional or has reduced Fc-mediated effector function, such as a reduction in ADCC, ADCP, and/or CDC activity. In some of such embodiments, the Fc region of the targeting antibody is modified to reduce or eliminate Fc-mediated effector function (e.g., ADCC and/or ADCP). Such modifications include amino acid substitution, deletion, or truncation of some or all of the Fc region.

The provided embodiments, in some contexts, are based on the observation that treatment of a cancer with a phthalocyanine dye-targeting molecule conjugate, such as an IL-2 non-blocking anti-CD25 antibody-phthalocyanine dye conjugate (e.g., IL-2 non-blocking anti-CD25-IR700), followed by illumination of a first tumor or a primary tumor, results in not only treatment of the illuminated tumor, such as the illuminated first tumor or the illuminated primary tumor, but also results in effective treatment of a tumor that is distal to the illumination site (e.g., metastasized tumor), and effective treatment of a tumor that is introduced after the subject has a complete response following the treatment of the initial tumor, indicating a tumor-specific immune memory response. The provided embodiments are based on a further observation that a combination treatment with an IL-2 non-blocking anti-CD25 antibody-phthalocyanine dye conjugate (e.g., IL-2 non-blocking anti-CD25-IR700) and an immune checkpoint inhibitor, such as an anti-PD-1 antibody, results in striking synergistic effects in the treatment of both the illuminated first tumor or primary tumor and a distal tumor or a later-introduced tumor, such as a tumor comprising a secondary population of tumor cells, a metastatic tumor and/or an invasive tumor.

The provided embodiments, in some contexts, are based on the observation that treatment of a tumor with an IL-2 non-blocking anti-CD25 conjugate (e.g., IL-2 non-blocking anti-CD25-IR700) exhibits at least two modes of action to effect target cell killing. In some contexts, the tumor is a tumor with diminished immunoresponsiveness (e.g., exhibiting low levels of and exhausted tumor-infiltrating lymphocytes (TILs), insufficient tumor antigen burden, and/or immunosuppressive microenvironment), such as a “cold” tumor. In some contexts, a tumor with diminished immunoresponsiveness is not responsive or not substantially responsive to immune checkpoint inhibitor therapy, such as one or more of anti-PD-1, anti-PD-L1 or anti-CTLA-4 therapies. Accordingly, the provided conjugates, compositions, combinations, methods and uses are demonstrated to provide a substantially improved and effective treatment of a cancer, including cancers that include a first tumor or a primary tumor or multiple primary tumors as well as metastatic tumor cells, for example, metastatic cancers; and/or cancers that include a first tumor or a primary tumor or multiple primary tumors as well as invasive tumor cells, for example, invasive cancers. The provided conjugates, compositions, combinations, methods and uses can result in enhancement or improvement of the subject's immune response, e.g., systemic immune response against a cancer including immune memory response, that can be effective against tumors that may develop after the treatment.

The methods and uses provided herein include treating a subject that has one or more first tumors, e.g., primary tumors, and optionally, secondary population of cells, such as metastatic tumor cells and/or invasive tumor cells with a conjugate comprising a phthalocyanine dye linked to a targeting molecule, such as a targeting molecule that binds to CD25, and after administration of the conjugate, illuminating the one or more first tumors or primary tumors with a light wavelength suitable for use with the phthalocyanine dye. Some embodiments of the method include administering an immune checkpoint inhibitor prior to, concurrent with or subsequent to administration of the conjugate.

### I. METHODS OF TREATMENT WITH AND USES OF IL-2 NON-BLOCKING ANTI-CD25 CONJUGATES

In some embodiments, the provided methods and uses involve administering an anti-CD25 conjugate, such as a conjugate comprising an anti-CD25 antibody that does not substantially block access or binding of IL-2 to CD25 when the antibody is bound or does not substantially interfere with or impair IL-2 mediated signaling through CD25 (i.e., an IL-2 non-blocking anti-CD25 antibody) and a phthalocyanine dye, and illumination of a target area, with a wavelength of light suitable for use with the phthalocyanine dye, such that the light excites the dye and results in killing of a cell that expresses CD25 on its surface.

In some aspects, provided are IL-2 non-blocking anti-CD25 conjugates. In such embodiments, IL-2 is capable of binding CD25 while the anti-CD25 conjugate is also bound to CD25, and IL-2 mediated signaling through binding to CD25 is not impaired in cells within or distal to the illuminated target area. IL-2 mediated activities include cytotoxic T lymphocyte expansion and other immunoregulatory activities, such as those described in Ross and Cantrell (2018) Annu Rev Immunol. 36: 411-433. Such methods result in enhancing, activating, inducing, provoking, augmenting, or supporting immune function, such as local and/or systemic immunity, reducing or eliminating a lesion (e.g., tumor), reducing or inhibiting tumor growth, reducing, inhibiting, or eliminating tumor cell metastasis, or any combination thereof. In some embodiments, the provided methods and uses effect therapy or treatment of a cancer, such as a cancer that includes a first tumor or multiple tumors (e.g., one or more primary tumors) as well as a secondary population of cancer cells, such as metastatic tumor cells (e.g., metastatic cancers), invasive tumor cells, (e.g., invasive cancers), or infiltrating tumor cells (e.g., infiltrating cancers). In some embodiments, the secondary population of cancer cells is related, such as directly or indirectly, to the first tumor. In some embodiments, the secondary population of cells is not directly derived from the first tumor.

The provided methods and uses include therapeutic methods and uses, for example, involving administration of the conjugates to a subject having a cancer followed by illumination (or irradiation) of a tumor associated with the cancer (such as a first tumor) or the microenvironment of a tumor, using a particular light wavelength and dose. In some aspects, the illumination (or irradiation) results in illumination-dependent lysis and death of cells expressing the CD25 target molecule, resulting in a therapeutic effect or treatment of the cancer (in some cases called photoimmunotherapy (PIT)). In some aspects, the methods also involve administration of an immune modulating agent, such as an immune checkpoint inhibitor (e.g., anti-PD-1 antibody), in combination with the phthalocyanine dye-targeting molecule conjugate. In some aspects, a combination of a phthalocyanine dye-targeting molecule conjugate and the immune checkpoint inhibitor (e.g., anti-PD-1 antibody) are employed in the provided methods and uses, such as in the provided methods and uses for treatment of a cancer, for example, as a combination therapy or treatment.

Uses include uses of the compositions described herein and combinations in such methods and treatments and uses of such compositions and combinations in the preparation of a medicament in order to carry out such therapeutic methods. In some aspects, such therapeutic methods include a combination therapy. In some embodiments, the methods and uses thereby treat the cancer, such as cancers that include tumors and cancers that include a first tumor, that is primary or non-primary tumor, and one or more secondary population(s) of tumor cells (e.g., metastatic tumor cells and/or invasive tumor cells), such as metastatic and/or invasive cancers, in a subject. In some embodiments the secondary tumor cells are related to the first tumor. In some embodiments of the methods and uses more than one tumor is treated. In some aspects, also provided are methods and uses of such conjugates, compositions and combinations in enhancing, boosting, augmenting, strengthening, increasing, boosting or supporting the immune function, such as systemic immunity, in the subject.

The methods include administering to a subject having a first tumor, a conjugate comprising a phthalocyanine dye linked to targeting molecule, wherein the targeting molecule binds to CD25 and after administration of the conjugate illuminating at least a first tumor with a wavelength of light suitable for the selected phthalocyanine dye. In some embodiments, the methods include the administration of an immune checkpoint inhibitor, such as an anti-PD-1 antibody, prior to, concurrent with or subsequent to the administration of the conjugate. In some embodiments the methods further administering an additional therapeutic agent or anti-cancer treatment.

In some embodiments, the methods involve illumination of a tumor associated with a cancer or the microenvironment of a tumor (tumor microenvironment; TME), or cells that are present in the TME, with light. In some aspects, the tumor or the TME is illuminated with a wavelength of light suitable for therapy or treatment. In some embodiments, a light wavelength suitable for use with the phthalocyanine dye includes light with a wavelength that achieves activation of the dye-conjugate by illumination with absorbing light, such that the light excites the photosensitizer and results in cell killing, thereby reducing or eliminating a lesion (e.g., tumor), reducing or inhibiting tumor growth, reducing, inhibiting or eliminating secondary populations of tumor cells, such as a tumor cell metastasis, reducing, inhibiting or eliminating invasive and/or metastatic tumor cells or any combination thereof.

In some embodiments, the illumination is at a wavelength between about 500 nm and 900 nm, between about 600 nm and 850 nm, between about 650 nm and 800 nm or between about 660 nm and 740 nm. In some embodiments, illumination is at a wavelength of 690±50 nm or at a wavelength of or about 690±20 nm.

The illumination can be at a dose of at least 1 J/cm² or 1 J/cm of fiber length. In some embodiments, the lesion is illuminated at a dose of from at or about 2 J/cm² to at or about 400 J/cm² or from at or about 2 J/cm fiber length to at or about 500 J/cm fiber length. In some embodiments, the illumination is at a dose of at least at or about 2 J/cm², 5 J/cm², 10 J/cm², 25 J/cm², 50 J/cm², 75 J/cm², 100 J/cm², 150 J/cm², 200 J/cm², 300 J/cm², 400 J/cm², or 500 J/cm²; or the lesion is illuminated at a dose of at least at or about 2 J/cm fiber length, 5 J/cm fiber length, 10 J/cm fiber length, 25 J/cm fiber length, 50 J/cm fiber length, 75 J/cm fiber length, 100 J/cm fiber length, 150 J/cm fiber length, 200 J/cm fiber length, 250 J/cm fiber length, 300 J/cm fiber length, 400 J/cm fiber length or 500 J/cm fiber length.

In some embodiments, the illumination is at a dose of from about 25 J/cm² to about 400 J/cm² or from about 2 J/cm fiber length to about 500 J/cm fiber length. In some embodiments, the illumination is at a dose from 5 J/cm² to 200 J/cm² or from 20 J/cm fiber length to 500 J/cm fiber length. In some embodiments, the illumination is at a dose of about 50 J/cm² or 100 J/cm fiber length.

In some embodiments of the methods and uses provided herein, the optical power (or optical fluence) of the light is from at or about 20 J/cm fiber length to at or about 500 J/cm fiber length. In some embodiments, the optical power (or optical fluence) of the interstitial light dose is at or about 100 mW/cm fiber length to at or about 500 mW/cm fiber length. In some embodiments, the light is administered for at or about 120 seconds to at or about 600 seconds. In some embodiments, the illumination is administered at a dose of at or about 100 J/cm fiber length with an optical fluence of at or about 400 mW/cm for at or about 250 seconds.

In some embodiments of the methods and uses provided herein, the optical power (or optical fluence) of the light is from at or about 25 J/cm² to at or about 400 J/cm². In some embodiments, the optical power (or optical fluence) of the light dose is at or about 50 mW/cm² to at or about 200 mW/cm². In some embodiments, the light is administered for at or about 120 seconds to at or about 600 seconds. In some embodiments, the illumination is administered at a dose of at or about 50 J/cm² at an optical power of 150 mW/cm² for at or about 333 seconds.

In some embodiments of the methods and uses provided herein, the illumination is administered after administration of the phthalocyanine dye-targeting molecule conjugate (e.g., IL-2 non-blocking anti-CD25 antibody-IR700 conjugate). In some embodiments, the illumination or illumination is carried out or effected between or between about 30 minutes and 96 hours after administering the phthalocyanine dye-targeting molecule conjugate, such as between 30 minutes and 48 hours, 30 minutes and 24 hours or 12 hours and 48 hours, such as generally at least 30 minutes, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours or more after administering the conjugate. In some embodiments, the illumination is performed within about 24 hours after administering the conjugate or within 24 hours±4 hours after administering the conjugate, or within about 20, 21, 22, 23, 24, 24, 26, 27, or 28 hours after administering the conjugate.

The methods described herein include illuminating a first tumor in a subject, or the tumor microenvironment (TME) of a first tumor, such as a primary tumor. In some embodiments, the methods and uses provided herein include treating a subject that has one or more tumors. The subject may have one, two, three, or more than three tumors. Such tumors can be in one or more tissues or organs, such as in one tissue or organ, in two different tissues or organs, in three different tissues or organs, or in more than three different tissues or organs.

In some aspects, primary tumor can refer to the first or original tumor in a subject and can also refer to the one or more tumors selected for illumination with the methods and uses provided herein. In some embodiments, a first tumor or additional tumors may be a solid tumor or solid tumors, may be lymphomas, or may be leukemias. The tumor can be tumor of the lung, stomach, liver, pancreas, breast, esophageal, head and neck, brain, peripheral nerve, skin, small intestine, colon, rectum, anus, ovary, uterus, bladder, prostate, adipose tissue, skeletal muscle, smooth muscle, blood vessel, bone, bone marrow, eye, tongue, lymph node, spleen, kidney, cervix, male genital, female genital, testis, or unknown primary origin.

In some embodiments of the methods, the growth of the first tumor or primary tumors is inhibited, the volume of the primary tumor or primary tumors is reduced or both tumor growth and volume are reduced. In some embodiments of the methods, the growth of the first tumor or primary tumors is inhibited, the volume of the primary tumor or primary tumors is reduced or both tumor growth and volume are reduced as compared to a monotherapy such as administration of only the conjugate, only the conjugate followed by illumination, or only the anti-PD-1 antibody.

In some embodiments, the methods and uses provided herein include treating a subject that has one or more tumors and also a secondary population of tumor cells, such as invasive tumor cells. In some of such embodiments, the secondary population contains when cells originating from a tumor, such as a primary tumor, have invaded into surrounding tissues. The methods include administering to a subject having a first tumor(s) and invasive tumor cells, a conjugate comprising a phthalocyanine dye linked to targeting molecule, wherein the targeting molecule binds to CD25 and after administration of the conjugate, illuminating the first tumor with a wavelength suitable for the selected phthalocyanine dye. In some of such embodiments, the secondary population of tumor cells is not directly illuminated. In some embodiments, the methods include the administration of an immune checkpoint inhibitor prior to, concurrent with or subsequent to the administration of the conjugate. In some embodiments, the methods include the administration of an anti-PD-1 antibody prior to, concurrent with, or subsequent to administration of an IL-2 non-blocking anti-CD25 antibody-IR700 conjugate, whereby the conjugate administration is followed by illumination of the first tumor.

In some aspects, invasive tumor cells refer to cells originated from a primary tumor and have invaded into surrounding tissues of the same organ or neighboring organs of the primary tumor within the body of a subject having the first tumor. In some embodiments, the first tumor is a primary tumor, and the invasive tumor cells are directly or indirectly derived from the first tumor. In some embodiments, the invasive tumor cells are not directly derived from the first tumor.

The methods and uses provided herein include illumination of the first tumor and/or additional tumors, and some or all of the invasive tumor cells are not illuminated. In some embodiments, the growth of invasive tumor cells is inhibited, reduced or eliminated, the volume, dimension, or mass of one or more invasive tumors is reduced or any combination thereof. In some embodiments, the growth of the first tumor also is inhibited, reduced or eliminated, the volume, dimension, or mass of the first tumor or additional tumors also is reduced along with the effect(s) on the one or more invasive tumor cells.

In some embodiments, invasive tumor cells are contained in a solid tumor. In some embodiments, invasive tumor cells are contained in body fluids, including but not limited to peritoneal fluid, pleural fluid, and cerebrospinal fluid. In some embodiments, invasive tumor cells are contained in the effusion of a body cavity or body cavities, including but not limited to peritoneal effusion (ascites), pleural effusion, and pericardial effusion.

In some embodiments, the methods and uses provided herein include treating a subject that has a first tumor and also a secondary population of tumor cells (for example, a secondary population of related tumor cells), such as invasive and/or metastatic tumor cells. The methods include administering to a subject having a first tumor and a secondary population of tumor cells (for example, a secondary population of related tumor cells), such as invasive and/or metastatic tumor cells, a conjugate comprising a phthalocyanine dye linked to targeting molecule, wherein the targeting molecule binds to CD25 and after administration of the conjugate, illuminating the first tumor with a wavelength suitable for the selected phthalocyanine dye. In some of such embodiments, the secondary population of tumor cells is not directly illuminated. In some embodiments, the methods include the administration of an immune checkpoint inhibitor, such as an anti-PD-1 antibody, prior to, concurrent with or subsequent to the administration of the conjugate. In such methods, the growth (volume, dimension, or mass) of the first tumor and/or the secondary population of tumor cells, such as metastatic tumor cells, is inhibited, reduced or eliminated, the volume, dimension, or mass of one or more of the first tumor and/or secondary population of cells is reduced, or any combination thereof. In some embodiments, the inhibition of the first tumor and/or secondary population is effected to a greater degree than the inhibition achieved by administration of only the conjugate, only the conjugate followed by illumination, or only the anti-PD-1 antibody. In some embodiments, inhibition is achieved if the tumor exhibits less than 20% increase in tumor volume, tumor dimension(s), or tumor mass; no change in tumor volume, dimension, or mass (i.e., halted tumor growth or progression); or the tumor is reduced in volume, dimensions, or mass; or there is a reduction in the number of tumor cells. In some aspects, the reduction in tumor volume, tumor dimensions or tumor mass, or the number of tumor cells comprises at or about a 30% reduction or more.

In any of the methods and uses herein, the first tumor can be a primary tumor or a secondary tumor. In some embodiments, the first tumor and the secondary population of tumor cells are related. In some embodiments, the secondary population of cells is directly or indirectly derived from the first tumor. In some embodiments, the secondary population is not derived from the first tumor. In some embodiments, the first tumor is a primary tumor and the secondary population of cells is related to the primary tumor; for example, the secondary population of cells is derived directly or indirectly from the primary tumor. In some embodiments, the first tumor is a primary tumor and the secondary population of tumor cells is a second primary tumor. In some embodiments, the first tumor is a secondary tumor and the secondary population of cells is related to the secondary tumor. In some aspects, a secondary population of tumor includes cells that originated from a primary tumor and invade local or distal healthy tissue (i.e., invasive tumor cells) or spread to a distal tissue or organ, or distal tissues or organs within the body of a subject having the primary tumor (i.e., metastatic tumor cells), for example, a tissue or an organ located distantly or far away from the primary tumor. In some aspects a secondary population of tumor cells is both invasive and metastatic. In some aspects a secondary population of tumor cells is infiltrating. In some aspects, a secondary population of tumor cells is metastatic and is directly or indirectly related to, such as derived from, the first tumor. In other aspects, a secondary population of tumor cells is metastatic and not directly related to the first tumor. Metastatic tumor cells can be located in one or more locations in the lung, stomach, liver, pancreas, breast, esophageal, head and neck, brain, peripheral nerve, skin, small intestine, colon, rectum, anus, ovary, uterus, bladder, prostate, adipose tissue, skeletal muscle, smooth muscle, blood vessel, bone, bone marrow, eye, tongue, lymph node, spleen, kidney, cervix, male genital, female genital, testis, blood, bone marrow, cerebrospinal fluid, or any other tissues or organs. In some embodiments, metastatic tumor cells are contained in a solid tumor. In some embodiments, metastatic tumor cells are circulating tumor cells, are a liquid tumor, or are not associated with a tumor mass.

In some embodiments of the methods and uses provided herein, secondary tumor cells are metastatic tumor cells that are distal to the first tumor and some or all of the metastatic tumor cells are not illuminated, e.g., not directly illuminated. In some embodiments of the methods and uses, only the first tumor is illuminated after administration of the conjugate and invasive or metastatic tumor cells are not directly illuminated. In some embodiments, more than one tumor, including a first tumor, is illuminated but at least one site of tumor cells, such as a site containing metastatic tumor cells, is not illuminated.

### II. METHODS FOR ENHANCING SYSTEMIC IMMUNITY AND/OR RESPONSE

Also provided herein are methods and uses of compositions and combinations in enhancing, boosting, augmenting, or supporting the immune function, such as systemic immunity, in a subject, e.g., in a subject having a cancer or a tumor. In some embodiments, the method and uses herein includes enhancing systemic immunity in a subject having a cancer, a tumor or a cancerous lesion. In some aspects, “systemic immunity” refers to the ability of a subject's immune system to respond to an immunologic challenge, including those associated with a cancer or a tumor, in a systemic manner. In some aspects, systemic immunity can include systemic response of the subject's adaptive immune system and/or innate immune system. In some aspects, systemic immunity includes an immune response across different tissues, including the blood stream, lymph node, bone marrow, spleen and/or the tumor microenvironment, and in some cases, includes a coordinated response among the tissues and organs and various cells and factors of the tissues and organs. Also provided herein are methods and uses of compositions and combinations in enhancing, boosting or augmenting a response to a treatment or a therapy in a subject, such as in a subject having a cancer or a tumor.

In some aspects, the provide methods and uses include administering to the subject a conjugate comprising a phthalocyanine dye linked to targeting molecule, wherein the targeting molecule binds to CD25 without blocking, such as without substantially blocking, binding or signaling of IL-2, administering an immune checkpoint inhibitor, and after administration of the conjugate, illuminating the tumor or a cancerous lesion, or the tumor microenvironment. The conditions for illumination regarding wavelength, dosage of illumination and timing of illumination are such as those described herein. The immune checkpoint inhibitor can be administered prior to, concurrent with or subsequent to the administration of the conjugate, such as described herein. In some aspects, the methods and uses provided herein result in an enhancement of the systemic immunity in the subject, which can in turn result in enhanced or synergistic response to the therapy or treatment for cancer. In some embodiments, the methods and uses provided herein results in an enhanced response, such as a synergistic response, to the treatment or therapy for the cancer or the tumor, as compared with the administration of only the conjugate, only the conjugate followed by illumination or only the anti-PD-1 antibody. In some aspects, the enhanced response comprises an enhancement of systemic immunity of the subject as compared to the systemic immunity of the subject prior to the administration of the conjugate followed by illumination and the anti-PD-1 antibody. In some aspects, the enhanced response comprises an enhanced response, such as n additional, additive, or synergistic response, and/or a more complete response, more durable response or longer lasting response, to the treatment as compared with administration of only the conjugate, only the conjugate followed by illumination, or only the anti-PD-1 antibody.

In some embodiments of the methods and uses provided herein, the systemic immunity against recurring tumors is increased or augmented. In some aspects, the level, strength or extent of systemic immunity can be measured based on the number of intratumoral CD8⁺ T lymphocytes, the ratio of CD8⁺ T lymphocytes to regulatory T cells (Tregs), intratumoral T lymphocyte exhaustion (e.g., the percentage of CD3⁺CD8⁺ cells that express PD-1 and/or CTLA4 markers), the number or percentage of intratumoral activated CD8⁺ T lymphocytes (e.g., Ki67⁺ or CD69⁺ CD8 cells as a percentage of CD45⁺ cells), the expansion of cytotoxic intratumoral T lymphocytes (e.g., the percentage of CD3⁺CD8⁺ cells that do not express PD-1 and/or CTLA4 markers), based on the splenocyte cytotoxicity against tumor cells, or any or all of combination thereof. In some aspects, intratumoral CD8⁺ T lymphocytes, exhausted intratumoral T lymphocytes, activated CD8⁺ T lymphocytes, or expanded cytotoxic intratumoral T lymphocytes are measured as a percentage of leukocytes (CD45⁺ cells) and/or total CD8⁺ T cells (e.g., CD3+CD8+CD45⁺ cells). Determination of such numbers or percentages can be achieved using several well-known methods, including those described herein. For example, such numbers or percentages can be determined by generating single cell suspensions, such as by mechanical dissociation of tumor and/or tissue biopsies, or collection of blood samples containing circulating immune cells, followed by staining and flow cytometric analysis or mass cytometry. Other methods can include multiplexed immunofluorescence imaging of tissue and/or tumor biopsies.

In some of such embodiments, the strength or extent of immunity is compared to the strength or extent of immunity in the same subject prior to treatment. In some of such embodiments, the strength or extent of immunity is compared to a population of subjects. In some of such embodiments, the strength or extent of immunity is compared to a threshold value. In some embodiments, the strength or extent of immunity following a combination therapy, such as IL-2 non-blocking anti-CD25 PIT (e.g., including light illumination for activation of the conjugate) in combination with administration of a checkpoint inhibitor (e.g., anti-PD-1 antibody), is compared to the strength or extent of immunity following treatment with a monotherapy, such as administration of a single agent, such as an immune checkpoint inhibitor (e.g., anti-PD-1 antibody) or IL-2 blocking or IL-2 non-blocking anti-CD25 conjugate, or IL-2 blocking or IL-2 non-blocking anti-CD25 PIT alone.

In some embodiments, treatment in accordance with the methods and uses provided herein, leads to cell death of or a reduction in number of regulatory T cells (Tregs), such as intratumoral CD4⁺ FoxP3⁺ Tregs. Hence, in some embodiments, the level, strength, or extent of systemic immunity can be measured based on the number or percentage of intratumoral or circulating regulatory T cells (Tregs). In some aspects, binding of the IL-2 non-blocking anti-CD25 conjugate to the surface of CD25-expressing cells, such as certain Tregs, and illumination to effect illumination-dependent lysis and death of cells expressing CD25, results in a reduction of the number of cells expressing CD25. In some aspects, such a result leads to a reduction in the number of immunosuppressive cells, such as Tregs, within the tumor, and thus can alleviate or reverse immunosuppression in the tumor. In some aspects, such reduction in immunosuppressive cells can result in the activation and proliferation of intratumoral T cells, such as intratumoral CD8⁺ cytotoxic T cells or CD4⁺ helper T cells, that can eliminate tumor cells, and lead to reduction of tumor volume and/or elimination of the tumor. In some aspects, treatment in accordance with the provided embodiments can result in a reduction of intratumoral Tregs and/or an increase of intratumoral CD8⁺ to Treg ratio or intratumoral CD4⁺ to Treg ratio.

In some aspects, treatment in accordance with the methods and uses provided herein can result in a lasting or durable decrease in intratumoral Tregs. In some aspects, treatment in accordance with the methods and uses provided herein can result in a lasting or durable increase of intratumoral CD8⁺ to Treg ratio or intratumoral CD4⁺ to Treg ratio. In some embodiments, the level, strength or extent of systemic immunity can be measured by determining the intratumoral CD8⁺ to Treg ratio, and the systemic immunity against recurring tumors is increased or augmented if the intratumoral CD8⁺ to Treg ratio is increased after treatment as compared to before treatment. In some embodiments, the level, strength or extent of systemic immunity can be measured by determining the intratumoral CD4⁺ to Treg ratio, and the systemic immunity against recurring tumors is increased or augmented if the intratumoral CD4⁺ to Treg ratio is increased after treatment as compared to before treatment. In some embodiments, the level, strength or extent of systemic immunity can be measured by determining the intratumoral Treg to CD45⁺ ratio, and the systemic immunity against recurring tumors is increased or augmented if the intratumoral Treg to CD45⁺ ratio is decreased after treatment as compared to before treatment. In some aspects, such increases or decreases can last for at or about 3, 4, 5, 6, 7, 8, 9, 10, 11, 12, 13, 14 days or 3, 4, 5, 6, 7 or 8 weeks or longer.

In some aspects, the level, strength or extent of systemic immunity can be measured by a CTL activity assay using splenocytes or peripheral blood cells or bone marrow cells or lymph node cells. In some embodiments, the cells are collected from the subject between day 4 and day 28 after illumination of the first tumor in the subject.

In some aspects, the level, strength or extent of systemic immunity can be measured by an intratumoral T cell exhaustion assay using T cells collected from the first tumor or a metastatic tumor cells mass or an invasive tumor cell mass. In some embodiments, the cells are collected from the subject between day 4 and day 28 after illumination of the first tumor in the subject.

In some aspects, the level, strength or extent of systemic immunity can be measured by an intratumoral effector T cell expansion assay using T cells collected from the first tumor or a metastatic tumor cells mass or an invasive tumor cell mass. In some embodiments, the cells are collected from the subject between day 4 and day 28 after illumination of the first tumor in the subject.

In some aspects, the level, strength or extent of systemic immunity can be measured by a T cell receptor diversity assay using T cells collected from the first tumor or a metastatic tumor cells mass or an invasive tumor cell mass or the peripheral circulation. In some embodiments, the cells are collected from the subject between day 4 and day 28 after illumination of the first tumor in the subject.

In some aspects, the level, strength or extent of systemic immunity can be measured by determining the presence, number or frequency of regulatory T cells (Tregs) in the tumor and/or the ratio of intratumoral Treg cells to intratumoral CD8⁺ T cells or intratumoral CD4⁺ T cells from the first tumor or a metastatic tumor cell mass or an invasive tumor cell mass. In some embodiments, the cells are collected from the subject between day 4 and day 28 after illumination of the first tumor in the subject.

In some embodiments, any of the above assays can be used in combination.

### III. CONJUGATES AND COMPOSITIONS FOR USE WITH THE METHODS

In some aspects, provided are conjugates comprising a phthalocyanine dye linked to a targeting molecule, compositions and combinations, for example, comprising a conjugate comprising a phthalocyanine dye linked to a targeting molecule, such as an antibody or an antigen-binding fragment thereof, that binds to CD25 protein, but does not block, such as does not substantially block, binding of IL-2 to CD25. In some aspects, the provided compositions or combinations comprise an immune checkpoint inhibitor, such as an anti-PD-1 antibody. In some aspects, provided are conjugates, compositions and combinations for use in a method of treatment or in a treatment regimen in accordance with the provided methods and uses, or in the manufacture of a medicament for the treatment of a cancer or a tumor. In some aspects, the treatment comprises a combination treatment. In some aspects, provided are compositions and combinations for use in accordance with the provided methods and uses.

The methods and uses provided herein employ a conjugate that comprises a targeting molecule that binds to CD25, such as an anti-CD25 antibody or an antigen-binding fragment that binds to, such as specifically binds to, CD25, but does not block, such as does not substantially block, IL-2 from also binding CD25 and/or does not interfere with, such as does not substantially interfere with, IL-2 mediated signaling via interaction with CD25 (i.e., IL-2 non-blocking anti-CD25 antibody). CD25 can be expressed on activated T cells, including CD8+ cells, CD4+FoxP3+ regulatory T cells, activated B cells, some thymocytes, myeloid precursors, and oligodendrocytes. CD25 is also known as interleukin 2 receptor alpha chain (IL2RA), IDDM10, IL2R, TCGFR, p55 or IMD41.

In some aspects, “IL-2 non-blocking antibody” or “IL-2 non-blocking anti-CD25 antibody” includes anti-CD25 antibodies (e.g. anti-CD25 IL-2 non-blocking antibodies) that are capable of specific binding to the CD25 subunit of the IL-2 receptor without blocking the binding of IL-2 to CD25 or signaling of IL-2 via CD25. In some aspects, the IL-2 non-blocking anti-CD25 antibodies allow at least 50% of IL-2 signaling in response to IL-2 binding to CD25 compared to the level of signaling in the absence of the anti-CD25 antibody. In some aspects, the IL-2 non-blocking anti-CD25 antibodies allow at least 75% of IL-2 signaling in response to IL-2 binding to CD25 compared to the level of signaling in the absence of the anti-CD25 antibody.

In some aspects, “IL-2 non-blocking”, in some cases with reference to “non-blocking,” “does not block,” or grammatical variation thereof, in some cases with respect to the non-blocking of IL-2 binding to CD25 in the presence of the anti-CD25 antibody, include instances in which the anti-CD25 antibody or antigen-binding fragment inhibits less than 50% of IL-2 signaling compared to IL-2 signaling in the absence of the antibodies. In some aspects, the anti-CD25 antibody or antigen-binding fragment inhibits less than about 40%, 35%, 30%, such as less than about 25% of IL-2 signaling compared to IL-2 signaling in the absence of the anti-CD25 antibodies. IL-2 non-blocking anti-CD25 antibodies can bind to CD25 without interfering with IL-2 binding to CD25, or without substantially interfering with IL-2 binding to CD25. In some aspects, an IL-2 non-blocking anti-CD25 antibody is alternatively referred to as an anti-CD25 antibody that “does not inhibit the binding of interleukin 2 to CD25” or as an anti-CD25 antibody that “does not inhibit the signaling of IL-2,” or “an antibody or antigen-binding fragment that specifically binds a CD25 without substantially blocking or interfering with IL-2 signaling.”

In some aspects, some anti-CD25 antibodies may allow binding of IL-2 to CD25, but still block signaling via the CD25 receptor. In some aspects, such antibodies are not considered to be IL-2 non-blocking. In some embodiments, the IL-2 non-blocking anti-CD25 antibodies allow binding of IL-2 to CD25 to facilitate at least 50% of the level of signaling via the CD25 receptor compared to the signaling in the absence of the IL-2 non-blocking anti-CD25 antibodies.

In some aspects, IL-2 signaling via CD25, can be assessed or measured by any known methods to assess or measure cell signaling, for example, by phosphorylation assays, binding assays, reporter assays, T cell activation assays, in vitro effector assays, in vitro antibody-dependent cell-mediated cytotoxicity assays (ADCC assays), in vitro Antibody-dependent cell-mediated phagocytosis (ADCP), cytokine secretion assays, target cell killing assays or model animal experiments. In some aspects, exemplary methods to assess IL-2 signaling via CD25 include, but is not limited to, any described in, for example, Rubin et al. (1985) Hybridoma 4(2) 91-102, Van Assche et al., Gut. 2006 November; 55(11): 1568-1574; Martin et al., J Immunol Jul. 15, 2010, 185 (2) 1311-1320, WO2019175223, WO2019175220, WO2019175222, WO2019175224, WO2019175216, WO2019175217, WO2019175226, WO2019175215, U.S. Ser. No. 10/745,485, US20210047420, U.S. Ser. No. 10/738,125, US20210009703, US20210040221, US20210009704, US20200407454, and US20210009699. In some aspects, comparison of IL-2 signaling in the presence and absence of the anti-CD25 antibody agent can occur under the same or substantially the same conditions.

In some embodiments, IL-2 signaling can be determined by measuring by the levels of phosphorylated STAT5 protein in cells, using a standard STAT5 phosphorylation assay. For example a STAT5 phosphorylation assay to measure IL-2 signaling may involve culturing PMBC cells in the presence of particular concentration(s) of the anti-CD25 antibody and then adding varying concentrations of IL-2 (for example, serial dilution of IL-2 concentrations). Cells may then be permeabilized and levels of STAT5 protein can then be measured with a fluorescent labelled antibody to a phosphorylated STAT5 peptide analyzed by flow cytometry. The percentage blocking of IL-2 signaling can be calculated as follows: % blocking=100×[(% STAT5⁺ cells no antibody treatment−% STAT5⁺ cells antibody treatment)/(% Stat5⁺ cells no antibody treatment)].

In some embodiments, the targeting molecule can be any IL-2 non-blocking anti-CD25 antibody. Non-limiting, exemplary IL-2 non-blocking anti-CD25 antibodies with SEQ ID NOS corresponding to the amino acid sequences for the variable heavy chain (V_{H}), variable light chain (V_{L}), and exemplary corresponding complementarity determining regions (CDRs), for example, according to Chothia numbering, are listed in Table 1. In some embodiments, the targeting molecule is an IL-2 non-blocking anti-CD25 antibody listed in Table 1, or an antigen-binding fragment thereof. In some embodiments, the targeting molecule is an CL-2 non-blocking anti-CD25 antibody, or an antigen-binding fragment, which competes with any one or more of the antibodies listed in Table 1 for the same or an overlapping epitope. In some embodiments, the targeting molecule is an IL-2 non-blocking anti-CD25 antibody, or antigen fragment, that binds a non-overlapping epitope with one or more of the antibodies listed in Table 1.

TABLE 1
Sequence identifier (SEQ ID NO) for
Exemplary Anti-CD25 Antibodies

| | Heavy Chain | | | | Light Chain | | | |
|---|---|---|---|---|---|---|---|---|
| Clone | | CDR- | CDR- | CDR- | | CDR- | CDR- | CDR- |
| # | V_{H} | H1 | H2 | H3 | V_{L} | L1 | L2 | L3 |
| 7G7B6 | 1 | 20 | 21 | 22 | 2 | 23 | 24 | 25 |
| MA251 | 3 | 26 | 27 | 28 | 4 | 29 | 24 | 30 |
| A | 5 | 31 | 32 | 33 | 6 | 34 | 35 | 36 |
| B | 7 | 37 | 38 | 39 | 8 | 40 | 41 | 42 |
| C | 9 | 20 | 21 | 22 | 11 | 23 | 24 | 25 |
| D | 9 | 20 | 21 | 22 | 12 | 23 | 24 | 25 |
| E | 10 | 20 | 21 | 22 | 11 | 23 | 24 | 25 |
| F | 10 | 20 | 21 | 22 | 12 | 23 | 24 | 25 |
| G | 13 | 26 | 27 | 28 | 16 | 29 | 24 | 30 |
| H | 13 | 26 | 27 | 28 | 17 | 29 | 24 | 30 |
| I | 13 | 26 | 27 | 28 | 18 | 29 | 24 | 30 |
| J | 13 | 26 | 27 | 28 | 19 | 29 | 24 | 30 |
| K | 14 | 26 | 27 | 28 | 16 | 29 | 24 | 30 |
| L | 14 | 26 | 27 | 28 | 17 | 29 | 24 | 30 |
| M | 14 | 26 | 27 | 28 | 18 | 29 | 24 | 30 |
| N | 14 | 26 | 27 | 28 | 19 | 29 | 24 | 30 |
| O | 15 | 26 | 27 | 28 | 16 | 29 | 24 | 30 |
| P | 15 | 26 | 27 | 28 | 17 | 29 | 24 | 30 |
| Q | 15 | 26 | 27 | 28 | 18 | 29 | 24 | 30 |
| R | 15 | 26 | 27 | 28 | 19 | 29 | 24 | 30 |

In some of any embodiments, the targeting molecule is an antibody or antigen-binding fragment thereof or includes an antigen-binding fragment. In some of any embodiments, the antibody is an IL-2 non-blocking anti-CD25 antibody. In some of any of the embodiments, the IL-2 non-blocking anti-CD25 antibody includes a functional Fc region. In some of any of the embodiments, the anti-CD25 antibody includes a full-length Fc region. In some embodiments, the IL-2 non-blocking anti-CD25 antibody is an antibody fragment. In any of the provided embodiments, the antibody or antibody fragment can be humanized according to known methods. In some embodiments, the antibody or antibody fragment is a human, chimeric or humanized antibody.

In some of any of the embodiments, the IL-2 non-blocking anti-CD25 antibody is an antibody listed in Table 1, or a biosimilar, interchangeable, biobetter, copy biological or biogeneric thereof or a fragment thereof.

In some of any of the embodiments, the IL-2 non-blocking anti-CD25 antibody or fragment thereof comprises a V_{H} comprising a CDR-H1, the CDR-H2, and the CDR-H3, and a V_{L} comprising the CDR-L1, the CDR-L2, and the CDR-L3, according to an antibody numbering scheme (e.g., Kabat, Chothia, Contact, IMGT, Aho, or AbM numbering scheme), of an antibody with a V_{H} and a V_{L} listed in Table 1, for example, in each row of Table 1. In some of any of the embodiments, the IL-2 non-blocking anti-CD25 antibody or fragment thereof comprises a V_{H} comprising the CDR-H1, the CDR-H2, and the CDR-H3, and a V_{L} comprising the CDR-L1, the CDR-L2, and the CDR-L3, of an antibody listed in Table 1. In some of any of the embodiments, the IL-2 non-blocking anti-CD25 antibody or fragment thereof comprises a V_{H} comprising a CDR-H1, a CDR-H2 and a CDR-H3 of an antibody listed in Table 1, and a V_{L} comprising a CDR-L1, a CDR-L2 and a CDR-L3 of an antibody listed in Table 1. In some embodiments, the IL-2 non-blocking anti-CD25 antibody or fragment thereof, comprises a V_{H} and a V_{L} region set forth in the SEQ ID NOS: listed in each row of Table 1 below, or an antibody comprising a V_{H} and a V_{L} sequence that has at least 90%, 91%, 92%, 93%, 94%, 95%, 96%, 97%, 98%, or 99% sequence identity to the V_{H} and the sequence set forth in the SEQ ID NOS: listed in each row of Table 1. In some of any of the embodiments, the IL-2 non-blocking anti-CD25 antibody comprises a V_{H} and a V_{L} comprising the V_{H} and the V_{L}, respectively, of an antibody listed in Table 1, for example, in each row of Table 1.

In some of any of the provided embodiments, exemplary IL-2 non-blocking anti-CD25 antibody of the conjugate includes any known IL-2 non-blocking anti-CD25 antibody, a humanized form thereof, and/or an antigen-binding fragment thereof. In some aspects, exemplary the IL-2 non-blocking anti-CD25 antibody of the conjugate includes, but is not limited to, any described in, for example, Rubin et al. (1985) Hybridoma 4(2) 91-102, Van Assche et al., Gut. 2006 November; 55(11): 1568-1574; Martin et al., J Immunol Jul. 15, 2010, 185 (2) 1311-1320, WO2019175223, WO2019175220, WO2019175222, WO2019175224, WO2019175216, WO2019175217, WO2019175226, WO2019175215, U.S. Ser. No. 10/745,485, US20210047420, U.S. Ser. No. 10/738,125, US20210009703, US20210040221, US20210009704, US20200407454, and US20210009699, a humanized form thereof, and/or an antigen-binding fragment thereof.

In some of any of the provided embodiments, the IL-2 non-blocking anti-CD25 antibody is 7G7B6 (Rubin et al. (1985) Hybridoma 4(2) 91-102), or a humanized 7G7B6 antibody, a fragment thereof or is derived from 7G7B6. In some of any of the embodiments, the IL-2 non-blocking anti-CD25 antibody is 7G7B6, or a humanized form thereof (humanized 7G7B6), comprising an Fc region. In some of any of the embodiments, the IL-2 non-blocking anti-CD25 antibody is 7G7B6, or humanized 7G7B6, comprising an Fc region that is engineered to exhibit antibody-dependent cellular cytotoxicity (ADCC) activity or exhibit enhanced ADCC activity. In some of any of the embodiments, the IL-2 non-blocking anti-CD25 antibody is 7G7B6, or humanized 7G7B6, comprising a full-length Fc region. In some of any of the embodiments, the IL-2 non-blocking anti-CD25 antibody is 7G7B6, or humanized 7G7B6, comprising an Fc region that is engineered to exhibit ADCC activity or exhibit enhanced ADCC activity. In some of any of the embodiments, the IL-2 non-blocking anti-CD25 antibody is 7G7B6, or humanized 7G7B6, comprising an Fc region that does not exhibit ADCC activity, does not exhibit substantial ADCC activity, or exhibits reduced ADCC activity.

In some of any of the provided embodiments, the IL-2 non-blocking anti-CD25 antibody is MA251 (see, e.g., Van Assche et al., Gut. 2006 November; 55(11): 1568-1574; Martin et al., J Immunol Jul. 15, 2010, 185 (2) 1311-1320), or a humanized form thereof (humanized MA251), a fragment thereof or is derived from MA251. In some of any of the embodiments, the IL-2 non-blocking anti-CD25 antibody is MA251, or humanized MA251, comprising an Fc region. In some of any of the embodiments, the IL-2 non-blocking anti-CD25 antibody is MA251, or humanized MA251, comprising an Fc region that is engineered to exhibit antibody-dependent cellular cytotoxicity (ADCC) activity or exhibit enhanced ADCC activity. In some of any of the embodiments, the IL-2 non-blocking anti-CD25 antibody is MA251, or humanized MA251, comprising a full-length Fc region. In some of any of the embodiments, the IL-2 non-blocking anti-CD25 antibody is MA251, or humanized MA251, comprising an Fc region that is engineered to exhibit ADCC activity or exhibit enhanced ADCC activity. In some of any of the embodiments, the IL-2 non-blocking anti-CD25 antibody is MA251, or humanized MA251, comprising an Fc region that does not exhibit ADCC activity, does not exhibit substantial ADCC activity, or exhibits reduced ADCC activity.

In some of any of the provided embodiments, the IL-2 non-blocking anti-CD25 antibody is “clone A” (as described in Table 1), a fragment thereof or is derived from “clone A”. In some of any of the embodiments, the IL-2 non-blocking anti-CD25 antibody is clone A comprising an Fc region. In some of any of the embodiments, the IL-2 non-blocking anti-CD25 antibody is “clone A” comprising an Fc region that is engineered to exhibit antibody-dependent cellular cytotoxicity (ADCC) activity or exhibit enhanced ADCC activity. In some of any of the embodiments, the IL-2 non-blocking anti-CD25 antibody is “clone A” comprising a full-length Fc region. In some of any of the embodiments, the IL-2 non-blocking anti-CD25 antibody is “clone A” comprising an Fc region that is engineered to exhibit ADCC activity or exhibit enhanced ADCC activity. In some of any of the embodiments, the IL-2 non-blocking anti-CD25 antibody is “clone A” comprising an Fc region that does not exhibit ADCC activity, does not exhibit substantial ADCC activity, or exhibit reduced ADCC activity.

In some of any of the provided embodiments, the IL-2 non-blocking anti-CD25 antibody is “clone B” (as described in Table 1), a fragment thereof or is derived from “clone B”. In some of any of the embodiments, the IL-2 non-blocking anti-CD25 antibody is “clone B” comprising an Fc region. In some of any of the embodiments, the IL-2 non-blocking anti-CD25 antibody is “clone B” comprising an Fc region that is engineered to exhibit antibody-dependent cellular cytotoxicity (ADCC) activity or exhibit enhanced ADCC activity. In some of any of the embodiments, the IL-2 non-blocking anti-CD25 antibody is “clone B” comprising a full-length Fc region. In some of any of the embodiments, the IL-2 non-blocking anti-CD25 antibody is “clone B” comprising an Fc region that is engineered to exhibit ADCC activity or exhibit enhanced ADCC activity. In some of any of the embodiments, the IL-2 non-blocking anti-CD25 antibody is “clone B” comprising an Fc region that does not exhibit ADCC activity, does not exhibit substantial ADCC activity, or exhibits reduced ADCC activity.

In some embodiments, the targeting molecule can be an antibody or antibody fragment that includes the “complementarity-determining regions” or “CDRs” of an IL-2 non-blocking anti-CD25 antibody, such as any of the described antibodies or antigen-binding fragment thereof, for example in Table 1. The CDRs are typically responsible for binding to an epitope of an antigen. The precise amino acid sequence boundaries of a given CDR can be readily determined using any of a number of well-known schemes. Exemplary numbering schemes include Kabat, Chothia, Contact, IMGT, Aho, and AbM numbering schemes. The boundaries of a given CDR may vary depending on the scheme used for identification. For example, the Kabat scheme is based on structural alignments, while the Chothia scheme is based on structural information. Numbering for both the Kabat and Chothia schemes is based upon the most common antibody region sequence lengths, with insertions accommodated by insertion letters, for example, “30a,” and deletions appearing in some antibodies. The two schemes place certain insertions and deletions (“indels”) at different positions, resulting in differential numbering. The Contact scheme is based on analysis of complex crystal structures and is similar in many respects to the Chothia numbering scheme. The AbM scheme is a compromise between Kabat and Chothia definitions based on that used by Oxford Molecular's AbM antibody modeling software. Any antibody numbering scheme can be used to identify CDR regions of the antibodies and conjugates described herein and used in the provided methods and uses.

The CDRs of each chain are typically referred to as CDR1, CDR2, and CDR3, numbered sequentially starting from the N-terminus, and are also generally identified by the chain in which the particular CDR is located. Thus, a heavy chain variable region (V_{H}) CDR3 (also called CDR-H3) is located in the variable domain of the heavy chain of the antibody in which it is found, whereas a light chain variable region (V_{L}) CDR1 (also called CDR-L1) is the CDR1 from the variable domain of the light chain of the antibody in which it is found. Antibodies with different specificities, such as different combining sites for different antigens, have different CDRs. Although it is the CDRs that vary from antibody to antibody, only a limited number of amino acid positions within the CDRs are directly involved in antigen binding. These positions within the CDRs are called specificity determining residues (SDRs). In some embodiments, the targeting molecule includes CDRs from 7G7B6, MA251, or “clone A” or “clone B,” such as those described in Table 1, according to the Chothia numbering scheme. In some embodiments, the targeting molecule includes CDRs from 7G7B6, MA251, or “clone A” or “clone B” according to a different numbering scheme. In some embodiments, the antibody of the conjugate is a biosimilar, interchangeable or biobetter of any of the anti-CD25 antibody described herein, e.g., 7G7B6, MA251, “clone A, or “clone B”, or an antigen-binding fragment thereof. Such antibodies also include copy biologicals and biogenerics of any of the anti-CD25 antibody described herein, or an antigen-binding fragment thereof.

In some embodiments of the methods and uses provided herein, an IL-2 non-blocking anti-CD25 antibody comprises a functional Fc region. In some embodiments of the methods and uses provided herein, an IL-2 non-blocking anti-CD25 antibody comprises a full-length Fc region. In some embodiments, an IL-2 non-blocking anti-CD25 antibody comprises an IgG1 Fc region. In some embodiments, an IL-2 non-blocking anti-CD25 antibody comprises an IgG2 Fc region. In some embodiments, the IgG2 Fc region is an IgG2a Fc region. In some embodiments, the IgG2 Fc region is an IgG2a/b Fc region. In some embodiments, the IgG2 Fc region is an IgG2a Fc region. In some embodiments of the methods and uses provided herein, an IL-2 non-blocking anti-CD25 antibody comprises an IgG3 Fc region. In some embodiments of the methods and uses provided herein, an IL-2 non-blocking anti-CD25 antibody comprises an IgG4 Fc region. In some embodiments, the Fc region is modified to modulate effector functions of the antibody portion of the conjugate. Any of such modifications, such as those described in Wang et al., (2018) Protein Cell. 9(1): 63-73, are contemplated for the antibodies, antibody fragments, and conjugates described herein.

In some embodiments of the methods and uses provided herein, an IL-2 non-blocking anti-CD25 antibody does not comprise a functional Fc region. In some of such examples, the IL-2 non-blocking antibody does not contain an Fc region or comprises an Fc region that has been modified such that the Fc region does not bind an Fc receptor and/or does not elicit substantial Fc effector function (e.g., ADCC, ADCP, and/or CDC). In some of such embodiments, the IL-2 non-blocking antibody comprises an Fc receptor that contains an amino acid substitution to eliminate the glycosylation site at a position corresponding to position 297 of the heavy chain based on EU numbering described by Edelman et al., (1969) Proc Natl Acad Sci USA. 63(1):78-85. For example, the IL-2 non-blocking antibody can comprise an Fc receptor that contains an asparagine to glutamine substitution at or corresponding to position 297 (N297Q) with respect to EU numbering of the antibody sequence, an asparagine to alanine substitution at or corresponding to position 297 (N297A) with respect to EU numbering of the antibody sequence, or an asparagine to glycine substitution at or corresponding to position 297 (N297G) with respect to EU numbering of the antibody sequence.

In some embodiments of the methods and uses provided herein, an IL-2 non-blocking anti-CD25 antibody comprises an Fc region that exhibits enhanced Fc-mediated effector function, such as ADCC, ADCP, and/or CDC activity, and/or exhibits preferential binding to the Fc gamma receptor. In some embodiments, the IL-2 non-blocking anti-CD25 antibody exhibits enhanced function due to increased Fc receptor engagement. In some embodiments, the Fc region contains one or more of the following mutations: a serine to aspartic acid substitution at position 239 (S239D), an alanine to leucine substitution at position 330 (A330L), an isoleucine to glutamic acid substitution at position 332 (I332E), a glutamic acid to alanine substitution at position 333 (E333A), a lysine to alanine substitution at position 334 (K334A), an arginine to alanine substitution at position 255 (S255A), a threonine to alanine substitution at position 256 (T256A), a serine to alanine substitution at position 267 (S267A), a serine to alanine substitution at position 298 (S298A), an asparagine to serine substitution at position 325 (N325S), a leucine to phenylalanine at position 328 (L328F), an alanine to leucine substitution at position 330 (A330L), an isoleucine to glutamic acid substitution at position 333 (E333A), a glutamic acid to alanine substitution at position 333 (E333A), a lysine to alanine substitution at position 334 (K334), and/or an alanine to glutamine substitution at position 378 (A378Q) with respect to EU numbering of the antibody heavy chain. In some embodiments, the Fc region contains a serine to alanine substitution at position 298 and a lysine to alanine substitution at position 334 (S298A/K334) with respect to EU numbering of the antibody heavy chain. In some embodiments, the Fc region contains a glutamic acid to alanine substitution at position 333 and a lysine to alanine substitution at position 334 (E33A/K334) with respect to EU numbering of the antibody heavy chain. In some embodiments, the Fc region contains an arginine to alanine substitution at position 255 and a serine to alanine substitution at position 267 (R255A/S267A) with respect to EU numbering of the antibody heavy chain. In some embodiments, the FC region contains a threonine to alanine substitution at position 256 (T256A) with respect to EU numbering of the antibody heavy chain. In some embodiments, the Fc region contains a lysine to alanine substitution at position 334 and an alanine to glutamine substitution at position 378 (K334/A378) with respect to EU numbering of the antibody heavy chain. In some embodiments, the Fc region contains a serine to aspartic acid substitution at position 239, an alanine to leucine substitution at position 330, and an isoleucine to glutamic acid substitution at position 332 (S239D/A330L/I332E) with respect to EU numbering of the antibody heavy chain. In some embodiments, the Fc region contains a glycine to alanine substitution at position 236, a serine to aspartic substitution at position 239, and an isoleucine to glutamic acid substitution at position 332 (S239D/A330L/I332E) with respect to EU numbering of the antibody heavy chain. In some embodiments, the Fc region contains an arginine to serine substitution at position 325 and a leucine to phenylalanine substitution at position 328 (N325S/L328F) with respect to EU numbering of the antibody heavy chain.

The conjugates used in the methods and uses provided herein include a phthalocyanine dye. In some embodiments of the methods and uses provided herein, a phthalocyanine dye is a phthalocyanine dye with a silicon coordinating metal (Si-phthalocyanine dye). In any of the embodiments, the phthalocyanine dye is a salt, stereoisomer, or tautomer of any of the dyes described herein. In any of the embodiments, the phthalocyanine dye is an ionic form of any of the dyes described herein.

In some embodiments, the phthalocyanine dye comprises the formula:

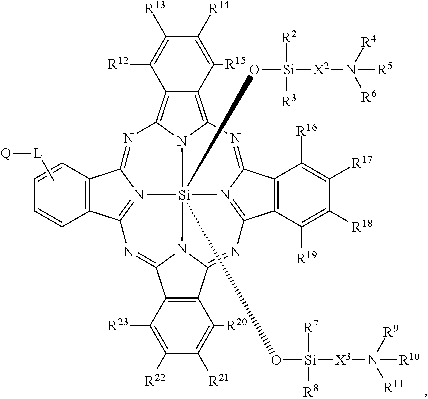

wherein:
L is a linker;
Q is a reactive group for attachment of the dye to the targeting molecule;
R², R³, R⁷, and R⁸ are each independently selected from among optionally substituted alkyl and optionally substituted aryl;
R⁴, R⁵, R⁶, R⁹, R¹⁰, and R¹¹ are each independently selected from among hydrogen, optionally substituted alkyl, optionally substituted alkanoyl, optionally substituted alkoxycarbonyl, optionally substituted alkylcarbamoyl, and a chelating ligand, wherein at least one of R⁴, R⁵, R⁶, R⁹, R¹⁰, and R¹¹ comprises a water-soluble group;
R¹², R¹³, R¹⁴, R¹⁵, R¹⁶, R¹⁷, R¹⁸, R¹⁹, R²⁰, R²¹, R²² and R²³ are each independently selected from among hydrogen, halogen, optionally substituted alkylthio, optionally substituted alkylamino and optionally substituted alkoxy; and
X² and X³ are each independently C₁-C₁₀ alkylene, optionally interrupted by a heteroatom.

In some embodiments, the phthalocyanine dye comprises the formula:

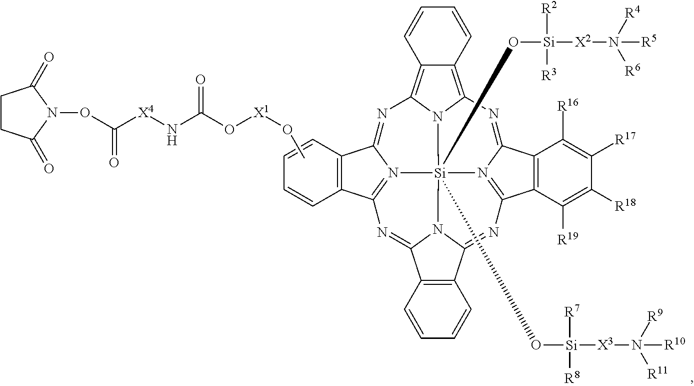

X¹ and X⁴ are each independently a C₁-C₁₀ alkylene optionally interrupted by a heteroatom;
R², R³, R⁷, and R⁸ are each independently selected from optionally substituted alkyl and optionally substituted aryl;
R⁴, R⁵, R⁶, R⁹, R¹⁰, and R¹¹ are each independently selected from among hydrogen, optionally substituted alkyl, optionally substituted alkanoyl, optionally substituted alkoxycarbonyl, optionally substituted alkylcarbamoyl, and a chelating ligand, wherein at least one of R⁴, R⁵, R⁶, R⁹, R¹⁰, and R¹¹ comprises a water-soluble group; and
R¹⁶, R¹⁷, R¹⁸ and R¹⁹ are each independently selected from among hydrogen, halogen, optionally substituted alkylthio, optionally substituted alkylamino and optionally substituted alkoxy.

In some embodiments the phthalocyanine dye is a Si-phthalocyanine dye that is IRDye 700DX (IR700). In some embodiments, the phthalocyanine dye containing the reactive group is IR700 NHS ester, such as IRDye 700DX NHS ester (LiCor 929-70010, 929-70011). In some embodiments, the dye is a compound having the following formula, or is a salt, ionic form, stereoisomer, or tautomer thereof:

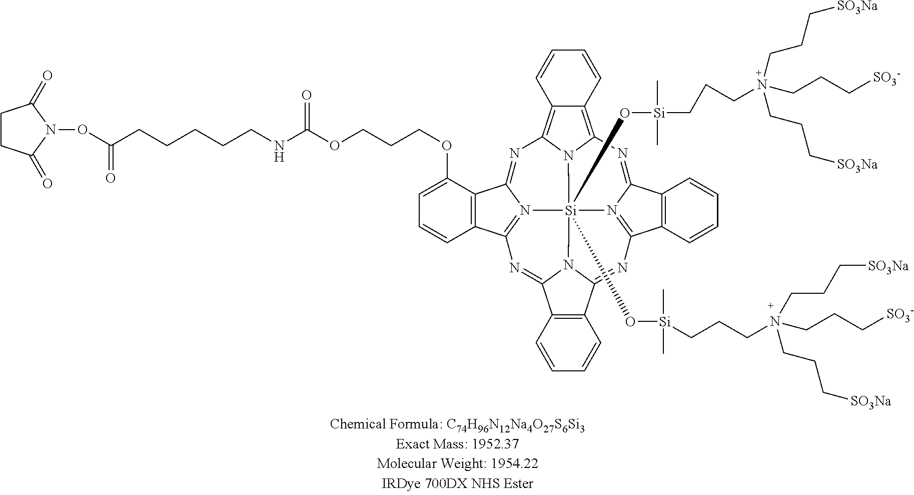

For purposes herein, the term “IR700,” “IRDye 700” or “IRDye 700DX” includes the above formula when the dye is conjugated such as to an antibody, e.g., via a reactive group.

In some embodiments the phthalocyanine dye is any dye described in WO 2021/207691. In some embodiments the dye is a silicon phthalocyanine dye described in WO 2021/207691.

In some embodiments, the phthalocyanine dye comprises Formula (X):

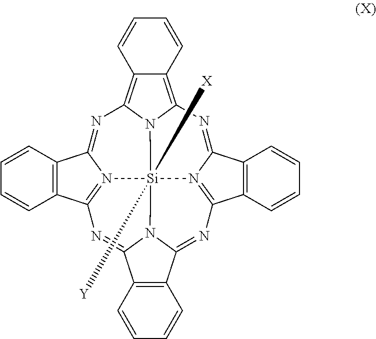

or a salt, ionic form, stereoisomer, or tautomer thereof, wherein:
X is

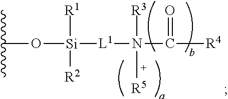

Y is

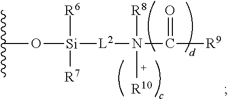

R¹ and R² are each independently optionally substituted alkyl, optionally substituted cycloalkyl, optionally substituted cycloalkylalkyl, optionally substituted heterocyclyl, optionally substituted heterocyclylalkyl, optionally substituted aryl, optionally substituted aralkyl, optionally substituted heteroaryl, or optionally substituted heteroaralkyl;
R³, R⁴ or R⁵ are selected from substituent group (a) or substituent group (b) wherein,
(a) R³ is hydrogen, -L³-H, -L³-A, or -L³-Z;
R⁴ is -L⁴-H, —(NH)ₘ-L⁴-A, —(NH)ₘ-L⁴-Z, —(O)ₘL⁴-A or —(O)ₘ-L⁴-Z;
R⁵ is -L⁵-H or -L⁵-A; and
(b) R³ is -L³-H, or -L³-A;
R⁴ is -L⁴-H, —(NH)ₘ-L⁴-A, or —(O)ₘ-L⁴-A; wherein R³ and R⁴ are connected with a bond to form a heterocyclyl substituted with -L⁴-A; and
R⁵ is -L⁵-H or -L⁵-A;
provided at least one of R³, R⁴ and R⁵ is a group containing A;
A is a reactive group capable of forming a covalent bond with a thiol, hydroxyl, carboxyl or amino group of a second moiety, or a protected form thereof or a reacted form thereof;
R⁶ and R⁷ are each independently optionally substituted alkyl, optionally substituted cycloalkyl, optionally substituted cycloalkylalkyl, optionally substituted heterocyclyl, optionally substituted heterocyclylalkyl, optionally substituted aryl, optionally substituted aralkyl, optionally substituted heteroaryl or optionally substituted heteroaralkyl;
R⁸, R⁹ or R¹⁰ are selected from substituent group (a) or substituent group (b) wherein,
(a) R⁸ is hydrogen, -L⁸-H or -L⁸-Z;
R⁹ is -L⁹-H, —(NH)ₙ-L⁹-Z or —(O)ₙ-L⁹-Z
R¹⁰ is -L¹⁰-Z; and
(b) R⁸ and R⁹ are connected with a bond to form a heterocyclyl substituted with -L⁹-Z and R¹⁰ is -L¹⁰-H or -L¹⁰-Z;
provided at least one of R⁸, R⁹ and R¹⁰ is a group containing Z;
Z is a water soluble group optionally substituted with A or L′-A;
L¹ and L² are each independently optionally substituted alkylene, optionally substituted heteroalkylene, optionally substituted alkenylene, optionally substituted heteroalkenylene, optionally substituted cycloalkyl, or optionally substituted heterocyclyl;
L³, L⁴, L⁵, L⁸, L⁹ and L¹⁰ are each independently optionally substituted alkylene, optionally substituted heteroalkylene, optionally substituted alkenylene, optionally substituted heteroalkenylene, optionally substituted cycloalkylene, optionally substituted heterocyclene, optionally substituted arylene, optionally substituted aralkylene, optionally substituted heteroaralkylene, or optionally substituted heteroarylene, where the carbon atom of the alkylene, heteroalkylene, alkenylene, heteroalkenylene, cycloalkylene, heterocyclene, arylene, aralkylene, heteroaralkylene, or optionally substituted heteroarylene is further optionally substituted with Z and each nitrogen atom of the heteroalkylene or heteroalkenylene is optionally substituted with one or two L′-Z;
L′ is each independently optionally substituted alkylene, optionally substituted heteroalkylene, optionally substituted alkenylene, optionally substituted heteroalkenylene, optionally substituted cycloalkylene, optionally substituted heterocyclene, optionally substituted arylene, optionally substituted aralkylene, optionally substituted heteroaralkylene, or optionally substituted heteroarylene;
a is 0 or 1;
b is 0 or 1;
c is 0 or 1;
d is 0 or 1;
m is 0 or 1;
n is 0 or 1;
provided that if b is 1, then a is 0;
if d is 1, then c is 0;
if m is 1, b is 1; and
if n is 1, c is 1.

In certain embodiments, the silicon phthalocyanine dye is selected from among the following formulae, or a salt, ionic form, stereoisomer, or tautomer thereof:

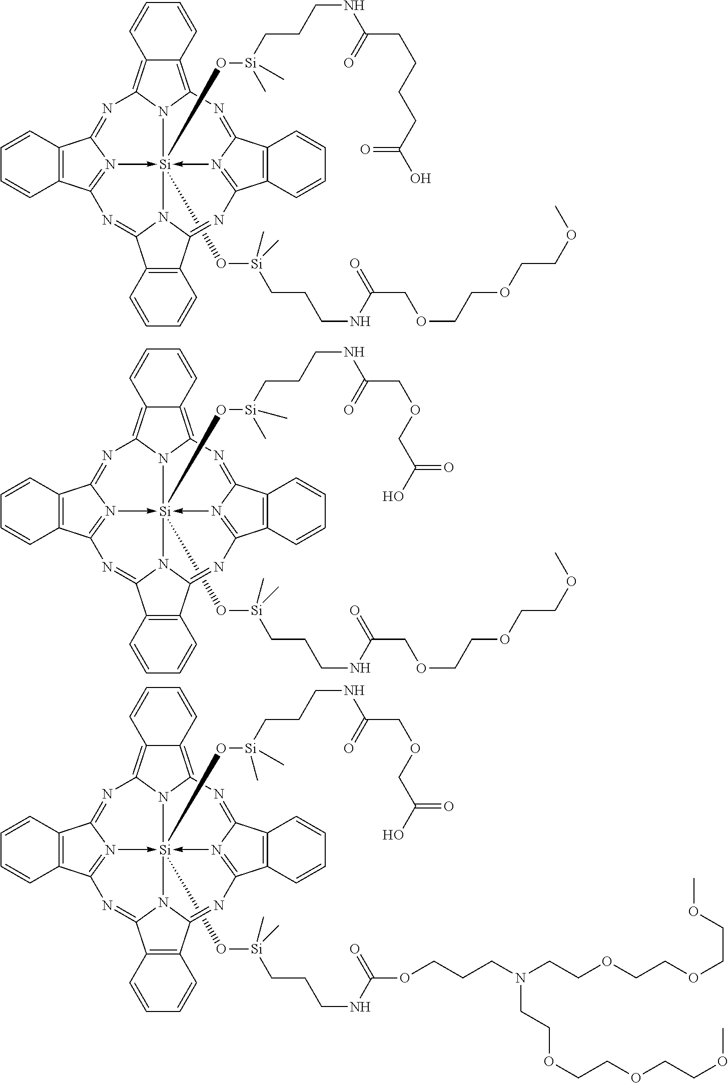

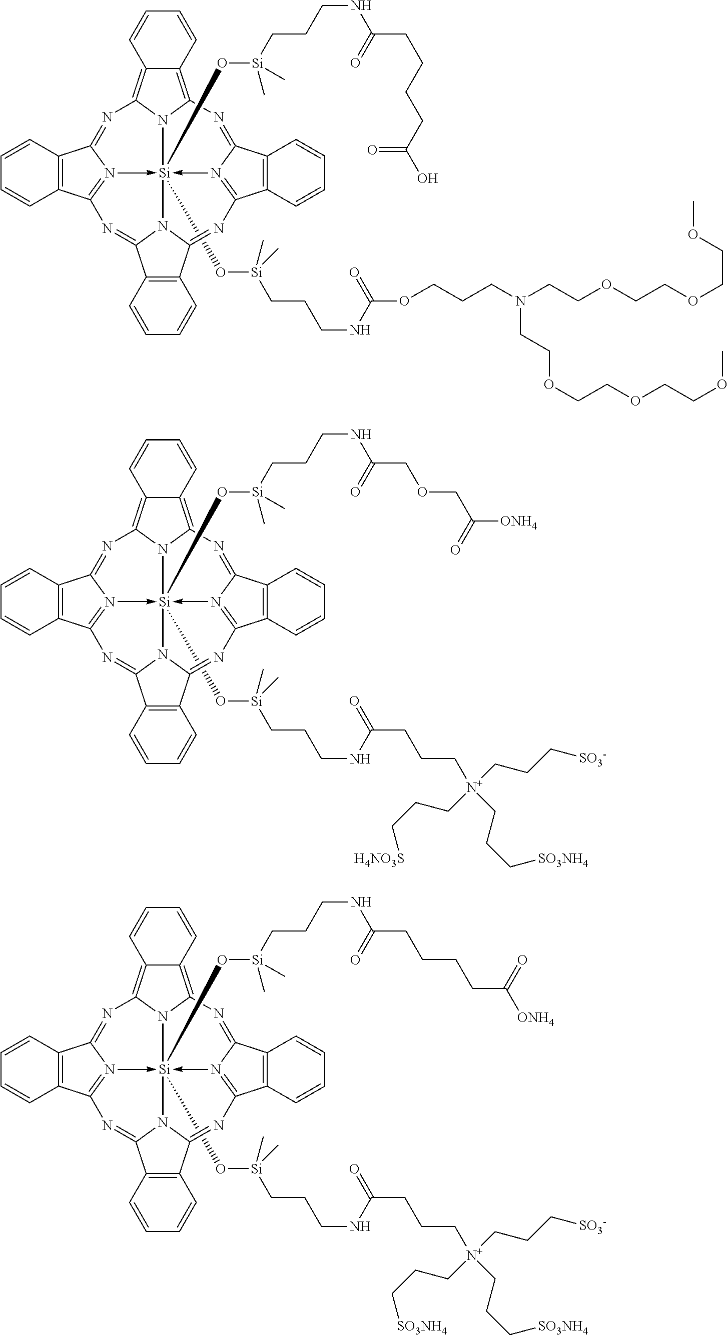

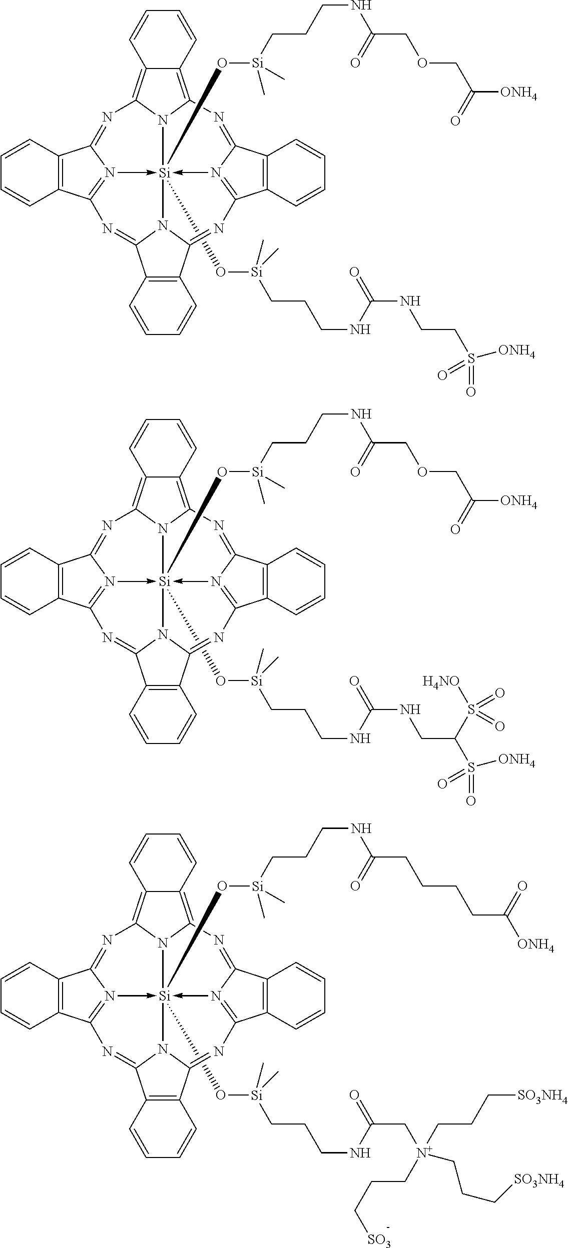

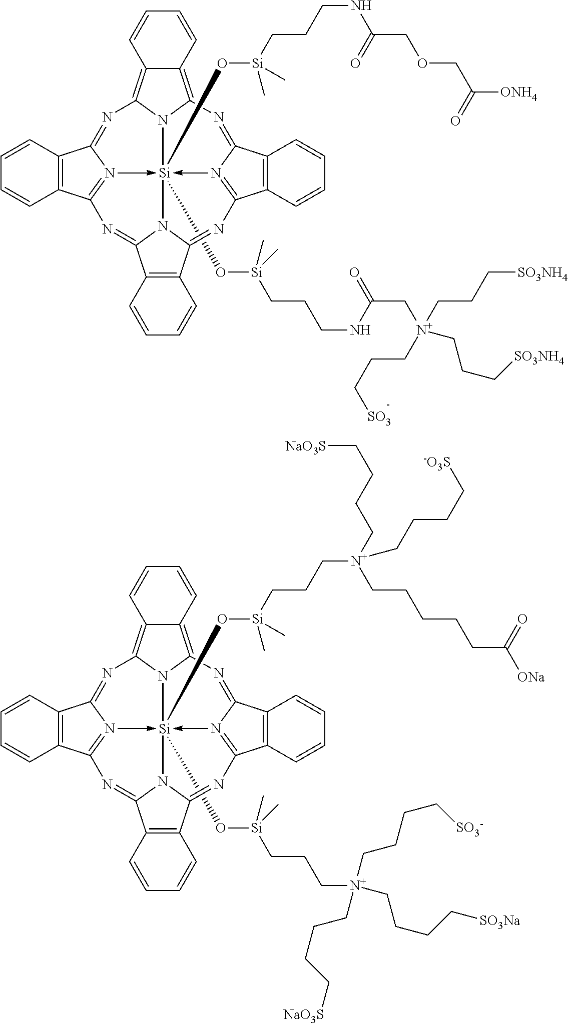

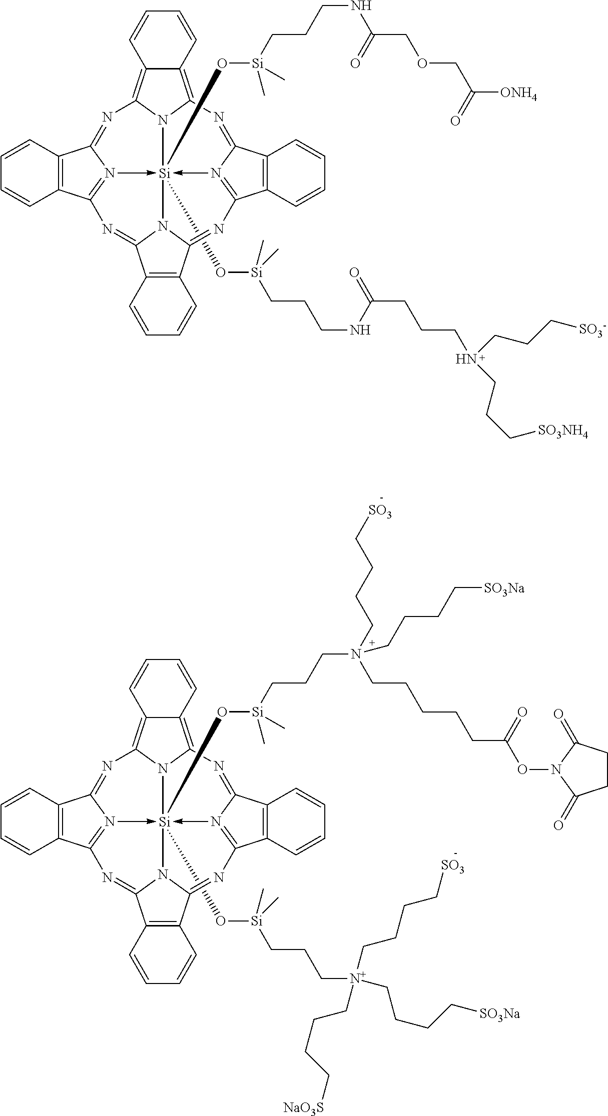

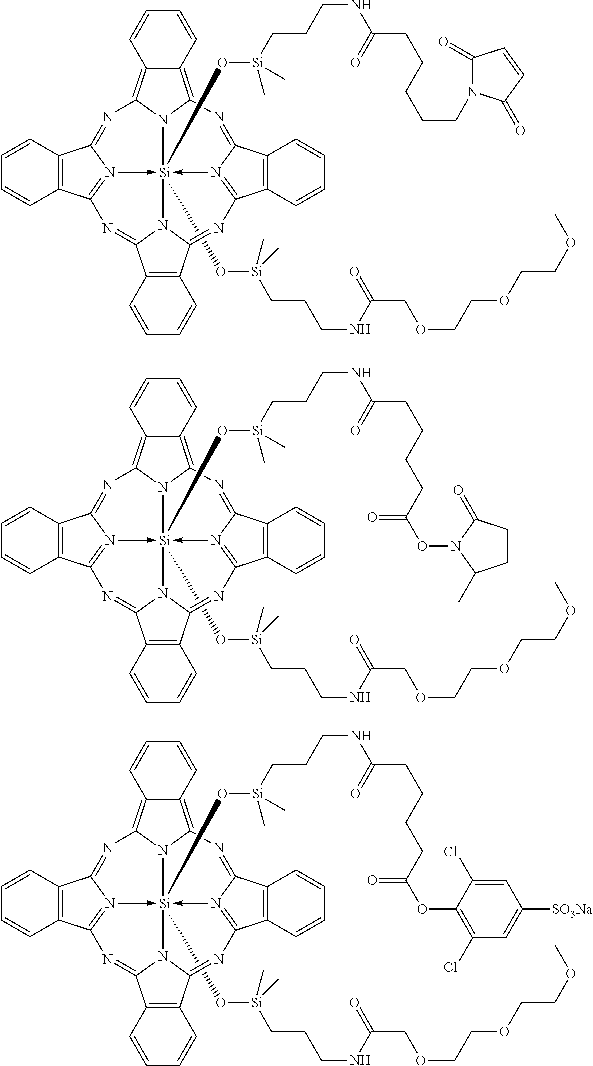

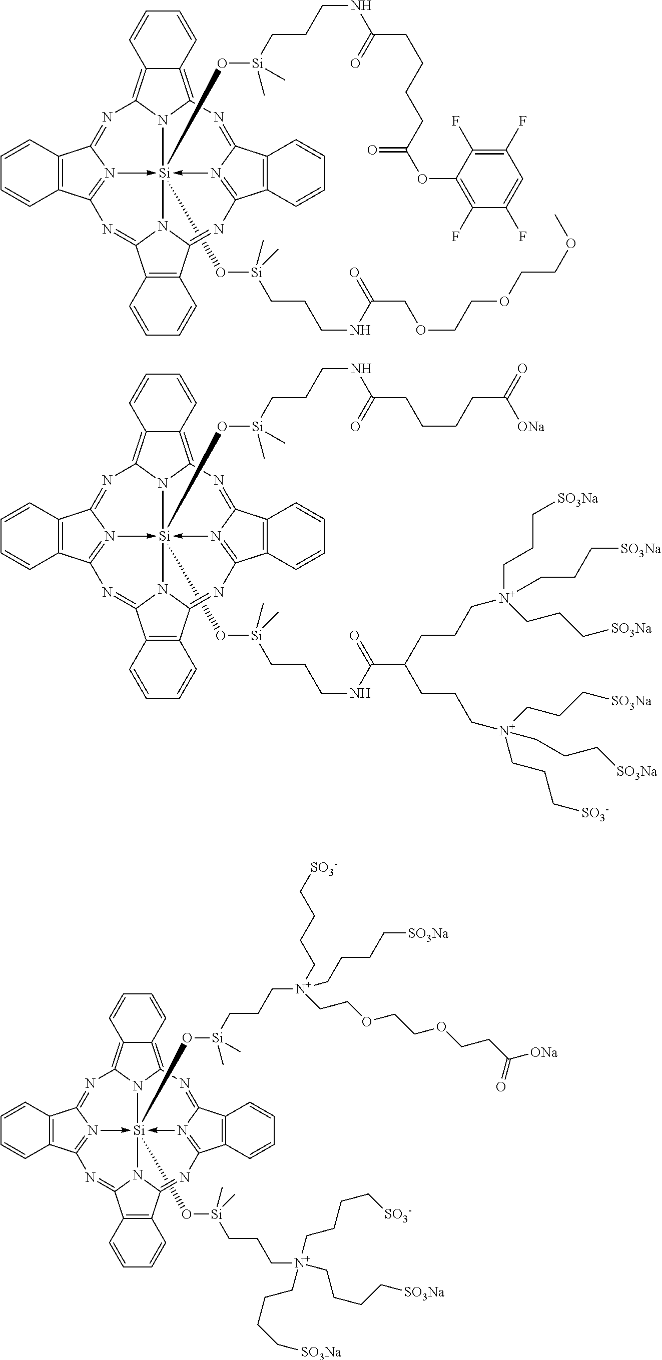

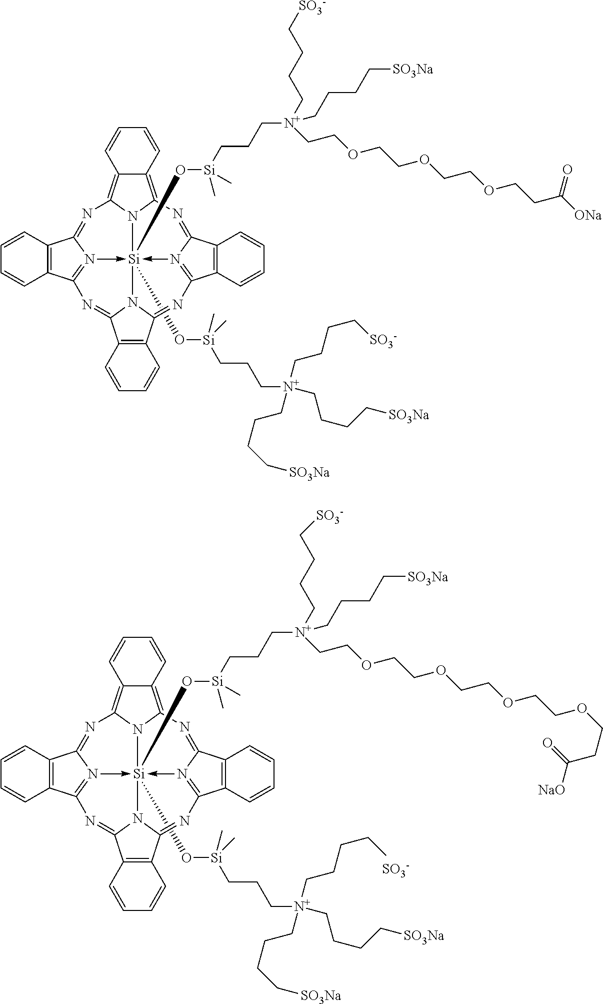

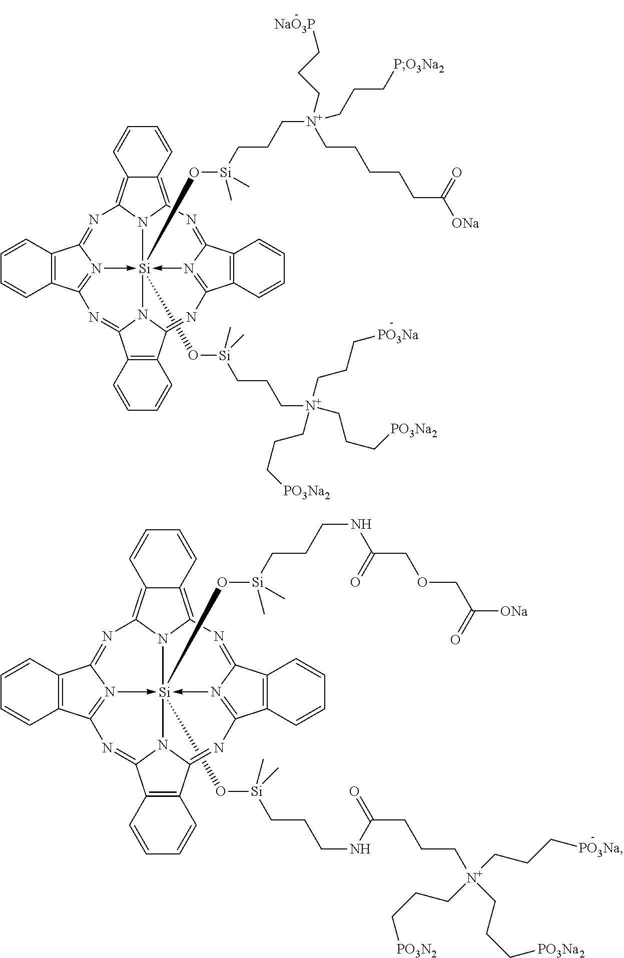

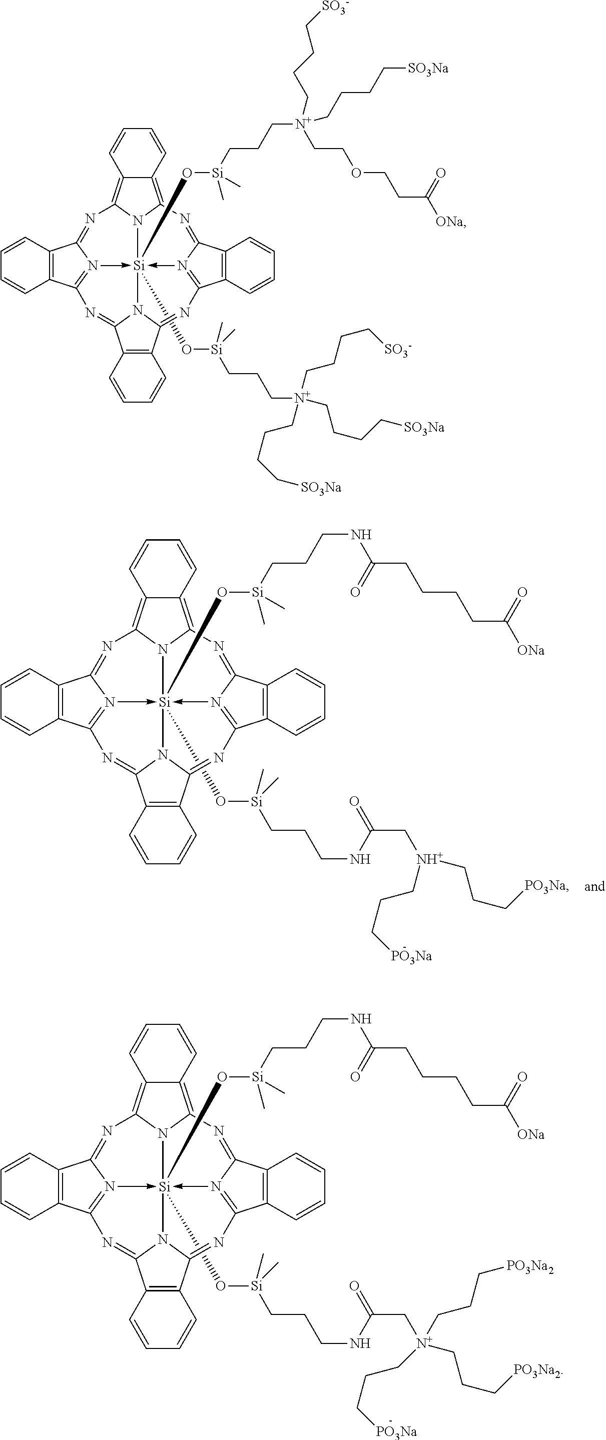

In certain embodiments, the silicon phthalocyanine dye is selected from among the following formulae, or a salt, ionic form, stereoisomer, or tautomer thereof:

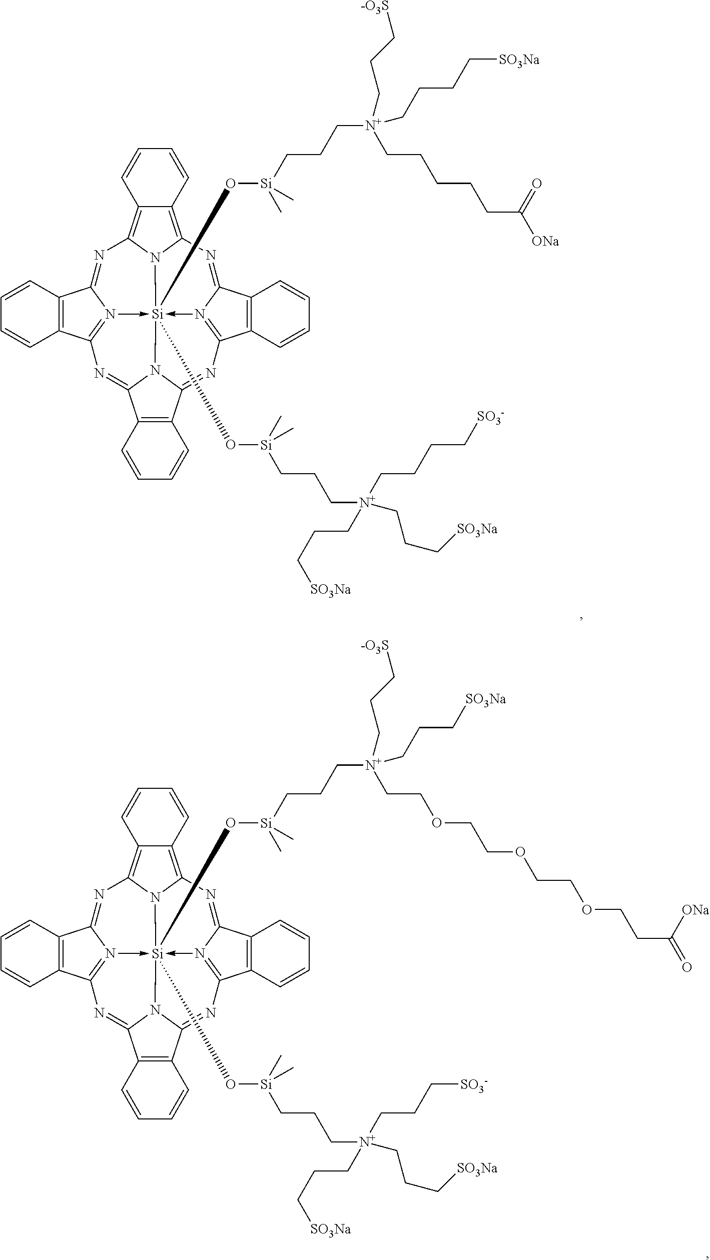

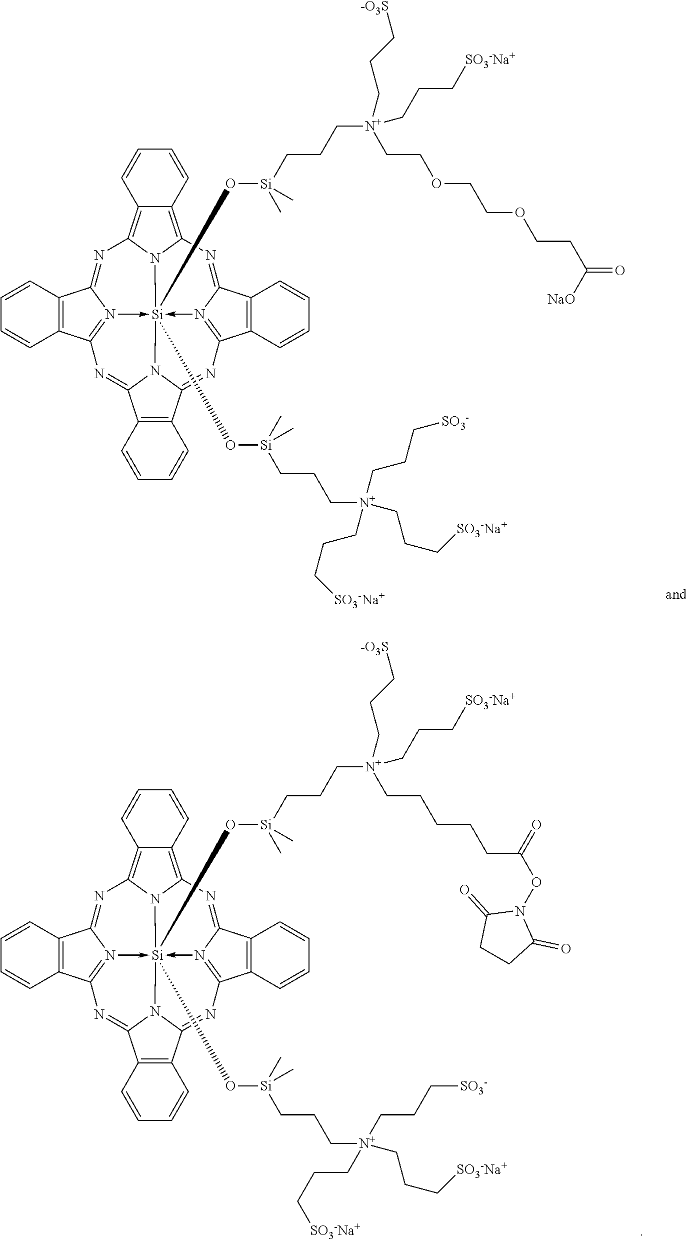

In some embodiments, the dye is a silicon phthalocyanine dye selected from the compounds provided in Table A, or a salt, ionic form, stereoisomer, or tautomer thereof:

TABLE A

| Dye | |
|---|---|
| Compound | Structure |
| No. | Name |
| 1 | 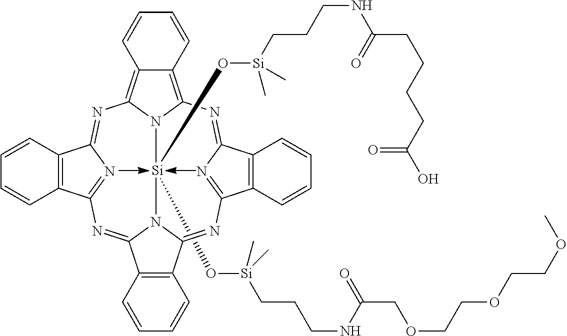 [SMILES: COCCOCCOCC(=O)NCCC[Si](C)(C)O[Si@@]1(O[Si](C)(C)CCCNC(=O)CCCCC(=O)O)n2c3c4ccccc4c2/N=C2N=C(/N=c4/c5ccccc5/c(n41)=N/C1=NC(=N\3)/c3ccccc31)c1ccccc1\2] |
| | 6-((3-(dimethyl((19-((15-methyl-10-oxo-2,5,8-trioxa-11-aza-15-silahexadecan-15-yl)- |
| | oxy)-19H-6,11-(azeno)-17,21-(azeno[3]episoindoloazeno)benzo[7,8][1,3,5,10]- |
| | tetraaza[2]silacycloundecino[4,3-a:11,1-a′]diisoindol-19-yl)oxy)silyl)propyl)amino)- |
| | 6-oxohexanoic acid |
| 5 | 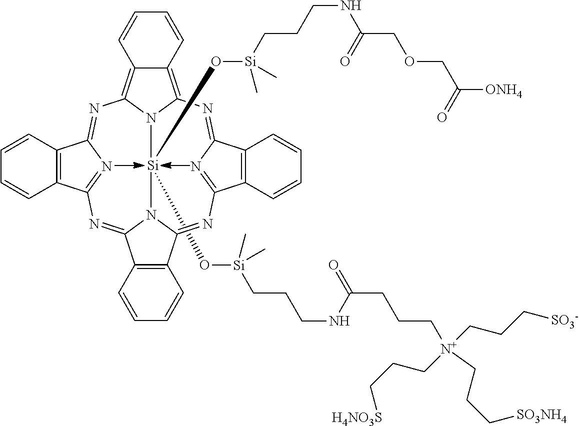 |
| | Ammonium 3-((4-((3-(((19-(((3-(2-(carboxymethoxy)acetamido)propyl)dimethylsilyl)oxy)- |
| | 19H-6,11-(azeno)-17,21-(azeno[3]episoindoloazeno)benzo[7,8][1,3,5,10]tetraaza[2]- |
| | silacycloundecino[4,3-a:11,1-a′]diisoindol-19-yl)oxy)dimethylsilyl)propyl)amino)-4- |
| | oxobutyl)bis(3-sulfopropyl)ammonio)propane-1-sulfonate |
| 12 | 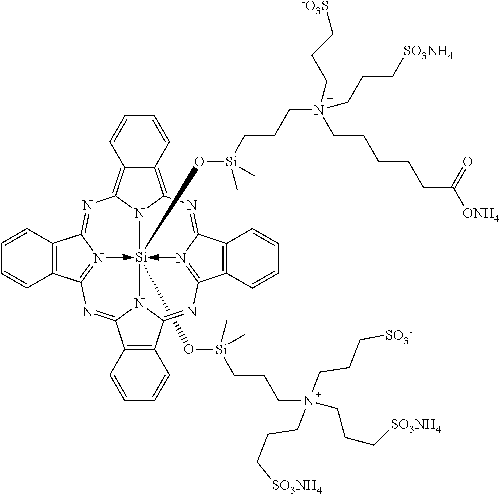 |
| | 3-((6-((25-azaneyl)oxy)-6-oxohexyl)(3-(((25-azaneyl)oxy)sulfonyl)propyl)(3- |
| | (((19-(((3-(bis(3-(((25-azaneyl)oxy)sulfonyl)propyl)(3-sulfonatopropyl)- |
| | ammonio)propyl)dimethylsilyl)oxy)-19H-6,11-(azeno)-17,21-(azeno[3]episo- |
| | indoloazeno)benzo[7,8][1,3,5,10]tetraaza[2]silacycloundecino[4,3-a:11,1-a′]- |
| | diisoindol-19-yl)oxy)dimethylsilyl)propyl)ammonio)propane-1-sulfonate |
| 13 | 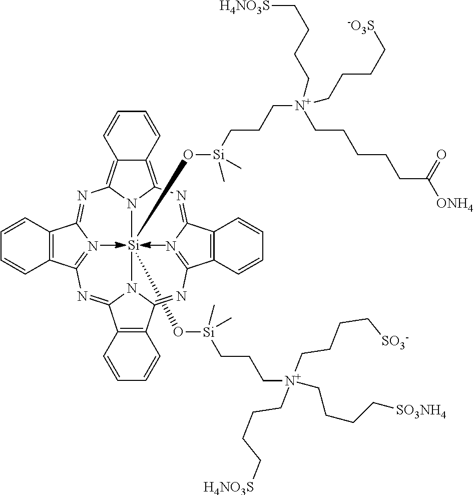 |
| | 4-((6-((25-azaneyl)oxy)-6-oxohexyl)(4-(((25-azaneyl)oxy)sulfonyl)butyl)(3- |
| | (((19-(((3-(bis(4-(((25-azaneyl)oxy)sulfonyl)butyl)(4-sulfonatobutyl)- |
| | ammonio)propyl)dimethylsilyl)oxy)-19H-6,11-(azeno)-17,21-(azeno[3]episo- |
| | indoloazeno)benzo[7,8][1,3,5,10]tetraaza[2]silacycloundecino[4,3-a:11,1-a′]- |
| | diisoindol-19-yl)oxy)dimethylsilyl)propyl)ammonio)butane-1-sulfonate |
| 14 | 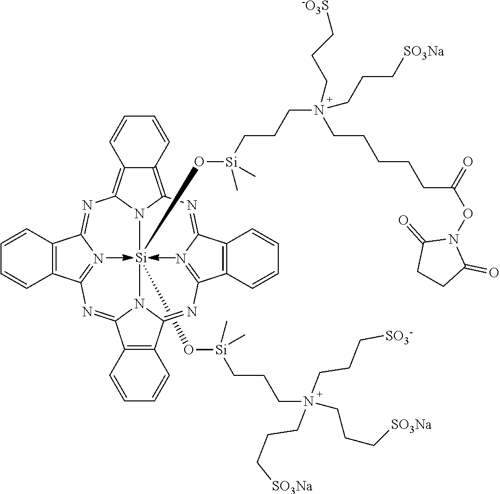 |
| | Sodium 3,3′,3″-((3-(((19-(((3-((6-((2,5-dioxopyrrolidin-1-yl)oxy)-6-oxohexyl)- |
| | bis(3-sulfonatopropyl)ammonio)propyl)dimethylsilyl)oxy)-19H-6,11-(azeno)- |
| | 17,21-(azeno[1]episoindoloazeno)benzo[7,8][1,3,5,10]tetraaza[2]silacyclo- |
| | undecino[4,3-a:11,1-a′]diisoindol-19-yl)oxy)dimethylsilyl)propyl)ammonio)- |
| | tris(propane-1-sulfonate) |
| 16 | 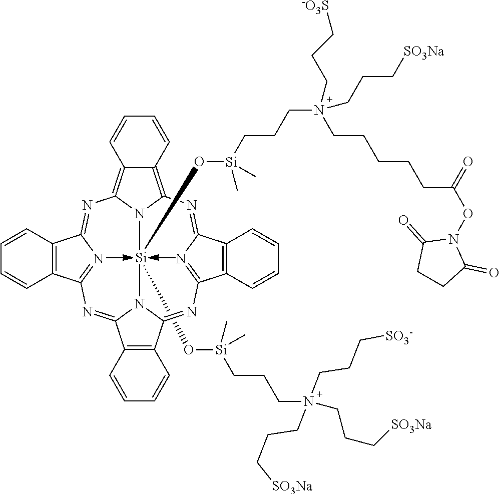 |
| | Sodium 4,4′,4″-((3-(((19-(((3-((6-((2,5-dioxopyrrolidin-1-yl)oxy)-6-oxohexyl)- |
| | bis(4-sulfonatobutyl)ammonio)propyl)dimethylsilyl)oxy)-19H-6,11-(azeno)- |
| | 17,21-(azeno[1]episoindoloazeno)benzo[7,8][1,3,5,10]tetraaza[2]silacyclo- |
| | undecino[4,3-a:11,1-a′]diisoindol-19-yl)oxy)dimethylsilyl)propyl)ammonio)- |
| | tris(butane-1-sulfonate) |
| 17 | 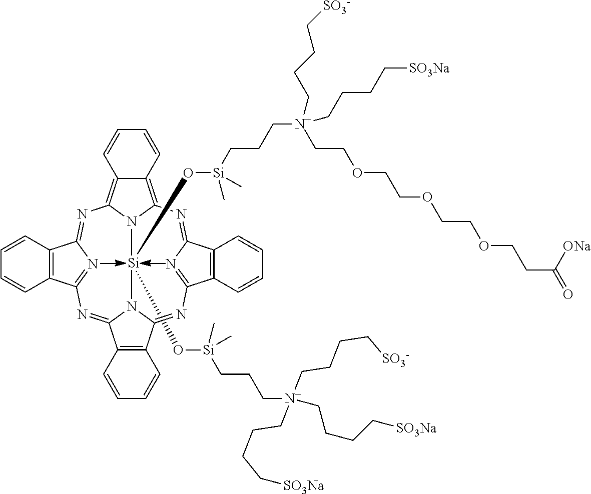 |
| | Sodium 2-((19-((dimethyl(3-(tris(4-sulfonatobutyl)ammonio)propyl)silyl)oxy)-19H-6,11- |
| | (azeno)-17,21-(azeno[3]episoindoloazeno)benzo[7,8][1,3,5,10]tetraaza[2]silacyclo- |
| | undecino[4,3-a:11,1-a′]diisoindol-19-yl)oxy)-2-methyl-6,6-bis(4-sulfonatobutyl)- |
| | 9,12,15-trioxa-6-aza-2-silaoctadecan-6-ium-18-oate |
| 18 | 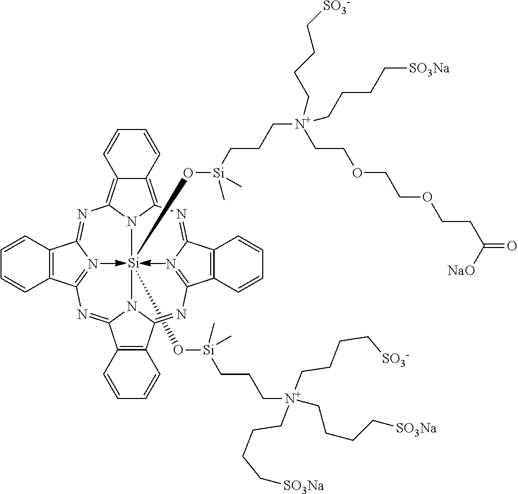 |
| | Sodium 2-((19-((dimethyl(3-(tris(4-sulfonatobutyl)ammonio)propyl)silyl)oxy)- |
| | 19H-6,11-(azeno)-17,21-(azeno[3]episoindoloazeno)benzo[7,8][1,3,5,10]tetraaza- |
| | [2]silacycloundecino[4,3-a:11,1-a′]diisoindol-19-yl)oxy)-2-methyl-6,6-bis(4- |
| | sulfonatobutyl)-9,12-dioxa-6-aza-2-silapentadecan-6-ium-15-oate |
| 19 | Sodium 2-((19-((dimethyl(3-(tris(3-sulfonatopropyl)ammonio)propyl)silyl)oxy)- |
| | 19H-6,11-(azeno)-17,21-(azeno[3]episoindoloazeno)benzo[7,8][1,3,5,10]tetraaza- |
| | [2]silacycloundecino[4,3-a:11,1-a′]diisoindol-19-yl)oxy)-2-methyl-6,6-bis(3- |
| | sulfonatopropyl)-9,12,15-trioxa-6-aza-2-silaoctadecan-6-ium-18-oate |

In particular embodiments, the phthalocyanine dye containing the reactive group has the structure of Formula (I):

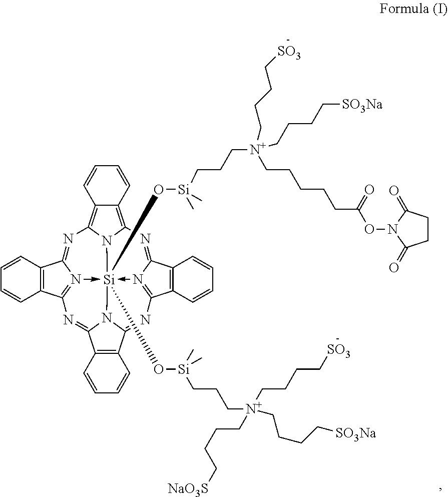

In particular embodiments, the phthalocyanine dye containing the reactive group has the structure of Formula (II):

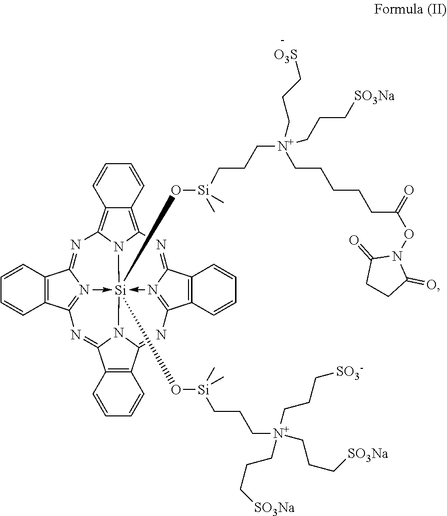

In some embodiments the compositions for use with the methods and uses provided herein include a conjugate comprising a Si-phthalocyanine dye linked to targeting molecule, wherein the targeting molecule binds to CD25. In some embodiments, the targeting molecule is an IL-2 non-blocking anti-CD25 antibody or an antigen-binding fragment thereof. In some embodiments, the composition comprises an IL-2 non-blocking anti-CD25-Si-phthalocyanine dye conjugate. In some embodiments, the composition comprises an IL-2 non-blocking anti-CD25-IR700 conjugate. In some embodiments, the composition comprises an IL-2 non-blocking anti-CD25-IR700 conjugate, where the anti-CD25 portion is 7G7B6, MA251, “clone A” or “clone B.” In some embodiments, the composition comprises an IL-2 non-blocking anti-CD25-IR700 conjugate, where the IL-2 non-blocking anti-CD25 portion contains a functional Fc region. In some embodiments, the composition is an IL-2 non-blocking anti-CD25-IR700 conjugate, where the IL-2 non-blocking anti-CD25 portion contains an Fc region, such as a full-length Fc region. In some embodiments, the composition is an IL-2 non-blocking anti-CD25-IR700 conjugate, where the IL-2 non-blocking anti-CD25 portion contains an Fc region, such as an Fc region that is engineered to exhibit antibody-dependent cellular cytotoxicity (ADCC) activity or exhibit enhanced ADCC activity.

### IV. CHECKPOINT INHIBITOR COMBINATION THERAPY

The methods and uses provided herein can include the administration of an immune checkpoint inhibitor prior to, concurrent with, or subsequent to the administration of the conjugate. For example, the methods can include administering one or more doses of an immune checkpoint inhibitor, administering a conjugate comprising a phthalocyanine dye, such as a Si-phthalocyanine dye, linked to a targeting molecule, wherein the targeting molecule binds to CD25 without blocking IL-2, and after administration of the conjugate, illuminating a first tumor and, optionally one or more additional tumors. The methods can include first administering a conjugate comprising a phthalocyanine dye linked to targeting molecule, wherein the targeting molecule binds to CD25 without blocking IL-2, and after administration of the conjugate, illuminating a first tumor and, optionally one or more additional tumors, and then administering an immune checkpoint inhibitor subsequent either to administration of the conjugate or subsequent to the illumination (e.g., irradiation) step. The methods can also include the administration of an immune checkpoint inhibitor concurrently with administration of the conjugate. In some aspects, the provided combinations can include a phthalocyanine dye-targeting molecule conjugate (e.g., IL-2 non-blocking anti-CD25 antibody-IR700 conjugate) and an immune checkpoint inhibitor. In some aspects, such combinations can be employed in the provided methods or uses, such as methods or uses related to a combination therapy or treatment.

In some embodiments, the immune checkpoint inhibitor is selected from a PD-1 inhibitor, a PD-L1 inhibitor, a CTLA-4 inhibitor, or a combination thereof. In some embodiments, the immune checkpoint inhibitor is selected from an antibody or antigen-binding fragment that binds to PD-1, an antibody or antigen-binding fragment that binds to PD-L1 or an antibody or antigen-binding fragment that binds to CTLA-4, or a combination thereof.

In some embodiments of the methods and uses provided herein, the immune checkpoint inhibitor is an anti-PD-1 antibody. In some embodiments, an anti-PD-1 antibody for use with the methods include, but are not limited to, pembrolizumab (MK-3475, KEYTRUDA; lambrolizumab), nivolumab (OPDIVO), cemiplimab (LIBTAYO), toripalimab (JS001), HX008, SG001, GLS-010, dostarlimab (TSR-042), tislelizumab (BGB-A317), cetrelimab (JNJ-63723283), pidilizumab (CT-011), genolimzumab (APL-501, GB226), BCD-100, cemiplimab (REGN2810), F520, sintilimab (IB1I308), CS1003, LZM009, camrelizumab (SHR-1210), SCT-I10A, MGA012, AK105, PF-06801591, AMP-224, AB122, AMG 404, BI 754091, HLX10, JTX-4014, AMP-514 (MEDI0680), Sym021, MGD019, MGD013, AK104, XmAb20717, R07121661, CX-188, spartalizumab, BCD-217, HX009, IB1308, PDR001, REGN2810, TSR-042 (ANB011), or an antigen-binding fragment thereof or any combination thereof.

In some embodiments of the methods and uses provided herein, the immune checkpoint inhibitor is an anti-PD-L1 antibody. Anti-PD-L1 antibodies that can be used in the methods and uses provided herein include, but are not limited to, atezolizumab (MPDL3280A, Tecentriq, RG7446), avelumab (BAVENCIO, MSB0010718C; M7824), durvalumab (MEDI4736, IMFINZI), LDP, NM-01, STI-3031 (IMC-001; STI-A1015), KN035, LY3300054, M7824 (MSB0011359C), BMS-936559, MSB2311, BCD-135, BGB-A333, CBT-502 (TQB-2450), cosibelimab (CK-301), CS1001 (WPB3155), FAZ053, MDX-1105, SHR-1316 (HTI-1088), TG-1501, ZKAB001 (STI-A1014), INBRX-105, MCLA-145, KN046, LY3415244, REGN3504, HLX20, or an antigen-binding fragment thereof or any combination thereof.

In some embodiments of the methods and uses provided herein, the immune checkpoint inhibitor is an anti-CTLA-4 antibody. Exemplary anti-CTLA-4 antibodies are ipilimumab (YERVOY), tremelimumab (ticilimumab, CP-675,206), AGEN1181, AGEN1884, ADU-1064, BCD-145, BCD-217, ADG116, AK104, ATOR-1015, BMS-986218, KN046, MGD019, MK-1308, REGN4659, XmAb20717, XmAb22841, or an antigen-binding fragment thereof or any combination thereof.

In some embodiments of the methods and uses provided herein, the immune checkpoint inhibitor is selected from an antibody or antigen-binding fragment that binds to PD-1, PD-L1, or CTLA-4, and the conjugate is an IL-2 non-blocking anti-CD25-IR700 conjugate. In some embodiments of the methods and uses provided herein, the immune checkpoint inhibitor is antibody or antigen-binding fragment that binds to PD-1 and the conjugate is an IL-2 non-blocking anti-CD25-IR700 conjugate, where the IL-2 non-blocking anti-CD25 portion of the conjugate is or is derived from 7G7B6. In some embodiments of the methods and uses provided herein, the immune checkpoint inhibitor is antibody or antigen-binding fragment that binds to PD-1 and the conjugate is an IL-2 non-blocking anti-CD25-IR700 conjugate, where the anti-CD25 portion of the conjugate is, is derived from, or completes with 7G7B6, and the antibody portion of the conjugate includes a functional Fc region. In some embodiments of the method, the conjugate is an IL-2 non-blocking anti-CD25-IR700 conjugate, where the IL-2 non-blocking anti-CD25 portion of the conjugate is, is derived from, or competes with 7G7B6, that includes a functional Fc region. In some embodiments of the method, the conjugate is an IL-2 non-blocking anti-CD25-IR700 conjugate, where the IL-2 non-blocking anti-CD25 portion of the conjugate is derived from, or competes with 7G7B6, that includes a functional Fc region, and the anti-PD-1 antibody is pembrolizumab (MK-3475, KEYTRUDA), nivolumab (OPDIVO), or cemiplimab (LIBTAYO) or an antigen-binding fragment thereof.

In some embodiments of the methods and uses provided herein, an immune checkpoint inhibitor can be administered to a subject having cancer prior to the administration of IL-2 non-blocking anti-CD25-Si phthalocyanine dye conjugate (e.g., an IL-2 non-blocking anti-CD25-IR700 conjugate), concurrently to the administration of IL-2 non-blocking anti-CD25-Si phthalocyanine dye conjugate, after the administration of IL-2 non-blocking anti-CD25-Si phthalocyanine dye conjugate or any combination thereof.

In some embodiments of the methods and uses provided herein, an immune checkpoint inhibitor is administered one time, two times, three times, four times, five times, six times, seven times, eight times, nine times, 10 times, or more than 10 times to a subject.

In some embodiments, an immune checkpoint inhibitor is administered to a subject once, twice three times, four times, five times or more than five times prior to the administration of the conjugate. In some embodiments, an immune checkpoint inhibitor is administered to a subject at or about 12 hours, 24 hours, 48 hours, 96 hours, one week, two weeks, three weeks or four weeks prior to the administration of the conjugate. In some embodiments, an immune checkpoint inhibitor is administered to a subject less than 1-4 weeks or less than 1-3 weeks or 1-2 weeks prior to administration of the conjugate.

In some embodiments, an immune checkpoint inhibitor is administered to a subject once, twice three times, four times, five times or more than five times after to the administration of the conjugate. In some embodiments, an immune checkpoint inhibitor is administered to a subject prior to administration of the conjugate and after administration of the conjugate.

In some embodiments of the methods and uses provided herein, an IL-2 non-blocking anti-CD25 conjugate is administered to a subject once or more than once. In some embodiments, the conjugate is administered one time, two times, three times, four times, five times, six times, seven times, eight times, nine times, 10 times, or more than 10 times to the subject. In some embodiments, the first tumor(s) is illuminated after each administration of the conjugate, such as within 24 hours±4 hours after each administration of the conjugate. In some embodiments, the conjugate is administered more than once if a residual lesion remains in the subject, such as a first tumor or one or more additional tumors, residual cells or masses from a primary tumor, invasive cancer cells or metastatic tumor cells. In some embodiments, the dosing of the conjugate is repeated if a residual lesion remains at a time that is more than 2 weeks, 3 weeks, 4 weeks, 1 month, 2 months, 3 months, 4, months, 5 months, 6 months, 1 year or more than 1 year after the prior administration of the conjugate.

In some embodiments of the methods and uses provided herein, an IL-2 non-blocking anti-CD25 conjugate, such as an IL-2 non-blocking anti-CD25-IR700 conjugate, is administered and the methods also include administration of an immune checkpoint inhibitor, wherein the dual administration of results in an enhanced effects, such as an additional, additive, or synergistic effect. Additional, additive, or synergistic effect refers to an effect that is more than the effect of either monotherapy alone (i.e. administration of only the conjugate, administration of only the conjugate followed by illumination, or administration of only the checkpoint inhibitor to a subject). For example, the administration of an IL-2 non-blocking anti-CD25-IR700 conjugate and administration of a checkpoint inhibitor, such as an anti-PD-1 antibody, produces an effect that is greater than if only the anti-CD25-IR700 conjugate or only the checkpoint inhibitor is administered, or if only the anti-CD25-IR700 conjugate is administered followed by illumination. In some aspects, the provided methods and uses results in an additional, additive, or synergistic anti-tumor response; for example, the growth and/or increase in volume, dimension(s), or mass of one or both of the first tumor and the number of cells in the secondary population in the subject is inhibited to a greater degree or extent as compared with administration of only the conjugate, only the conjugate followed by illumination, and/or only the anti-PD-1 antibody. In some aspects of the provided methods or uses, the growth and/or increase in volume, dimension(s), or mass of one or both of the first tumor and the volume, dimension(s), or cell number of the secondary population in the subject is inhibited to a greater degree as compared with administration of only the conjugate followed by illumination and as compared with administration of only the anti-PD-1 antibody. In some embodiments, the inhibition includes one or more of: a less than 20% increase in tumor volume, tumor dimensions or tumor mass; no change in tumor volume, dimension, or mass (i.e., halted tumor growth or progression); or a reduction in tumor volume, tumor dimensions or tumor mass, or a reduction in the number of tumor cells. In some embodiments, the inhibition includes one or more of: a less than 20% increase in the number of tumor cells. In some embodiments, the methods produce an enhanced response, such as a more complete response, a more durable response or longer lasting response.

In some embodiments, the methods produce an enhanced effect, such as an additional effect, an additive effect, or a synergistic effect on a first tumor including one or more of: inhibition, reduction or elimination of tumor growth, reduction in tumor volume, dimension(s), or mass, or an increase in numbers of subjects with a complete response, and any combinations thereof. In some embodiments, the methods produce an enhanced effect, such as an additional effect, an additive effect, or a synergistic effect, on invasive tumor cells including one or more of: inhibition, reduction or elimination of tumor cell growth, reduction in number or volume, dimension(s), or mass of invasive tumor cells, and any combinations thereof, including combinations with effects on one or more illuminated tumors, such as the first tumor. In some embodiments, the methods produce an enhanced effect, such as an additional effect, an additive effect, or a synergistic effect, on metastatic tumor cells including one or more of: inhibition, reduction or elimination of metastatic tumor cell growth, reduction in number or volume, dimension(s), or mass of metastatic tumor cells, and any combinations thereof, including combinations with effects on one or more illuminated tumors. In some embodiments, the synergistic effect is achieved on the tumor exposed directly to the illumination (i.e., illumination or irradiation with the selected wavelength of light). In some embodiments, the enhanced effect is achieved on tumor cells that were not illuminated, such as where a first tumor is illuminated, and a synergistic effect is achieved on metastatic tumor cells or invasive tumor cells that were not illuminated, such as metastatic tumor cells or invasive tumor cells located distal to the illuminated tumor that were not illuminated.

In some embodiments, the methods produce a synergistic effect on the increase or augmentation of systemic immunity. In some embodiments, the methods produce a synergistic effect on any of the measures of systemic immunity as described herein or known, such as those described in Section II herein, for example, the number of intratumoral CD8⁺ T lymphocytes, the ratio of CD8⁺ T lymphocytes to regulatory T cells (Tregs), intratumoral T lymphocyte exhaustion (e.g., the percentage of CD3+CD8⁺ cells that express PD-1 and/or CTLA4 markers), the number or percentage of intratumoral activated CD8⁺ T lymphocytes (e.g., Ki67⁺ or CD69⁺ CD8 cells as a percentage of CD45⁺ cells), the expansion of cytotoxic intratumoral T lymphocytes (e.g., the percentage of CD3+CD8⁺ cells that do not express PD-1 and/or CTLA4 markers), based on the splenocyte cytotoxicity against tumor cells, or any or all of combination thereof. In some aspects, intratumoral CD8⁺ T lymphocytes include CD3+CD8⁺ cells, intratumoral exhausted T lymphocytes include PD-1⁺CTLA-4⁺CD3⁺CD8⁺ cells, activated intratumoral CD8⁺ T lymphocytes include CD3⁺CD8⁺Ki67⁺ and/or CD3⁺CD8⁺CD69⁺ cells, expansion of cytotoxic T lymphocytes include PD-1I-CTLA-4⁻CD3⁺CD8⁺ cells.

In some embodiments, the methods include administration of an IL-2 non-blocking anti-CD25-IR700 conjugate and an immune checkpoint inhibitor to achieve a synergistic effect. In some embodiments, the methods include administration of an IL-2 non-blocking anti-CD25-IR700 conjugate and an anti-PD-1 antibody, to achieve an enhanced effect, such as a synergistic effect or an additive effect. In some embodiments, the anti-CD25-IR700 conjugate comprises basiliximab with a function Fc region and the immune checkpoint inhibitor is an anti-PD-1 antibody such as nivolumab, and an enhanced effect, such as an additional, additive, or synergistic effect, is seen as a reduction of tumor growth, reduction of tumor volume, reduction in tumor dimension, reduction in tumor mass, or a complete response, in an illuminated tumor, and/or a reduction in the growth, number, volume, dimension, or mass of non-illuminated tumor cells in a secondary population of tumor cells (such as invasive tumor cells or metastatic tumor cells). In some embodiments, administration of an IL-2 non-blocking anti-CD25-IR700 conjugate and illumination results in a reduction of the number of intratumoral Tregs and/or an increase of intratumoral CD8⁺ to Treg ratio or intratumoral CD4⁺ to Treg ratio. In some embodiments, a combination treatment with an anti-PD-1 antibody can result in a synergistic effect with respect to a reduction of the number of intratumoral Tregs and/or an increase of intratumoral CD8⁺ to Treg ratio or intratumoral CD4⁺ to Treg ratio.

### V. ADDITIONAL THERAPEUTIC AGENT

In some aspects, the provided methods and uses involve administration of an additional therapeutic agent or anti-cancer treatment. In some aspects, the additional therapeutic agent is an immune modulating agent. In some aspects, the additional therapeutic agent is an anti-cancer treatment.

In some embodiments, the immune-modulating agent is cytokine. In some embodiments, the immune modulating agent is a cytokine or is an agent that induces increased expression of a cytokine in the tumor microenvironment. In some aspects, “cytokine” refers to a generic term for proteins released by one cell population that act on another cell as intercellular mediators. Examples of such cytokines are lymphokines, monokines, and traditional polypeptide hormones. Included among the cytokines are growth hormones such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen; tumor necrosis factor-alpha and -beta; mullerian-inhibiting substance; mouse gonadotropin-associated peptide; inhibin; activin; vascular endothelial growth factor; integrin; thrombopoietin (TPO); nerve growth factors such as NGF-beta; platelet-growth factor; transforming growth factors (TGFs) such as TGF-alpha and TGF-beta; insulin-like growth factor-I and —II; erythropoietin (EPO); osteoinductive factors; interferons such as interferon-alpha, beta, and -gamma; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1 alpha, IL-2, IL-3, IL-4, IL-5, IL-6, IL-7, IL-8, IL-9, IL-10, IL-11, IL-12; IL-15, a tumor necrosis factor such as TNF-alpha or TNF-beta; and other polypeptide factors including LIF and kit ligand (KL). As used herein, the term cytokine includes proteins from natural sources or from recombinant cell culture, and biologically active equivalents of the native sequence cytokines. For example, the immune modulating agent is a cytokine and the cytokine is IL-4, TNF-α, GM-CSF or IL-2.

In some embodiments, the immune modulating agent is selected from among GM-CSF, CpG-ODN (CpG oligodeoxynucleotides), lipopolysaccharide (LPS), monophosphoryl lipid A (MPL), alum, recombinant Leishmania polyprotein, imiquimod, MF59, poly I:C, poly A:U, type 1 IFN, Pam3Cys, Pam2Cys, complete freund's adjuvant (CFA), alpha-galactosylceramide, RC-529, MDF2P, Loxoribine, anti-CD40 agonist, SIRPa antagonist, AS04, AS03, Flagellin, Resiquimod, DAP (diaminopimelic acid), MDP (muramyl dipeptide) and CAF01 (cationic adjuvant formulation-01). In some embodiments, the immune modulating agent is a Toll-like receptor (TLR) agonist, an adjuvant or a cytokine. In some embodiments, the immune modulating agent is a TLR agonist and the TLR agonist is TLR agonist is a TLR4 agonist, a TLR7 agonist, a TLR8 agonist, or a TLR9 agonist. In some embodiments, the TLR agonist is selected from among triacylated lipoprotein, diacylated lipopeptide, lipoteichoic acid, peptidoglycan, zymosan, Pam3CSK4, dsRNA, poly(I:C), Poly G10, Poly G3, CpG, 3M003, flagellin, lipopolysaccharide (LPS) Leishmania homolog of eukaryotic ribosomal elongation and initiation factor 4a (LeIF), MED 19197, SD-101, and imidazoquinoline TLR agonists.

In some embodiments, the immune modulating agent can contain one or more interleukins or other cytokines. For example, the interleukin can include leukocyte interleukin injection (Multikine), which is a combination of natural cytokines.

In some embodiments, the immune modulating agent is a Toll-like receptor (TLR) agonist. In some embodiments, such agonists can include a TLR4 agonist, a TLR8 agonist, or a TLR9 agonist. Such an agonist may be selected from peptidoglycan, poly(I:C), CpG, 3M003, flagellin, and Leishmania homolog of eukaryotic ribosomal elongation and initiation factor 4a (LeIF).

In some embodiments, the immune modulating agent can be one that enhances the immunogenicity of tumor cells such as patupilone (epothilone B), epidermal-growth factor receptor (EGFR)-targeting monoclonal antibody 7A7.27, histone deacetylase inhibitors (e.g., vorinostat, romidepsin, panobinostat, belinostat, and entinostat), the n3-polyunsaturated fatty acid docosahexaenoic acid, proteasome inhibitors (e.g., bortezomib), shikonin (the major constituent of the root of Lithospermum erythrorhizon) and oncolytic viruses, such as TVec (Talimogene laherparepvec). In some embodiments, the immune modulating agent activates immunogenic cell death of the cancer or tumor, such as anthracyclines (e.g., doxorubicin, mitoxantrone), BK channel agonists, bortezomib, bortezomib plus mitomycin C plus hTert-Ad, Cardiac glycosides plus non-ICD inducers, cyclophosphamide, GADD34/PP1 inhibitors plus mitomycin, LV-tSMAC, and oxaliplatin. In some embodiments, the immune modulating agent can be an epigenetic therapy, such as DNA methyltransferase inhibitors (e.g., Decitabine, 5-aza-2′-deoxycytidine).

For example, in some embodiments, the immune modulating agent can be a DNA methyltransferase inhibitor, which can regulate expression of tumor associated antigens (TAA). TAAs are antigenic substances produced in tumor cells which triggers an immune response. TAAs are often downregulated by DNA methylation in tumors to escape the immune system. Reversal of DNA methylation restores TAA expression, increasing the immunogenicity of tumor cells. For example, demethylating agents such as decitabine (5-aza-2′-deoxycytidine) can upregulate expression of TAAs in tumor cells and increase immune recognition of the cancerous cells. Photoimmunotherapy would further expose TAAs to the immune system by disrupting cells.

In some embodiments, the additional therapeutic agent is an agent or compound, used in anti-cancer treatment, such as anti-cancer agents. These include any agents, that can alleviate, reduce, ameliorate, prevent, or place or maintain in a state of remission of clinical symptoms or diagnostic markers associated with tumors and cancer, and can be used in combinations and compositions provided herein. In some embodiments, the anti-cancer agent is one whose therapeutic effect is generally associated with penetration or delivery of the anti-cancer agent into the tumor microenvironment or tumor space. In some embodiments, the anti-cancer agent is an alkylating agent, a platinum drug, an antimetabolite, an anti-tumor antibiotic, a topoisomerase inhibitor, a mitotic inhibitor, a corticosteroid, a proteasome inhibitor, a kinase inhibitor, a histone-deacetylase inhibitor or an antibody or antigen-binding antibody fragment thereof. In some embodiments, the anti-cancer agent is a peptide, protein or small molecule drug.

In some embodiments, the anti-cancer agent is 5-Fluorouracil/leucovorin, oxaliplatin, irinotecan, regorafenib, ziv-aflibercept, capecitabine, cisplatin, paclitaxel, topotecan, carboplatin, gemcitabine, docetaxel, 5-FU, ifosfamide, mitomycin, pemetrexed, vinorelbine, carmustine wager, temozolomide, methotrexate, capecitabine, lapatinib, etoposide, dabrafenib, vemurafenib, liposomal cytarabine, cytarabine, interferon alpha, erlotinib, vincristine, cyclophosphamide, lomustine, procarbazine, sunitinib, somatostatin, doxorubicin, pegylated liposomal encapsulated doxorubicin, epirubicin, eribulin, albumin-bound paclitaxel, ixabepilone, cotrimoxazole, taxane, vinblastine, temsirolimus, temozolomide, bendamustine, oral etoposide, everolimus, octreotide, lanreotide, dacarbazine, mesna, pazopanib, eribulin, imatinib, regorafenib, sorafenib, nilotinib, dasantinib, celecoxib, tamoxifen, toremifene, dactinomycin, sirolimus, crizotinib, certinib, enzalutamide, abiraterone acetate, mitoxantrone, cabazitaxel, fluoropyrimidine, oxaliplatin, leucovorin, afatinib, ceritinib, gefitinib, cabozantinib, oxaliplatin or aurora pyrimidine.

In some embodiments, the anti-cancer agent is an alkylating agent. Alkylating agents are compounds that directly damage DNA by forming covalent bonds with nucleic acids and inhibiting DNA synthesis. Exemplary alkylating agents include, but are not limited to, mechlorethamine, cyclophosphamide, ifosfamide, melphalan, chlorambucil, busulfan, and thiotepa as well as nitrosourea alkylating agents such as carmustine and lomustine.

In some embodiments, the anti-cancer agent is an antibody or antigen-binding antibody fragment.

In some embodiments, the anti-cancer agent is a platinum drug. Platinum drugs bind to and cause crosslinking of DNA, which ultimately triggers apoptosis. Exemplary platinum drugs include, but are not limited to, cisplatin, carboplatin, oxaliplatin, satraplatin, picoplatin, nedaplatin, triplatin, and lipoplatin.

In some embodiments, the anti-cancer agent is an antimetabolite. Antimetabolites interfere with DNA and RNA growth by substituting for the normal building blocks of RNA and DNA. These agents damage cells during the S phase, when the cell's chromosomes are being copied. In some cases, antimetabolites can be used to treat leukemias, cancers of the breast, ovary, and the intestinal tract, as well as other types of cancer. Exemplary antimetabolites include, but are not limited to, 5-fluorouracil (5-FU), 6-mercaptopurine (6-MP), capecitabine (Xeloda®), cytarabine (Ara-C®), floxuridine, fludarabine, gemcitabine (Gemzar®), hydroxyurea, methotrexate, and pemetrexed (Alimta®).

In some embodiments, the anti-cancer agent is an anti-tumor antibiotic. Anti-tumor antibiotics work by altering the DNA inside cancer cells to keep them from growing and multiplying. Anthracyclines are anti-tumor antibiotics that interfere with enzymes involved in DNA replication. These drugs generally work in all phases of the cell cycle. They can be widely used for a variety of cancers. Exemplary anthracyclines include, but are not limited to, daunorubicin, doxorubicin, epirubicin, and idarubicin. Other anti-tumor antibiotics include actinomycin-D, bleomycin, mitomycin-C, and mitoxantrone.

In some embodiments, the anti-cancer agent is a topoisomerase inhibitor. These drugs interfere with enzymes called topoisomerases, which help separate the strands of DNA so they can be copied during the S phase. Topoisomerase inhibitors can be used to treat certain leukemias, as well as lung, ovarian, gastrointestinal, and other cancers. Exemplary topoisomerase inhibitors include, but are not limited to, doxorubicin, topotecan, irinotecan (CPT-11), etoposide (VP-16), teniposide, and mitoxantrone.

In some embodiments, the anti-cancer agent is a mitotic inhibitor. Mitotic inhibitors are often plant alkaloids and other compounds derived from natural plant products. They work by stopping mitosis in the M phase of the cell cycle but, in some cases, can damage cells in all phases by keeping enzymes from making proteins needed for cell reproduction. Exemplary mitotic inhibitors include, but are not limited to, paclitaxel (Taxol®), docetaxel (Taxotere®), ixabepilone (Ixempra®), vinblastine (Velban®), vincristine (Oncovin®), vinorelbine (Navelbine®), and estramustine (Emcyt®).

In some embodiments, the anti-cancer agent is a corticosteroid. Corticosteroids, often simply called steroids, are natural hormones and hormone-like drugs that are useful in the treatment of many types of cancer. Corticosteroids can also be used before chemotherapy to help prevent allergic reactions as well as during and after chemotherapy to help prevent nausea and vomiting. Exemplary corticosteroids include, but are not limited to, prednisone, methylprednisolone (Solumedrol®), and dexamethasone (Decadron®).

In some embodiments, the anti-cancer agent is another type of chemotherapy drug, such as a proteasome inhibitor, a kinase inhibitor, or a histone-deacetylase inhibitor. In other embodiments, the anti-cancer agent is a biologic such as an antibody used in cancer therapy.

### VI. ILLUMINATION AND DEVICES FOR USE WITH THE METHODS AND COMPOSITIONS

In some aspects, devices that can be used with the provided embodiments include light diffusing devices that provide illumination (in some cases, also referred to as irradiation) at a wavelength (or wavelengths) of light wavelength suitable for use with the dye conjugate composition, such as a phthalocyanine dye conjugate (e.g., an IL-2 non-blocking anti-CD25-IR700 conjugate such as those described herein). Illumination devices can include a light source (for example, a laser), and a means of conveying the light to the area of interest (for example, one or more fibers to illuminate an isolated area of a subject or an isolated lesion or tumor). Exemplary illumination devices are described in U.S. Pat. Nos. 10,295,719; 10,527,771; and 10,416,366, incorporated herein by reference. Such devices deliver light to a target region of a subject using a light diffusing device, containing, a non-circular core optic fiber that is operably connected to a laser. In some embodiments, the core optic fiber is circular and is coiled or bent prior to interfacing with a light diffusing device. In particular aspects, the device delivers a “top hat” core irradiance distribution to deliver uniform light to the illuminated area. The light diffusing device can be as cylindrical diffuser for use, for example, for intratumor or intratissue irradiation. In some embodiments, the light diffusing device is a frontal diffuser, with a lens, where the illumination is projected through the lens of the frontal diffuser at the end of the optic fiber. The projected light can be a collimated or dispersing beam of light.

In some embodiments, the target area, such as a tumor, the vicinity of a tumor, a lymph node, the vicinity of the lymph node, is illuminated with light at a wavelength within a range from at or about 400 nm to at or about 900 nm, such as from or from at or about 500 nm to at or about 900 nm, such as from or from at or about 600 nm to at or about 850 nm, such as from or from at or about 600 nm to at or about 740 nm, such as from at or about 660 nm to at or about 740 nm, from at or about 660 nm to at or about 710 nm, from at or about 660 nm to at or about 700 nm, from at or about 660 to at or about 685, from at or about 665 to at or about 680, from at or about 670 to at or about 685, from at or about 670 nm to at or about 690 nm, from at or about 670 to at or about 680, from at or about 680 nm to at or about 740 nm, or from at or about 690 nm to at or about 710 nm. In some embodiments, the target area, such as a tumor, the vicinity of a tumor, a lymph node, the vicinity of the lymph node, or the tumor microenvironment, is illuminated with light at a wavelength of at or about 600 nm to at or about 850 nm, such as at or about 660 nm to at or about 740 nm. In some embodiments, the target area, such as a tumor, the vicinity of a tumor, a lymph node, the vicinity of the lymph node, or the tumor microenvironment, is illuminated with light at wavelength of at least at or about 600 nm, 620 nm, 640 nm, 660 nm, 680, nm, 700 nm, 720 nm or 740 nm, such as at or about 690±50 nm, or at or about 690±40 nm, for example, at or about 690 nm or at or about 680 nm. In some embodiments, the target area, such as a tumor, the vicinity of a tumor, a lymph node, the vicinity of the lymph node, or the tumor microenvironment, is illuminated with light at wavelength that is at or about 670±50 nm, or at or about 670±40 nm, for example, at or about 670 nm. In some embodiments, the target area, such as a tumor, the vicinity of a tumor, a lymph node, the vicinity of the lymph node, or the tumor microenvironment, is illuminated with light at a wavelength of less than or less than about 685 nm or 680 nm.

In some of any embodiments, the wavelength of light used for illumination depends on the phthalocyanine dye. For instance, if the Si-phthalocyanine dye is IR700, the illuminating step can be performed at a wavelength of 690 nm±20 nm. In some instance, if the Si-phthalocyanine dye comprises Formula (I)

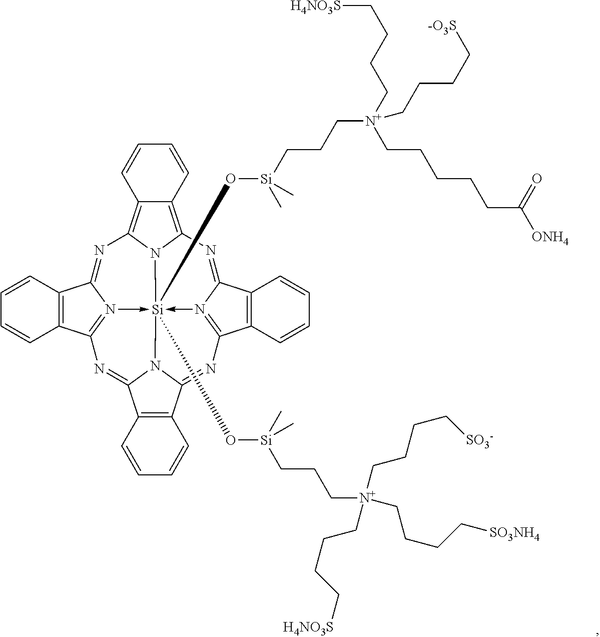

or a salt, stereoisomer, or tautomer thereof, the illuminating step can be performed at a wavelength of 660 nm±50 nm.

In some embodiments of the methods and uses provided herein, interstitial illumination is carried out using cylindrical diffusing fibers that includes a diffuser length of at or about 0.5 cm to at or about 10 cm and spaced at or about 1.8±0.2 cm apart. In some embodiments, the light (illumination) dose is from at or about 2 J/cm fiber length to at or about 500 J/cm fiber length. In some embodiments, the interstitial light (illumination) dose is from at or about 20 J/cm fiber length to at or about 500 J/cm fiber length. In some embodiments, the optical power (or optical fluence) of the interstitial light dose is at or about 100 mW/cm fiber length to at or about 500 mW/cm fiber length. In some embodiments, the light is administered for at or about 120 seconds to at or about 600 seconds. In some embodiments, the light is administered for at least at or about 100 seconds, 120 seconds, 150 seconds, 180 seconds, 200 seconds, 220 seconds, 250 seconds, 270 seconds, 300 seconds, 310 seconds, 330 seconds, 340 seconds, 350 seconds, 370 seconds, 380 seconds, 400 seconds, 420 seconds, 440 seconds, 460 seconds, 480 seconds, or 500 seconds. In some embodiments, the tumor is greater than at or about 10 mm deep or is a subcutaneous tumor.

In some embodiments, the provided methods include interstitial illumination of a target area that is a tumor, such as an interstitial tumor, in a subject with cylindrical diffusing fibers that includes a diffuser length of at or about 0.5 cm to at or about 10 cm and spaced at or about 1.8±0.2 cm apart with a light dose of at or about 100 J/cm fiber length with an optical fluence of at or about 400 mW/cm for at or about 250 seconds. In some embodiments, the target area is a tumor that is greater than at or about 10 mm deep or is a subcutaneous tumor. In some embodiments, the cylindrical diffusing fibers are placed in a catheter positioned in the tumor at or about 1.8±0.2 cm apart. In some embodiments, the catheter is optically transparent.

In some embodiments, the target area, such as a tumor, the vicinity of a tumor, a lymph node, the vicinity of the lymph node, or the tumor microenvironment, is illuminated with light dose, such as a superficial light dose, of at least at or about 1 J/cm², such as at least at or about 10 J/cm², at least at or about 30 J/cm², at least at or about 50 J/cm², at least at or about 100 J/cm², or at least at or about 500 J/cm². In some embodiments, the dose of illumination is from at or about 1 to at or about J/cm², from at or about 1 to at or about 500 J/cm², from at or about 5 to at or about 200 J/cm², from at or about 10 to at or about 100 J/cm², from at or about 10 to at or about 50 J/cm², or from at or about 25 to at or about 400 J/cm². In some embodiments, the target area is illuminated at a dose of at least at or about 2 J/cm², 5 J/cm², 10 J/cm², 25 J/cm², 30 J/cm², 50 J/cm², 75 J/cm², 100 J/cm², 150 J/cm², 200 J/cm², 300 J/cm², 400 J/cm², or 500 J/cm². In some embodiments, the light (illumination) dose is from at or about 25 J/cm² to at or about 400 J/cm². In some embodiments, the optical power (or optical fluence) of the light dose is at or about 50 mW/cm² to at or about 200 mW/cm², such as an optical power of at or about 50, 60, 70, 80, 90, 100, 110, 120, 130, 140, 150, 160, 170, 180, 190, or 200 mW/cm². In some embodiments, the light is administered for at or about 120 seconds to at or about 600 seconds. In some embodiments, the light is administered for at least at or about 100 seconds, 120 seconds, 150 seconds, 180 seconds, 200 seconds, 220 seconds, 250 seconds, 270 seconds, 300 seconds, 310 seconds, 330 seconds, 340 seconds, 350 seconds, 370 seconds, 380 seconds, 400 seconds, 420 seconds, 440 seconds, 460 seconds, 480 seconds, or 500 seconds.

In some embodiments, the target area is a tumor that is a superficial tumor. In some embodiments, the tumor is less than 10 mm thick. In some embodiments, illumination is carried out using a microlens-tipped fiber for surface or superficial illumination. In some embodiments, the light illumination dose is from at or about 5 J/cm² to at or about 200 J/cm². In some embodiments, the light illumination dose is from at or about 25 J/cm² to at or about 400 J/cm². In some embodiments, the optical power of the light dose is at or about 50 mW/cm² to at or about 200 mW/cm². In some embodiments, the light illumination dose is at or about 50 J/cm² at an optical power of at or about 150 mW/cm² for at or about 333 seconds.

In some embodiments, the target area, such as a tumor, the vicinity of a tumor, a lymph node, the vicinity of the lymph node, or the tumor microenvironment, are illuminated at a dose of at least at or about 1 J/cm fiber length, such as at least at or about 10 J/cm fiber length, at least at or about 50 J/cm fiber length, at least at or about 100 J/cm fiber length, at least at or about 250 J/cm fiber length, or at least at or about 500 J/cm fiber length. In some embodiments, the dose of illumination is from at or about 1 to at or about 1000 J/cm fiber length, from at or about 1 to at or about 500 J/cm fiber length, from at or about 2 to at or about 500 J/cm fiber length, from at or about 50 to at or about 300 J/cm fiber length, from at or about 10 to at or about 100 J/cm fiber length, or from at or about 10 to at or about 50 J/cm fiber length. In some embodiments, the target area, such as a tumor, the vicinity of a tumor, a lymph node, the vicinity of the lymph node, or the tumor microenvironment, is illuminated at a dose of at least at or about 2 J/cm fiber length, 5 J/cm fiber length, 10 J/cm fiber length, 25 J/cm fiber length, 50 J/cm fiber length, 75 J/cm fiber length, 100 J/cm fiber length, 150 J/cm fiber length, 200 J/cm fiber length, 250 J/cm fiber length, 300 J/cm fiber length, 400 J/cm fiber length or 500 J/cm fiber length. In some embodiments, the illumination is administered at a dose of at or about 100 J/cm with a fluence rate of at or about 400 mW/cm for at or about 250 seconds.

In some embodiments, the provided methods include illuminating a target area that is a superficial tumor in a subject with a microlens-tipped fiber for surface illumination with a light dose of from at or about 5 J/cm² to at or about 200 J/cm². In some embodiments, the light illumination dose is from at or about 25 J/cm² to at or about 400 J/cm². In some embodiments, the light illumination dose is at or about 50 J/cm². In some embodiments, the illumination of the superficial tumor is administered at a dose of at or about 50 J/cm² with a fluence rate of at or about 150 mW/cm² for at or about 333 seconds.

In some cases, it is found that a dose of illumination in a human subject to achieve PIT can be less than is necessary for PIT in a mouse. For example, in some cases, at or about 50 J/cm² (50 J/cm²) light dosimetry in an in vivo tumor mouse model is not effective for PIT, which is in contrast to what can be observed in the clinic with human patients.

In some embodiments, the dose of illumination following administration of the composition comprising the phthalocyanine dye-targeting molecule conjugate is at least at or about 1 J/cm² or at least at or about 1 J/cm of fiber length at a wavelength of at or about 660-740 nm, for example, at least at or about 10 J/cm² or at least at or about 10 J/cm of fiber length at a wavelength of at or about 660-740 nm, at least at or about 50 J/cm² or at least at or about 50 J/cm of fiber length at a wavelength of at or about 660-740 nm, or at least at or about 100 J/cm² or at least at or about 100 J/cm of fiber length at a wavelength of at or about 660-740 nm. In some embodiments, the wavelength is 660-710 nm. In some embodiments, the dose of illumination following administration of the composition comprising the phthalocyanine dye-targeting molecule conjugate is at least at or about 1.0 J/cm² or at least at or about 1 J/cm of fiber length, at a wavelength of at or about 690 nm, for example, at least at or about 10 J/cm² or at least at or about 10 J/cm of fiber length, at a wavelength of at or about 690 nm, at least at or about 50 J/cm² or at least at or about 50 J/cm of fiber length, at a wavelength of at or about 690 nm, or at least at or about 100 J/cm² or at least at or about 100 J/cm of fiber length, at a wavelength of at or about 690 nm, for example, 1.0 to 500 J/cm² or 1.0 to 500 J/cm of fiber length, at a wavelength of at or about 690 nm. Exemplary illumination after administration of the conjugates or compositions provided herein include illuminating the target area at a wavelength of at or about 660 nm to at or about 740 nm at a dose of at least at or about 1 J/cm² or at least at or about 1 J/cm of fiber length.

In some embodiments, the dose of illumination following administration of the composition comprising the phthalocyanine dye-targeting molecule conjugate is at least at or about 1 J/cm² or at least at or about 1 J/cm of fiber length at a wavelength of at or about 600-800 nm, for example, at least at or about 1 J/cm² or at least at or about 1 J/cm of fiber length at a wavelength of at or about 620-720 nm, at least at or about 10 J/cm² or at least at or about 10 J/cm of fiber length at a wavelength of at or about 620-720 nm, at least at or about 50 J/cm² or at least at or about 50 J/cm of fiber length at a wavelength of at or about 620-720 nm, or at least at or about 100 J/cm² or at least at or about 100 J/cm of fiber length at a wavelength of at or about 620-720 nm. In some embodiments, the wavelength is 640-700 nm. In some embodiments, the dose of illumination following administration of the composition comprising the phthalocyanine dye-targeting molecule conjugate is at least at or about 1.0 J/cm² or at least at or about 1 J/cm of fiber length, at a wavelength of at or about 670 nm, for example, at least at or about 10 J/cm² or at least at or about 10 J/cm of fiber length, at a wavelength of at or about 670 nm, at least at or about 50 J/cm² or at least at or about 50 J/cm of fiber length, at a wavelength of at or about 670 nm, or at least at or about 100 J/cm² or at least at or about 100 J/cm of fiber length, at a wavelength of at or about 670 nm, for example, 1.0 to 500 J/cm² or 1.0 to 500 J/cm of fiber length, at a wavelength of at or about 670 nm. Exemplary illumination after administration of the conjugates or compositions provided herein include illuminating the target area at a wavelength of at or about 620 nm to at or about 720 nm at a dose of at least at or about 1 J/cm² or at least at or about 1 J/cm of fiber length.

In some embodiments, illuminating is carried out at a wavelength of at or about 600 nm to at or about 850 nm and at a dose of from at or about 25 J/cm² to at or about 400 J/cm² or from at or about 2 J/cm fiber length to at or about 500 J/cm fiber length. In some embodiments, the target area is illuminated at a wavelength of 690±40 nm. In some embodiments, target area is illuminated at a dose of at or about of 50 J/cm² or at or about 100 J/cm of fiber length.

In some embodiments, a light or laser may be applied to the dye molecules, such as cells containing the conjugate, for from at or about 5 seconds to at or about 5 minutes. For example, in some embodiments, the light or laser is applied for at or about 5, 10, 15, 20, 25, 30, 35, 40, 45 50 or 55 seconds, or for within a range between any of two such values, to activate the dye molecules. In some embodiments, the light or laser is applied for at or about 1, 1.5, 2, 2.5, 3, 3.5, 4, 4.5 or 5 minutes, or more, or within a range between any two of such values. In some embodiments, the length of time a light or laser is applied can vary depending, for example, on the energy, such as wattage, of the light or laser. For example, lights or lasers with a lower wattage may be applied for a longer period of time in order to activate the dye molecule.

In some embodiments, a light or laser may be applied for at or about 30 minutes to at or about 96 hours after administering the conjugate. For example, in some embodiments, the light or laser is applied at or at about 30, 35, 40, 45, 50 or 55 minutes after administering the conjugate, or within a range between any two of such values. In some embodiments, the light or laser is applied at or at about 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 hours after administering the conjugate, or is administered within a range between about any two of such values, such as, for example, between at or about 20 hours to at or about 28 hours, or about 24 hours±4 hours. In some embodiments, the light or laser is applied between or between about 1 and 24 hours, such as between at or about 1 and at or about 12 hours, at or about 12 and at or about 24 hours, at or about 6 and at or about 12 hours, or may be administered more than at or about 24 hours following administration of the conjugate. In some embodiments, the light or laser is applied at or about 36, 48, 72 or 96 hours after administering the conjugate. In some embodiments, the light or laser is applied at or at about 24 hours±4 hours after administering the conjugate.

In some embodiments, the target area, such as a tumor, the vicinity of a tumor, a lymph node, the vicinity of the lymph node, or the tumor microenvironment, or subjects, can be illuminated one or more times. Thus, illumination can be completed in a single day, or can be done repeatedly on multiple days with the same or a different dosage, such as illumination at least at or about 2 different times, 3 different times, 4 different times 5 different times or 10 different times. In some embodiments, repeated illuminations may be done on the same day, on successive days, or every 1-3 days, every 3-7 days, every 1-2 weeks, every 2-4 weeks, every 1-2 months, or at even longer intervals. In some embodiments, multiple illuminations are performed, such as at least 2, at least 3, or at least 4 illuminations, such as 2, 3, 4, 5, 6, 7, 8, 9 or 10 separate administrations.

In some embodiments, the dose or method of illumination differs depending on the type or morphology of the target area, such as a tumor, the vicinity of a tumor, a lymph node, the vicinity of the lymph node.

In some embodiments, the illumination employs a device with “top hat” irradiance distribution profile, such as those described in WO2018/080952 and US20180239074.

### VII. DEFINITIONS

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

As used herein, the singular forms “a,” “an,” and “the” include plural referents unless the context clearly dictates otherwise. For example, “a” or “an” means “at least one” or “one or more.” It is understood that aspects and variations described herein include “consisting” and/or “consisting essentially of” aspects and variations.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

The term “about” as used herein refers to the usual error range for the respective value readily known to the skilled person in this technical field. Reference to “about” a value or parameter herein includes (and describes) embodiments that are directed to that value or parameter per se. For example, description referring to “about X” includes description of “X”.

As used herein, a “conjugate” refers to a targeting molecule linked directly or indirectly to a photoactivatable dye, such as those produced by chemical conjugates and those produced by any other methods. For example, a conjugate can refer to a phthalocyanine dye, such as an IR700 molecule, linked directly or indirectly to one or more targeting molecules, such as to a polypeptide binds to or targets to a cell surface protein. A targeting molecule can be a polypeptide, more than one polypeptide, an antibody or a chemical moiety.

As used herein an “IL-2 non-blocking anti-CD25 conjugate” refers to a conjugate having a targeting molecule that binds to CD25 but does not substantially or significantly block binding of IL-2 to CD25 and/or does not substantially or significantly block or interfere with IL-2 signaling. An IL-2 non-blocking anti-CD25 conjugate can have a targeting molecule that is an antibody, antigen-binding fragment or other moiety that binds to CD25 but does not substantially or significantly block binding of IL-2 to CD25.

A “monoclonal antibody” is an antibody produced by a single clone of B lymphocytes or by a cell into which the light and heavy chain genes of a single antibody have been transfected. Monoclonal antibodies are produced by methods known to those of skill in the art, for instance by making hybrid antibody-forming cells from a fusion of myeloma cells with immune spleen cells. Monoclonal antibodies include humanized monoclonal antibodies.

“Specifically binds” refers to the ability of individual antibodies to specifically immunoreact with an antigen, such as a tumor-specific antigen, relative to binding to unrelated proteins, such as non-tumor proteins, for example, β-actin. For example, a CD25-specific binding agent binds substantially only the CD25 protein in vitro or in vivo. As used herein, the term “tumor-specific binding agent” includes tumor-specific antibodies and other agents that bind substantially only to a tumor-specific protein in that preparation.

“Antibody-IR700 molecule” or “antibody-IR700 conjugate” refers to a molecule that includes both an antibody, such as a tumor-specific antibody, conjugated to IR700. In some examples the antibody is a humanized antibody (such as a humanized monoclonal antibody) that specifically binds to a surface protein on a cancer cell.

“Antigen” refers to a compound, composition, or substance that can stimulate the production of antibodies or a T cell response in an animal, including compositions (such as one that includes a tumor-specific protein) that are injected or absorbed into an animal. An antigen reacts with the products of specific humoral or cellular immunity, including those induced by heterologous antigens, such as the disclosed antigens. “Epitope” or “antigenic determinant” refers to the region of an antigen to which B and/or T cells respond. In one embodiment, T cells respond to the epitope, when the epitope is presented in conjunction with an MHC molecule. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5, about 9, or about 8-10 amino acids in a unique spatial conformation. Methods of determining spatial conformation of epitopes include, for example, x-ray crystallography and nuclear magnetic resonance.

Examples of antigens include, but are not limited to, peptides, lipids, polysaccharides, and nucleic acids containing antigenic determinants, such as those recognized by an immune cell. In some examples, an antigen includes a tumor-specific peptide (such as one found on the surface of a cancer cell) or immunogenic fragment thereof.

“Immune checkpoint inhibitor” refers to a type of drug that blocks certain proteins made by some types of immune system cells, such as T cells, and some cancer cells. These proteins help keep immune responses in check and can keep T cells from killing cancer cells. When these proteins are blocked, the “brakes” on the immune system are released and T cells are able to kill cancer cells better. Examples of checkpoint proteins found on T cells or cancer cells include PD-1/PD-L1 and CTLA-4/B7-1/B7-2. Some immune checkpoint inhibitors are used to treat cancer.

As used herein, a combination refers to any association between or among two or more items. The combination can be two or more separate items, such as two compositions or two collections, can be a mixture thereof, such as a single mixture of the two or more items, or any variation thereof. The elements of a combination are generally functionally associated or related.

As used herein, “combination therapy” refers to a treatment in which a subject is given two or more therapeutic agents, such as at least two or at least three therapeutic agents, for treating a single disease. In some embodiments, each therapy can result in an independent pharmaceutical effect, and together can result in an additive or synergistic pharmaceutical effect.

As used herein, “treating” a subject with a disease or condition means that the subject's symptoms are partially or totally alleviated or remain static following treatment. Hence treating encompasses prophylaxis, therapy and/or cure. Prophylaxis refers to prevention of a potential disease and/or a prevention of worsening of symptoms or progression of a disease.

As used herein, “treatment” means any manner in which the symptoms of a condition, disorder or disease or other indication, are ameliorated or otherwise beneficially altered.

As used herein, “therapeutic effect” means an effect resulting from treatment of a subject that alters, typically improves or ameliorates the symptoms of a disease or condition or that cures a disease or condition.

As used herein, a “therapeutically effective amount” or a “therapeutically effective dose” refers to the quantity of an agent, compound, material, or composition containing a compound that is at least sufficient to produce a therapeutic effect. Hence, it is the quantity necessary for preventing, curing, ameliorating, arresting or partially arresting a symptom of a disease or disorder.

As used herein, amelioration of the symptoms of a particular disease or disorder by a treatment, such as by administration of a pharmaceutical composition or other therapeutic, refers to any lessening, whether permanent or temporary, lasting or transient, of the symptoms that can be attributed to or associated with administration of the composition or therapeutic.

As used herein, the term “subject” refers to an animal, including a mammal, such as a human being.

As used herein, “optional” or “optionally” means that the subsequently described event or circumstance does or does not occur, and that the description includes instances where said event or circumstance occurs and instances where it does not. For example, an optionally substituted group means that the group is unsubstituted or is substituted.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### VIII. EXEMPLARY EMBODIMENTS

Among the embodiments provided herein are:
1. A conjugate comprising an antibody or antigen-binding fragment that specifically binds a CD25 without substantially blocking or interfering with IL-2 signaling, and a Si-phthalocyanine dye; wherein the conjugate is activated by illumination at a wavelength between at or about 600 nm and at or about 850 nm to effect cell killing.
2. The conjugate of embodiment 1, wherein the activated conjugate does not substantially block or interfere with IL-2 signaling.
3. The conjugate of embodiment 1 or 2, wherein the Si-phthalocyanine dye is IR700.
4. The conjugate of embodiment 1 or 2, wherein the Si-phthalocyanine dye has the structure of Formula (I):

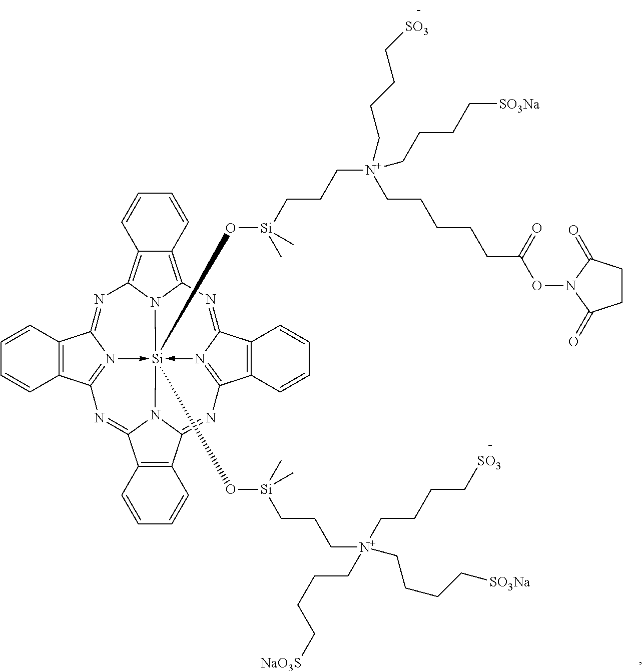

or is a salt, stereoisomer, or tautomer thereof.
5. The conjugate of any of embodiments 1-4, wherein the activated conjugate effects tumor inhibition or killing at a higher level, activity or potency than the unconjugated antibody.
6. The conjugate of any of embodiments 1-5, wherein the antibody or antigen-binding fragment comprises a heavy chain variable (V_{H}) region and a light chain variable (V_{L}) region, wherein:
the V_{H} region comprises a heavy chain complementarity determining region 1 (CDR-H1), a heavy chain complementarity determining region 2 (CDR-H2) and a heavy chain complementarity determining region 3 (CDR-H3) contained within the V_{H} region amino acid sequence set forth in SEQ ID NO: 1 and the V_{L} region comprises a light chain complementarity determining region 1 (CDR-L1), a light chain complementarity determining region 2 (CDR-L2) and a light chain complementarity determining region 3 (CDR-L3) contained within the V_{L} region amino acid sequence set forth in SEQ ID NO: 2;
the V_{H} region comprises a CDR-H1, a CDR-H2 and a CDR-H3 contained within the V_{H} region amino acid sequence set forth in SEQ ID NO: 3 and the V_{L} region comprises a CDR-L1, a CDR-L2 and a CDR-L3 contained within the V_{L} region amino acid sequence set forth in SEQ ID NO: 4;
the V_{H} region comprises a CDR-H1, a CDR-H2 and a CDR-H3 contained within the V_{H} region amino acid sequence set forth in SEQ ID NO: 5 and the V_{L} region comprises a CDR-L1, a CDR-L2 and a CDR-L3 contained within the V_{L} region amino acid sequence set forth in SEQ ID NO: 6;
the V_{H} region comprises a CDR-H1, a CDR-H2 and a CDR-H3 contained within the V_{H} region amino acid sequence set forth in SEQ ID NO: 7 and the V_{L} region comprises a CDR-L1, a CDR-L2 and a CDR-L3 contained within the V_{L} region amino acid sequence set forth in SEQ ID NO: 8;
the V_{H} region comprises a CDR-H1, a CDR-H2 and a CDR-H3 contained within the V_{H} region amino acid sequence set forth in SEQ ID NO: 9 and the V_{L} region comprises a CDR-L1, a CDR-L2 and a CDR-L3 contained within the V_{L} region amino acid sequence set forth in SEQ ID NO: 11;
the V_{H} region comprises a CDR-H1, a CDR-H2 and a CDR-H3 contained within the V_{H} region amino acid sequence set forth in SEQ ID NO: 9 and the V_{L} region comprises a CDR-L1, a CDR-L2 and a CDR-L3 contained within the V_{L} region amino acid sequence set forth in SEQ ID NO: 12;
the V_{H} region comprises a CDR-H1, a CDR-H2 and a CDR-H3 contained within the V_{H} region amino acid sequence set forth in SEQ ID NO: 10 and the V_{L} region comprises a CDR-L1, a CDR-L2 and a CDR-L3 contained within the V_{L} region amino acid sequence set forth in SEQ ID NO: 11;
the V_{H} region comprises a CDR-H1, a CDR-H2 and a CDR-H3 contained within the V_{H} region amino acid sequence set forth in SEQ ID NO: 10 and the V_{L} region comprises a CDR-L1, a CDR-L2 and a CDR-L3 contained within the V_{L} region amino acid sequence set forth in SEQ ID NO: 12;
the V_{H} region comprises a CDR-H1, a CDR-H2 and a CDR-H3 contained within the V_{H} region amino acid sequence set forth in SEQ ID NO: 13 and the V_{L} region comprises a CDR-L1, a CDR-L2 and a CDR-L3 contained within the V_{L} region amino acid sequence set forth in SEQ ID NO: 16;
the V_{H} region comprises a CDR-H1, a CDR-H2 and a CDR-H3 contained within the V_{H} region amino acid sequence set forth in SEQ ID NO: 13 and the V_{L} region comprises a CDR-L1, a CDR-L2 and a CDR-L3 contained within the V_{L} region amino acid sequence set forth in SEQ ID NO: 17;
the V_{H} region comprises a CDR-H1, a CDR-H2 and a CDR-H3 contained within the V_{H} region amino acid sequence set forth in SEQ ID NO: 13 and the V_{L} region comprises a CDR-L1, a CDR-L2 and a CDR-L3 contained within the V_{L} region amino acid sequence set forth in SEQ ID NO: 18;
the V_{H} region comprises a CDR-H1, a CDR-H2 and a CDR-H3 contained within the V_{H} region amino acid sequence set forth in SEQ ID NO: 13 and the V_{L} region comprises a CDR-L1, a CDR-L2 and a CDR-L3 contained within the V_{L} region amino acid sequence set forth in SEQ ID NO: 19;
the V_{H} region comprises a CDR-H1, a CDR-H2 and a CDR-H3 contained within the V_{H} region amino acid sequence set forth in SEQ ID NO: 14 and the V_{L} region comprises a CDR-L1, a CDR-L2 and a CDR-L3 contained within the V_{L} region amino acid sequence set forth in SEQ ID NO: 16;
the V_{H} region comprises a CDR-H1, a CDR-H2 and a CDR-H3 contained within the V_{H} region amino acid sequence set forth in SEQ ID NO: 14 and the V_{L} region comprises a CDR-L1, a CDR-L2 and a CDR-L3 contained within the V_{L} region amino acid sequence set forth in SEQ ID NO: 17;
the V_{H} region comprises a CDR-H1, a CDR-H2 and a CDR-H3 contained within the V_{H} region amino acid sequence set forth in SEQ ID NO: 14 and the V_{L} region comprises a CDR-L1, a CDR-L2 and a CDR-L3 contained within the V_{L} region amino acid sequence set forth in SEQ ID NO: 18;
the V_{H} region comprises a CDR-H1, a CDR-H2 and a CDR-H3 contained within the V_{H} region amino acid sequence set forth in SEQ ID NO: 14 and the V_{L} region comprises a CDR-L1, a CDR-L2 and a CDR-L3 contained within the V_{L} region amino acid sequence set forth in SEQ ID NO: 19;
the V_{H} region comprises a CDR-H1, a CDR-H2 and a CDR-H3 contained within the V_{H} region amino acid sequence set forth in SEQ ID NO: 15 and the V_{L} region comprises a CDR-L1, a CDR-L2 and a CDR-L3 contained within the V_{L} region amino acid sequence set forth in SEQ ID NO: 16;
the V_{H} region comprises a CDR-H1, a CDR-H2 and a CDR-H3 contained within the V_{H} region amino acid sequence set forth in SEQ ID NO: 15 and the V_{L} region comprises a CDR-L1, a CDR-L2 and a CDR-L3 contained within the V_{L} region amino acid sequence set forth in SEQ ID NO: 17;
the V_{H} region comprises a CDR-H1, a CDR-H2 and a CDR-H3 contained within the V_{H} region amino acid sequence set forth in SEQ ID NO: 15 and the V_{L} region comprises a CDR-L1, a CDR-L2 and a CDR-L3 contained within the V_{L} region amino acid sequence set forth in SEQ ID NO: 18; or
the V_{H} region comprises a CDR-H1, a CDR-H2 and a CDR-H3 contained within the V_{H} region amino acid sequence set forth in SEQ ID NO: 15 and the V_{L} region comprises a CDR-L1, a CDR-L2 and a CDR-L3 contained within the V_{L} region amino acid sequence set forth in SEQ ID NO: 19.
7. The conjugate of any of embodiments 1-6, wherein the antibody or antigen-binding fragment comprises a heavy chain variable (V_{H}) region and a light chain variable (V_{L}) region, wherein:
the V_{H} region comprises a heavy chain complementarity determining region 1 (CDR-H1) comprising the amino acid sequence set forth in SEQ ID NO: 20; a heavy chain complementarity determining region 2 (CDR-H2) comprising the amino acid sequence set forth in SEQ ID NO: 21, and a heavy chain complementarity determining region 3 (CDR-H3) comprising the amino acid sequence set forth in SEQ ID NO: 22, and the V_{L} region comprises a light chain complementarity determining region 1 (CDR-L1) comprising the amino acid sequence set forth in SEQ ID NO: 23; a light chain complementarity determining region 2 (CDR-L2) comprising the amino acid sequence set forth in SEQ ID NO: 24, and a light chain complementarity determining region 3 (CDR-L3) comprising the amino acid sequence set forth in SEQ ID NO: 25;
the V_{H} region comprises a CDR-H1 comprising the amino acid sequence set forth in SEQ ID NO: 26; a CDR-H2 comprising the amino acid sequence set forth in SEQ ID NO: 27, and a CDR-H3 comprising the amino acid sequence set forth in SEQ ID NO: 28, and the V_{L} region comprises a CDR-L1 comprising the amino acid sequence set forth in SEQ ID NO: 29; a CDR-L2 comprising the amino acid sequence set forth in SEQ ID NO: 24, and a CDR-L3 comprising the amino acid sequence set forth in SEQ ID NO: 30;
the V_{H} region comprises a CDR-H1 comprising the amino acid sequence set forth in SEQ ID NO: 31; a CDR-H2 comprising the amino acid sequence set forth in SEQ ID NO: 32, and a CDR-H3 comprising the amino acid sequence set forth in SEQ ID NO: 33, and the V_{L} region comprises a CDR-L1 comprising the amino acid sequence set forth in SEQ ID NO: 34; a CDR-L2 comprising the amino acid sequence set forth in SEQ ID NO: 35, and a CDR-L3 comprising the amino acid sequence set forth in SEQ ID NO: 36; or
the V_{H} region comprises a CDR-H1 comprising the amino acid sequence set forth in SEQ ID NO: 37; a CDR-H2 comprising the amino acid sequence set forth in SEQ ID NO: 38, and a CDR-H3 comprising the amino acid sequence set forth in SEQ ID NO: 39, and the V_{L} region comprises a CDR-L1 comprising the amino acid sequence set forth in SEQ ID NO: 40; a CDR-L2 comprising the amino acid sequence set forth in SEQ ID NO: 41, and a CDR-L3 comprising the amino acid sequence set forth in SEQ ID NO: 42.
8. The conjugate of any of embodiments 1-7, wherein the antibody or antigen-binding fragment comprises a heavy chain variable (V_{H}) region and a light chain variable (V_{L}) region, wherein:
the V_{H} region comprises the amino acid sequence set forth in SEQ ID NO: 1 and the V_{L} region comprises the amino acid sequence set forth in SEQ ID NO: 2;
the V_{H} region comprises the amino acid sequence set forth in SEQ ID NO: 3 and the V_{L} region comprises the amino acid sequence set forth in SEQ ID NO: 4;
the V_{H} region comprises the amino acid sequence set forth in SEQ ID NO: 5 and the V_{L} region comprises the amino acid sequence set forth in SEQ ID NO: 6;
the V_{H} region comprises the amino acid sequence set forth in SEQ ID NO: 7 and the V_{L} region comprises the amino acid sequence set forth in SEQ ID NO: 8;
the V_{H} region comprises the amino acid sequence set forth in SEQ ID NO: 9 and the V_{L} region comprises the amino acid sequence set forth in SEQ ID NO: 11;
the V_{H} region comprises the amino acid sequence set forth in SEQ ID NO: 9 and the V_{L} region comprises the amino acid sequence set forth in SEQ ID NO: 12;
the V_{H} region comprises the amino acid sequence set forth in SEQ ID NO: 10 and the V_{L} region comprises the amino acid sequence set forth in SEQ ID NO: 11;
the V_{H} region comprises the amino acid sequence set forth in SEQ ID NO: 10 and the V_{L} region comprises the amino acid sequence set forth in SEQ ID NO: 12;
the V_{H} region comprises the amino acid sequence set forth in SEQ ID NO: 13 and the V_{L} region comprises the amino acid sequence set forth in SEQ ID NO: 16;
the V_{H} region comprises the amino acid sequence set forth in SEQ ID NO: 13 and the V_{L} region comprises the amino acid sequence set forth in SEQ ID NO: 17;
the V_{H} region comprises the amino acid sequence set forth in SEQ ID NO: 13 and the V_{L} region comprises the amino acid sequence set forth in SEQ ID NO: 18;
the V_{H} region comprises the amino acid sequence set forth in SEQ ID NO: 13 and the V_{L} region comprises the amino acid sequence set forth in SEQ ID NO: 19;
the V_{H} region comprises the amino acid sequence set forth in SEQ ID NO: 14 and the V_{L} region comprises the amino acid sequence set forth in SEQ ID NO: 16;
the V_{H} region comprises the amino acid sequence set forth in SEQ ID NO: 14 and the V_{L} region comprises the amino acid sequence set forth in SEQ ID NO: 17;
the V_{H} region comprises the amino acid sequence set forth in SEQ ID NO: 14 and the V_{L} region comprises the amino acid sequence set forth in SEQ ID NO: 18;
the V_{H} region comprises the amino acid sequence set forth in SEQ ID NO: 14 and the V_{L} region comprises the amino acid sequence set forth in SEQ ID NO: 19;
the V_{H} region comprises the amino acid sequence set forth in SEQ ID NO: 15 and the V_{L} region comprises the amino acid sequence set forth in SEQ ID NO: 16;
the V_{H} region comprises the amino acid sequence set forth in SEQ ID NO: 15 and the V_{L} region comprises the amino acid sequence set forth in SEQ ID NO: 17;
the V_{H} region comprises the amino acid sequence set forth in SEQ ID NO: 15 and the V_{L} region comprises the amino acid sequence set forth in SEQ ID NO: 18; or
the V_{H} region comprises the amino acid sequence set forth in SEQ ID NO: 15 and the V_{L} region comprises the amino acid sequence set forth in SEQ ID NO: 19.
9. The conjugate of any of embodiments 1-8, wherein the antibody or antigen-binding fragment comprises MA251, 7G7B6 or an antigen-binding portion thereof.
10. The conjugate of any of embodiments 1-9, wherein the conjugate exhibits one or more Fc-mediated effector function(s).
11. The conjugate of any of embodiments 1-9, wherein the conjugate lacks Fc-mediated effector function(s), exhibits substantially reduced Fc-mediated effector function(s) or does not exhibit substantial Fc-mediated effector function(s).
12. The conjugate of embodiment 11, wherein the activated conjugate is capable of cell killing in the absence of substantial Fc-mediated effector function(s).
13. The conjugate of any of embodiments 10-12, wherein the Fc-mediated effector function is selected from one or more of an antibody-dependent cellular cytotoxicity (ADCC), antibody-dependent cellular phagocytosis (ADCP) or complement-dependent cytotoxicity (CDC).
14. The conjugate of any of embodiments 1-13, wherein the conjugate, when activated has at least two modes of action to effect cell killing.
15. The conjugate of embodiment 14, wherein one of the at least two modes of action is ADCC independent.
16. The conjugate of embodiment 1-15, wherein the activated conjugate exhibits at least one mode of cell killing or tumor inhibition not present in the unconjugated antibody.
17. The conjugate of any of embodiments 1-16, wherein the conjugate comprises an IgG1 Fc region or an IgG1 isotype, an IgG2 Fc region or an IgG2 isotype, IgG3 Fc region or an IgG3 isotype, or an IgG4 Fc region or an IgG4 isotype.
18. The conjugate of any of embodiments 1-17, wherein the antibody or antibody-binding fragment comprises an IgG1 Fc region or an IgG1 isotype.
19. The conjugate of embodiment 18, wherein the IgG1 Fc region does not exhibit enhanced antibody-dependent cellular cytotoxicity (ADCC) effector function.
20. The conjugate of any of embodiments 1-17, wherein the antibody or antibody-binding fragment comprises an IgG2 Fc region or an IgG2 isotype.
21. The conjugate of embodiment 20, wherein the IgG2 Fc region comprises a substitution that reduces or abrogates ADCC effector function.
22. The conjugate of embodiment 21, wherein the substitution is a substitution of glutamine for the asparagine in the Fc region at the position corresponding to 297 according to EU numbering (N297Q).
23. The conjugate of any of embodiments 1-22, wherein the antibody or antigen-binding fragment is a human, chimeric, or humanized antibody or antigen-binding fragment.
24. The conjugate of any of embodiments 1-23, wherein the antibody or antigen binding fragment comprises an Fc region of a human immunoglobulin and/or human antibody framework regions.
25. A method of treating a tumor or a lesion in a subject, comprising:
a) administering to the subject the conjugate of any of embodiments 1-24; and
b) illuminating a target site within the subject with a wavelength of between at or about 600 nm and at or about 850 nm, and at a dose of from at or about 25 J/cm² to at or about 400 J/cm² or from at or about 2 J/cm fiber length to at or about 500 J/cm fiber length, thereby activating the conjugate;
whereby the growth, volume or dimensions of the tumor or the lesion is reduced or inhibited.
26. A method of treating a tumor or a lesion in a subject, comprising:
a) administering to the subject a conjugate, wherein the conjugate comprises an antibody or antigen-binding fragment that specifically binds a CD25 without substantially blocking or interfering with IL-2 signaling, and a Si-phthalocyanine dye; and
b) illuminating a target site within the subject with a wavelength of between at or about 600 nm and at or about 850 nm, and at a dose of from at or about 25 J/cm² to at or about 400 J/cm² or from at or about 2 J/cm fiber length to at or about 500 J/cm fiber length, thereby activating the conjugate;
whereby the growth, volume or dimensions of the tumor or the lesion is reduced or inhibited.
27. The method of embodiment 25 or 26, wherein the tumor or lesion to be treated, or the tumor microenvironment (TME) of the tumor or lesion to be treated, contains reduced levels of immune effector cells.
28. The method of embodiment 26 or 27, wherein the Si-phthalocyanine dye is IR700 and the illuminating step is performed at a wavelength of 690 nm±20 nm.
29. The method of embodiment 26 or 27, wherein the Si-phthalocyanine dye comprises Formula (I)

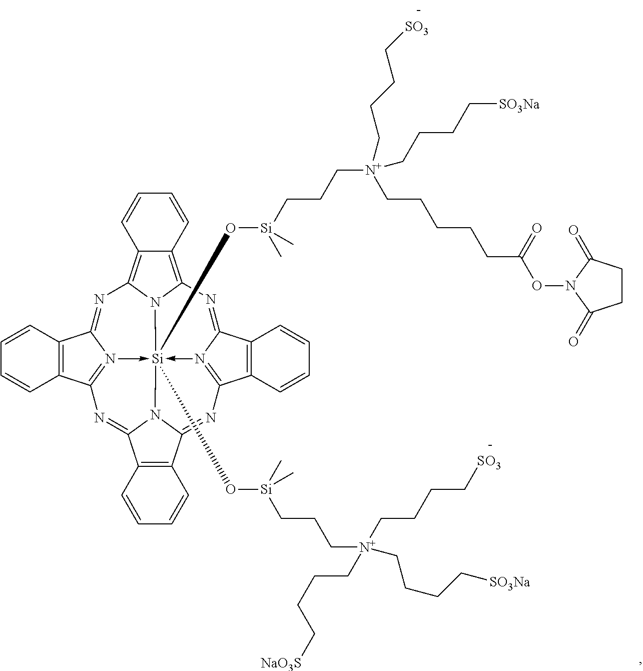

or a salt, stereoisomer, or tautomer thereof, and the illuminating step is performed at a wavelength of 660 nm±50 nm.
30. The method of any of embodiments 27-29, wherein the immune effector cells are selected from one or more of macrophages, natural killer (NK) cells, neutrophils, and eosinophils.
31. The method of any of embodiments 25-30, wherein the target site is illuminated within about 24±4 hours after administering the conjugate.
32. The method of any of embodiments 25-31, wherein the target site is illuminated at an optical power of between at or about 50 mW/cm² and at or about 200 mW/cm².
33. The method of any of embodiments 25-31, wherein the target site is illuminated at an optical power of between at or about 100 mW/cm fiber length and at or about 500 mW/cm fiber length.
34. The method of any of embodiments 25-33, wherein the target site is illuminated for between at or about 120 seconds and at or about 600 seconds.
35. The method of any of embodiments 25-34, wherein the tumor or lesion is resistant or non-responsive to immune checkpoint inhibitor therapy.
36. The method of any of embodiments 25-34, wherein the tumor or lesion has a reduced response or is non-responsive to the unconjugated antibody.
37. The method of any of embodiments 26-36, wherein the conjugate exhibits one or more Fc-mediated effector function(s).
38. The method of any of embodiments 26-36, wherein the conjugate lacks Fc-mediated effector function(s), exhibits substantially reduced Fc-mediated effector function(s) or does not exhibit substantial Fc-mediated effector function(s).
39. The method of embodiment 38, wherein the activated conjugate is capable of cell killing in the absence of substantial Fc-mediated effector function(s).
40. The method of any of embodiments 37-39, wherein the Fc-mediated effector function is selected from one or more of an antibody-dependent cellular cytotoxicity (ADCC), antibody-dependent cellular phagocytosis (ADCP) or complement-dependent cytotoxicity (CDC).
41. The method of any of embodiments 26-40, wherein the conjugate comprises an IgG1 Fc region or an IgG1 isotype, an IgG2 Fc region or an IgG2 isotype, IgG3 Fc region or an IgG3 isotype, or an IgG4 Fc region or an IgG4 isotype.
42. The method of any of embodiments 26-41, wherein the antibody or antibody-binding fragment comprises an IgG1 Fc region or an IgG1 isotype.
43. The method of embodiment 42, wherein the IgG1 Fc region is not enhanced for ADCC effector function.
44. The method of any of embodiments 26-43, wherein the antibody or antibody-binding fragment comprises an IgG2 Fc region or an IgG2 isotype.
45. The method of embodiment 44, wherein the IgG2 Fc region comprises a substitution that reduces or abrogates ADCC effector function.
46. The method of embodiment 45, wherein the substitution is a substitution of glutamine for the asparagine in the Fc region at the position corresponding to 297 according to EU numbering (N297Q).
47. The method of any of embodiments 25-46, further comprising administering an immune checkpoint inhibitor therapy subsequent to the administration of the conjugate.
48. The method of embodiment 47, wherein the immune checkpoint inhibitor therapy is administered 1 day, 2 days, 3 days, 4 days, 5 days, 6 days, 7 days, 1 week, 2 weeks or 3 weeks following administration of the conjugate.
49. The method of embodiment 47 or 48, wherein the immune checkpoint inhibitor therapy is administered 1 day, 2 days, 3 days, 4 days, 5 days, 6 days, 7 days, 1 week, 2 weeks or 3 weeks following the illumination.
50. The method of any of embodiments 47-49, wherein the immune checkpoint inhibitor therapy is administered more than once following administration of the conjugate.
51. The method of any of embodiments 47-50, wherein the immune checkpoint inhibitor therapy comprises a PD-1 inhibitor, a PD-L1 inhibitor or a CTLA-4 inhibitor.
52. The method of embodiment 51, wherein the PD-1 inhibitor is an anti-PD-1 antibody selected from pembrolizumab (MK-3475, KEYTRUDA; lambrolizumab), nivolumab (OPDIVO), cemiplimab (LIBTAYO), toripalimab (JS001), HX008, SG001, GLS-010, dostarlimab (TSR-042), tislelizumab (BGB-A317), cetrelimab (JNJ-63723283), pidilizumab (CT-011), genolimzumab (APL-501, GB226), BCD-100, cemiplimab (REGN2810), F520, sintilimab (IB1308), CS1003, LZM009, camrelizumab (SHR-1210), SCT-I10A, MGA012, AK105, PF-06801591, AMP-224, AB122, AMG 404, BI 754091, HLX10, JTX-4014, AMP-514 (MEDI0680), Sym021, MGD019, MGD013, AK104, XmAb20717, R07121661, CX-188, spartalizumab, BCD-217, HX009, IBI308, PDR001, REGN2810, TSR-042 (ANB011), or an antigen-binding fragment thereof or any combination thereof.
53. The method of any of embodiments 25-52, wherein the population of regulatory T cells (Tregs) in the tumor or the lesion or in the tumor microenvironment is reduced as a result of the method.
54. The method of any of embodiments 25-53, wherein the reduction or inhibition comprises one or more of less than 20% increase in tumor volume or tumor dimensions, or a reduction in tumor volume, tumor dimensions or tumor mass, or a reduction in number of tumor cells.
55. The method of any of embodiments 25-54, wherein the growth, volume or dimensions of the tumor or the lesion is inhibited or reduced to a greater degree as compared to a method that employs a conjugate comprising an antibody or antigen-binding fragment that specifically binds a CD25 and substantially blocks or interferes with IL-2 signaling.
56. The method of any of embodiments 25-55, wherein the method improves the survival of the subject.
57. The method of any of embodiments 25-56, wherein the subject comprises a second tumor or secondary population of tumor cells, and wherein the growth, volume or dimensions of the second tumor or secondary population of tumor cells is reduced or inhibited as a result of the method.
58. The method of embodiment 57, wherein the second tumor or secondary population of tumor cells has not been illuminated.

### IX. EXAMPLES

The following examples are included for illustrative purposes only and are not intended to limit the scope of the invention.

### Example 1: Generation of IRDye 700-Conjugated Anti-CD25 Antibodies

This example describes a method for preparing a conjugate containing an exemplary phthalocyanine dye IRDye 700DX (IR700) linked to mouse IgG1 and IgG2a isotypes of anti-CD25 antibodies PC61 (exemplary IL-2 blocking anti-CD25 antibody) and 7D4 (exemplary IL-2 non-blocking anti-CD25 antibody), producing PC61-mIgG1-IR700, PC61-mIgG2a-IR700, 7D4-mIgG1-IR700, and 7D4-mIgG2a-IR700. The corresponding SEQ ID NOS for the amino acid sequences of the variable heavy chain (VH), constant heavy chain (CH1-CH3), variable light chain (VL), and constant light chain (CL) of the antibodies are designated in Table 2.

TABLE 2

| | Variable | Constant | Variable | Constant |
|---|---|---|---|---|
| | heavy chain | heavy chain | light chain | light chain |
| | (SEQ | (SEQ | (SEQ | (SEQ |
| Antibody | ID NO) | ID NO) | ID NO) | ID NO) |
| 7D4-mIgG1 | 43 | 44 | 46 | 47 |
| 7D4-mIgG2a | 43 | 45 | 46 | 47 |
| PC61-mIgG1 | 48 | 49 | 50 | 51 |
| PC61-mIgG2a | 48 | 45 | 50 | 51 |

PC61 or 7D4, rat monoclonal antibodies (mAb) directed against mouse CD25, of isotype mIgG1 or mIgG2a, were incubated with IRDye 700DX NHS Ester (IR700; LI-COR Bioscience, Lincoln, NE) (52.1-71.9 μg, [27.2-36.8 nmol], 5 mmol/L in DMSO for each 1 mg [6.8 nmol] antibody) in 0.1 mol/L Na₂HPO₄ (pH 8.5) at room temperature for 30 to 120 min. Conjugates were prepared in batches ranging from 50-182 mg of antibody. The mixture was then spiked with 1 mol/L C₂H₅NO₂ (pH 9.0) to a target of 20 mmol/L at room temperature for 2-16 hr. The mixture was buffer exchanged using Amicon centrifugal spin filters (30 kD; Millipore Sigma). Protein concentration and dye-to-antibody ratio (DAR) were determined with analytical size-exclusion (SEC-HPLC) by measuring the absorption at 280 nm and 690 nm. Protein concentration was calculated using the absorption at 280 nm corrected for the effects of dye absorption at 690 nm. Average DAR was calculated as a ratio between the peak areas of the wavelengths measured. The average number of IR700 per mAb (PC61 or 7D4) was about 3.

Purity of the PC61-IR700 and 7D4-IR700 conjugates was confirmed by analytical size-exclusion HPLC (SE-HPLC). SE-HPLC was performed using an Agilent 1100 HPLC system (Santa Clara, CA) equipped with a PDA detector controlled by Chemstation software. SE chromatography was performed on a Shodex KW-803 column (New Yok, NY) eluted for 20 minutes using phosphate buffered saline (PBS) at 1.0 mL/min. The conjugate preparations exhibited strong association and contained no detectable mAb aggregates as determined by SE-HPLC.

To determine the in vitro binding characteristics of IR700 conjugates, ¹²⁵I labeling of the conjugates using the Indo-Gen procedure was performed. Minimal loss of mAb with IR700 conjugation was observed. Immunoreactivity assay was performed. Briefly, after trypsinization, 2×10⁶ of tumor cells were resuspended in PBS containing 1% bovine serum albumin (BSA). ¹²⁵I-PC61-IR700 (1 mCi, 0.2 μg) or ¹²⁵I-7D4-IR700 (1 mCi, 0.2 μg) was added and incubated for 1 hour on ice. The cells were washed, pelleted, the supernatant was decanted, and the cells were counted in a 2470 Wizard gamma-counter (Perkin Elmer, Shelton, CT). Nonspecific binding to the cells was examined under conditions of excess unlabeled antibody (200 μg of the unconjugated, non-labeled mAb).

### Example 2: Photoimmunotherapy (PIT) Using IL-2 Non-Blocking Anti-CD25 Conjugates Is More Effective at Inhibiting Tumor Growth Than IL-2 Blocking PIT

This example compares the activity of an exemplary IL-2 non-blocking anti-CD25-IR700 conjugate and an exemplary IL-2 blocking anti-CD25-IR700 conjugate, with and without light illumination on primary colon carcinoma tumors.

BALB/c mice at the age of 6-8 weeks were inoculated with 1×10⁶ CT26 murine colon carcinoma cells subcutaneously in the right hind flank. When allograft tumors grew to a size of about 150 mm³ (approximately day 6 after tumor implantation) mice were administered saline (100 μL; n=12; control), IL-2 non-blocking anti-CD25 antibody 7D4-mIgG1-IR700 conjugate (7D4-IR700; 100 μg; n=24), or IL-2 blocking anti-CD25 antibody PC61-IgG1-IR700 conjugate (PC61-IR700; 100 μg; n=24), generated as described in Example 1 above, by retro-orbital (RO) injection. Twenty-four hours after administration of the conjugate, tumors in half of the mice administered a conjugate (photoimmunotherapy (PIT) groups) were illuminated at 690 nm and a dosage of 100 J/cm². Tumor growth for all mice was measured every 2-3 days, and tumor volume was calculated using the formula: tumor volume=(width×width)×length/2. Survival of the mice was also recorded over time.

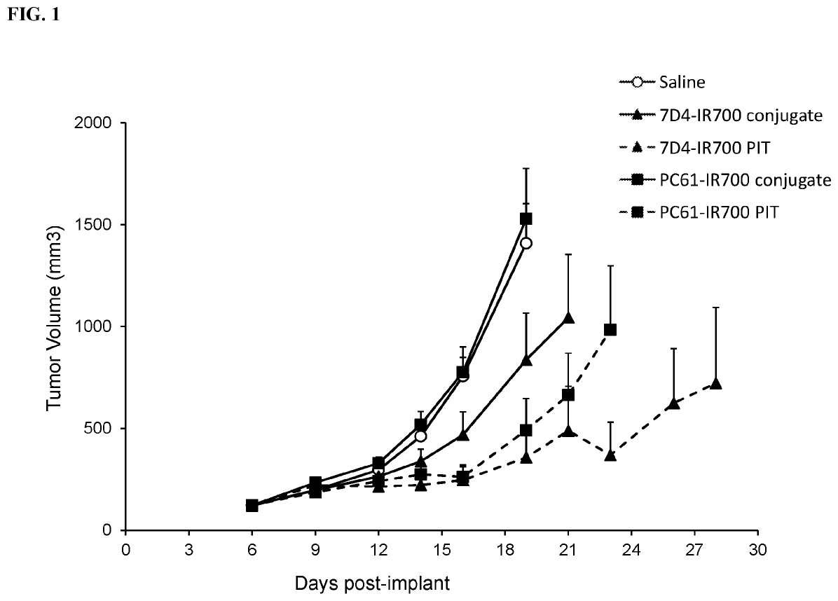

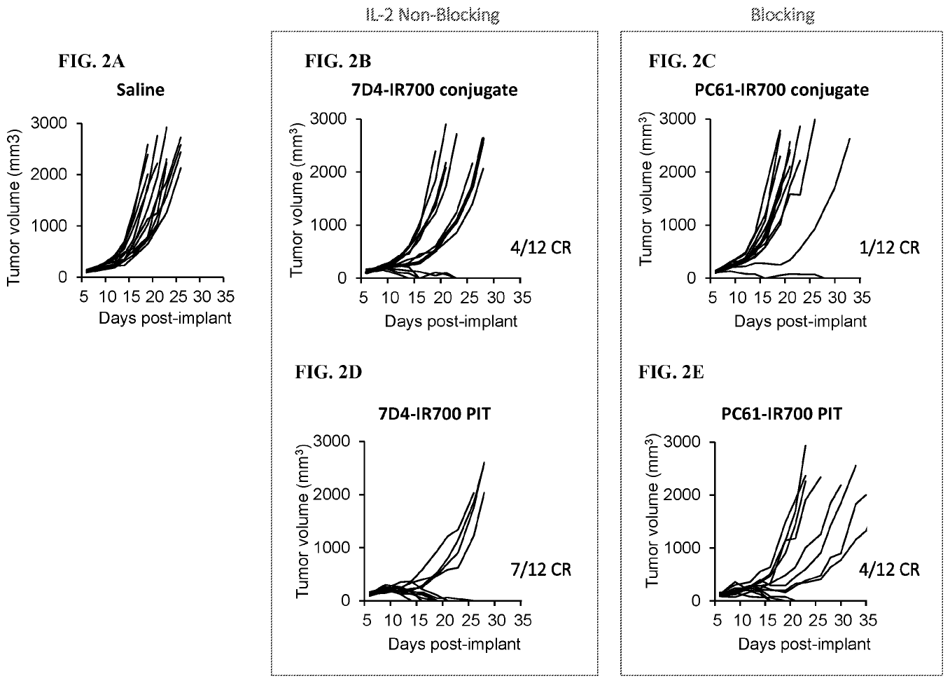

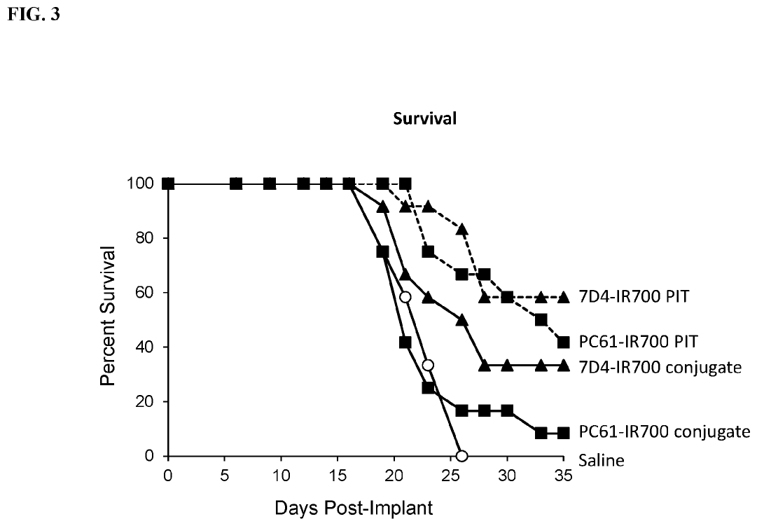

Average tumor growth and growth of individual tumors, for each treatment group, are shown in FIG. 1 and FIGS. 2A-2E, respectively. Mice administered the exemplary IL-2 non-blocking anti-CD25-IR700 conjugate combined with illumination (7D4-IR700 PIT; filled triangles, dashed line), the average growth of tumors was substantially inhibited in comparison to average tumor growth inhibition in control mice that received saline (open circles) or 7D4-IR700 conjugate alone without illumination (filled triangles, solid line). Mice administered 7D4-IR700 conjugate alone without illumination also exhibited a reduction in tumor growth compared to saline control mice. In contrast, the average tumor growth in mice administered the IL-2 blocking anti-CD25-IR700 conjugate alone, without illumination (PC61-IR700 conjugate) was indistinguishable from saline control mice (filled squares, solid line vs. open circles). Tumors in mice administered IL-2 blocking anti-CD25-IR700 conjugate and illumination (PC61-IR700 PIT; filled squares, dashed line) exhibited a reduction in tumor growth that was more than IL-2 non-blocking anti-CD25 conjugate alone, but less than the reduction in tumor growth observed for IL-2 non-blocking anti-CD25 PIT. As shown in FIGS. 2A-2E, plotting the growth of individual tumors, 7/12 mice administered IL-2 non-blocking anti-CD25 PIT (7D4-IR700 PIT) achieved complete response (CR) (FIG. 2D), while 4/12 mice administered IL-2 non-blocking anti-CD25 conjugate alone or administered IL-2 blocking anti-CD25 PIT achieved CR (FIG. 2B and FIG. 2E, respectively), 1/12 mice administered IL-2 blocking anti-CD25 conjugate alone achieved CR (FIG. 2C), and 0/12 control mice, administered saline, achieved CR (FIG. 2A). Consistent with these results, and shown in FIG. 3, mice administered IL-2 non-blocking anti-CD25 PIT (7D4-IR700 PIT) exhibited the greatest survival (filled triangles, dashed line), followed by administered IL-2 blocking anti-CD25 PIT (PC61-IR700 PIT; filled squares, dashed line), IL-2 non-blocking anti-CD25 conjugate alone (no illumination) (7D4-IR700 conjugate; filled triangles, solid line), IL-2 blocking anti-CD25 conjugate alone (PC61-IR700 conjugate; filled squares, solid line), and saline control (saline; open circles, solid line). Taken together, these results indicate IL-2 non-blocking anti-CD25 PIT is more effective than IL-2 blocking anti-CD25 PIT, and more effective than IL-2 non-blocking anti-CD25 conjugate alone in inhibiting tumor growth and promoting survival in tumor-bearing mice.

### Example 3: IL-2 Non-Blocking Anti-CD25 Photoimmunotherapy (PIT) and Anti-PD-1 Antibody Synergistically Inhibit Tumor Growth In Vivo

This example describes the synergistic inhibitory effect of an exemplary IL-2 non-blocking anti-CD25-IR700 photoimmunotherapy (PIT) in combination with an anti-PD-1 antibody on tumor growth in an anti-PD-1 resistant mouse tumor model, compared to treatment with a naked (unconjugated) anti-CD25 antibody.

Immunocompetent BALB/c mice at the age of 6-8 weeks were inoculated with 5×10⁵ MCA205 murine fibrosarcoma cells, subcutaneously in the right hind flank (Day 0). When allograft tumors grew to a size of about 150 mm³ (Day 7) mice were administered saline (Group 1), anti-PD-1 antibody (Group 2), naked IL-2 non-blocking 7D4-mIgG2a antibody (Group 3), 7D4-mIgG2a-IR700 conjugate (Group 4), naked 7D4-mIgG2a antibody and anti-PD-1 antibody (Group 5), or 7D4-mIgG2a-IR700 conjugate and anti-PD-1 antibody (Group 6) as specified in Table 3. The conjugates were generated as described in Example 1. Saline, antibodies, and conjugates were administered via retro-orbital (RO) injection. Twenty-four hours after administration of the conjugate (Day 8), tumors in half of the mice administered the 7D4-mIgG2a-IR700 conjugate (photoimmunotherapy (PIT) groups) were illuminated at 690 nm and a dosage of 200 J/cm² as indicated in Table 3. Mice in Groups 2, 5, and 6 were administered an anti-PD-1 antibody on Day 7 and received repeated dosing 3 times weekly throughout the study. Tumor growth for all mice was measured every 2-3 days, and tumor volume was calculated using the formula: tumor volume=(width×width)×length/2. Survival was also recorded.

TABLE 3
Treatment Groups

| | | | Conjugate | | Light | | αPD-1 | |
|---|---|---|---|---|---|---|---|---|
| Group | Name | n | Dose | Schedule | Dose | Schedule | Dose | Schedule |
| 1 | Saline | 10 | — | — | — | — | — | — |
| 2 | — | 10 | 100 μg | — | — | — | 100 μg | 3x/wk |
| 3 | 7D4-mIgG2a | 10 | 100 μg | D 7 | — | — | — | — |
| 4 | 7D4-mIgG2a-IR700 | 10 | 100 μg | D 7 | 200 J/cm² | D 8 | — | — |
| 5 | 7D4-mIgG2a | 10 | 100 μg | D 7 | — | — | 100 μg | 3x/wk |
| 6 | 7D4-mIgG2a-IR700 | 10 | 100 μg | D 7 | 200 J/cm² | D 8 | 100 μg | 3x/wk |

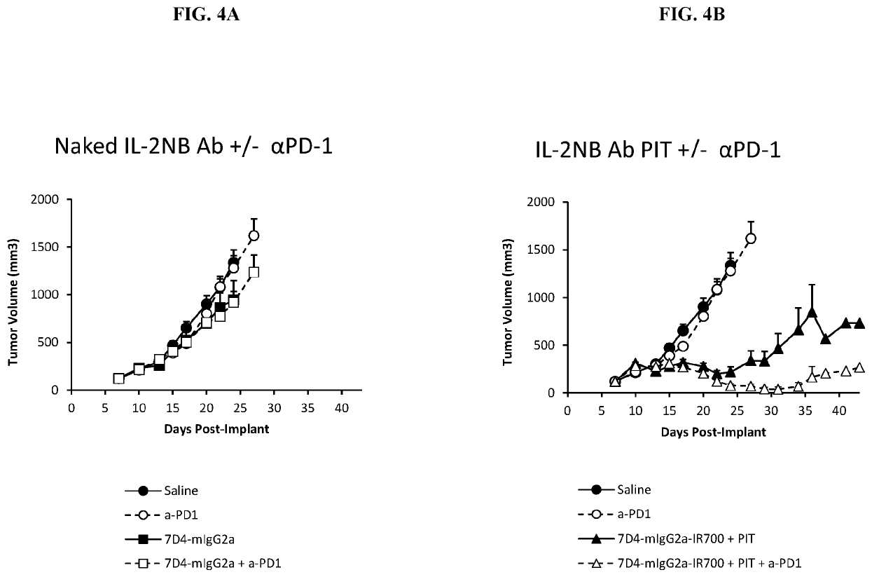

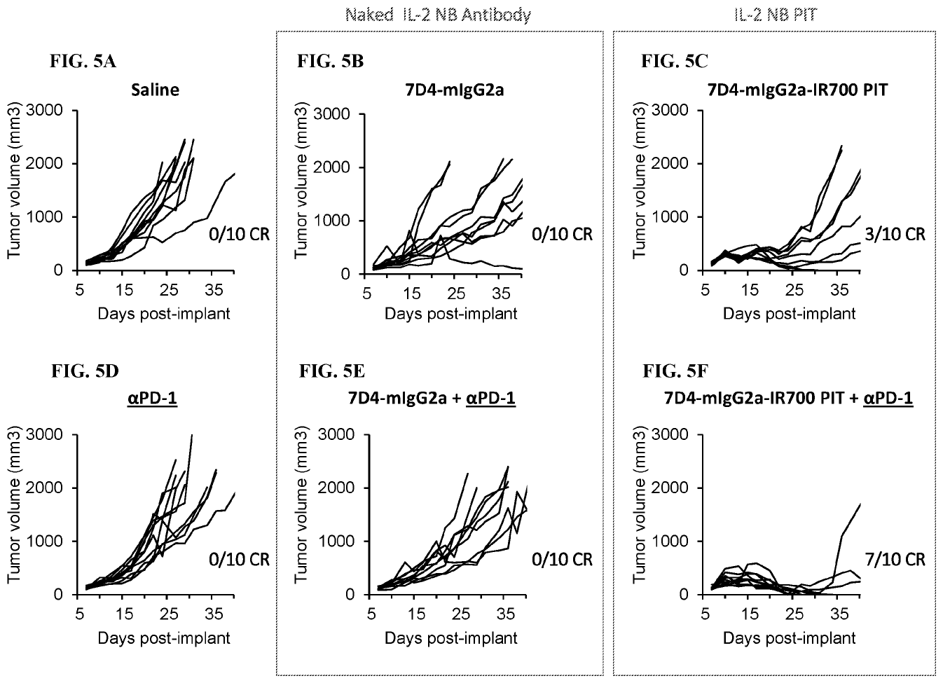

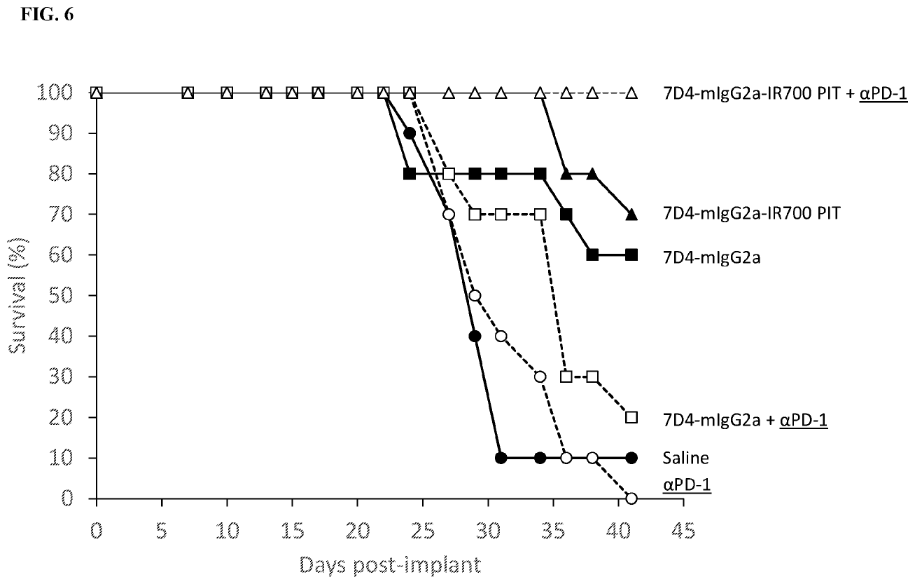

Average tumor growth and growth of individual tumors, for each treatment group, are shown in FIGS. 4A-4B and FIGS. 5A-5E, respectively. FIG. 6 depicts survival. As shown in FIG. 4A and FIGS. 5A, 5D, 5B, and 5E, administration of saline (FIG. 4A; filled circle, solid line and FIG. 5A), anti-PD-1 antibody alone (FIG. 4A; open circle, dashed line and FIG. 5D), naked 7D4-mIgG2a antibody (FIG. 4A; filled square, solid line and FIG. 5B), or naked 7D4-mIgG2a antibody and anti-PD-1 antibody (FIG. 4A; open square, dashed line and FIG. 5E) were ineffective in reducing tumor growth, with no mice achieving CR in any of those treatment groups. These results were also consistent with survival results, where only 7D4-mIgG2a antibody treatment improved survival (FIG. 6; filled squares, solid line). These results indicate MCA205 cells are resistant to anti-PD-1 immunotherapy and IL-2 non-blocking anti-CD25 antibody immunotherapy, either as monotherapies or in combination. In contrast, 7D4-mIgG2a-IR700 PIT substantially reduced tumor growth (FIG. 4B; filled triangles, solid line and FIG. 5C), with 3/10 mice achieving CR, and substantially improved survival (FIG. 6; filled triangles, dashed line), compared to saline controls. The combination of 7D4-mIgG2a-IR700 PIT and anti-PD-1 inhibited tumor growth even further (FIG. 4B; open triangles, dashed line and FIG. 5E), with 7/10 mice achieving CR, and resulted in 100% survival (FIG. 6; open triangle, dashed line). Taken together, these results indicate IL-2 non-blocking anti-CD25 PIT is more effective in inhibiting tumor growth and promoting survival than naked IL-2 non-blocking anti-CD25 antibody alone. Further, the IL-2 non-blocking anti-CD25 PIT synergized with anti-PD-1 treatment to effect even greater inhibition of tumor growth compared to either of the monotherapies alone. IL-2 non-blocking anti-CD25 PIT combined with anti-PD-1 treatment was more effective in inhibiting tumor growth and promoting survival than naked IL-2 non-blocking anti-CD25 antibody treatment, with or without anti-PD-1 treatment. In this anti-PD-1 resistant tumor model, treatment with a naked IL-2 non-blocking anti-CD25 antibody was not effective in reducing tumor growth. Administering anti-PD-1 antibody in addition to the naked IL-2 non-blocking anti-CD25 antibody treatment had no substantial effect in inhibiting tumor growth or promoting survival in this anti-PD-1 resistant model.

### Example 4: Photoimmunotherapy (PIT) Using IL-2 Non-Blocking Anti-CD25 Conjugates is More Effective at Inducing Systemic Immune Responses than IL-2 Blocking PIT

In this example, photoimmunotherapy (PIT) using a conjugate using an IL-2 blocking anti-CD25 antibody as a targeting molecule is compared to PIT using a conjugate using an IL-2 non-blocking anti-CD25 antibody as a targeting molecule for effects on directly treated and non-illuminated, distantly located tumors (abscopal effects).

Immunocompetent BALB/c mice were inoculated with 1×10⁶ MCA-205 murine fibrosarcoma cells subcutaneously on both the right and left hind flanks. When allograft tumors on both sides grew to a volume of about 150 mm³ (5 days after tumor cell inoculation), the mice were intravenously administered saline (100 μL), an exemplary IL-2 blocking anti-CD25 antibody conjugated to IR700 (PC61-mIgG1-IR700; 100 μg) or an exemplary IL-2 non-blocking anti-CD25 antibody conjugated to IR700 (7D4-mIgG1-IR700; 100 μg), generated as described in Example 1 above. Twenty-four hours after administration of the conjugate, tumors in the right flank of half of the animals administered PC61-IR700 or 7D4-IR700 were illuminated at 690 nm at a dosage of 200 J/cm², while tumors in the left flank were shielded from illumination. The growth of the illuminated tumor (target tumor) and the non-illuminated tumor (distal tumor) was observed time, and tumor volume was calculated using the formula: tumor volume=(width×length)×height/2.

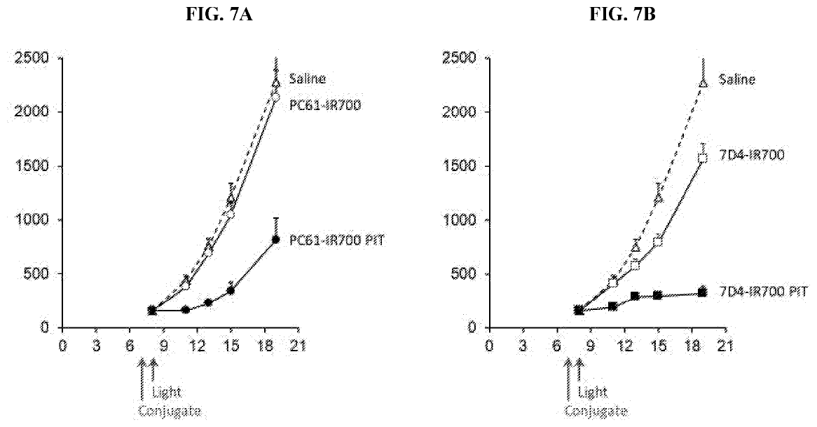

As shown in FIGS. 7A and 7B, tumors of saline-treated animals exhibited rapid growth. Tumor growth in mice treated with IL-2 blocking, PC61-IR700 conjugate alone (no illumination) was indistinguishable from that observed for saline control animals (FIG. 7A; open circles), while tumor growth animals receiving PC61-IR700 PIT exhibited substantial inhibition of tumor growth of the illuminated tumor (FIG. 7A; closed circles). Tumor growth in mice treated with the IL-2 non-blocking 7D4-IR700 conjugate alone (no illumination) exhibited slightly reduced tumor growth compared to the saline controls (FIG. 7B; open squares). Tumors treated with 7D4-IR700 PIT exhibited almost complete suppression of tumor growth of the illuminated tumor following treatment (FIG. 7B; closed squares).

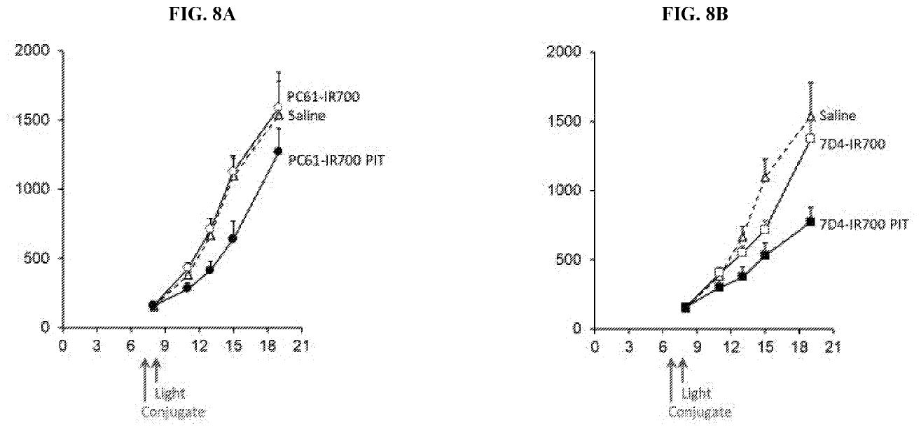

Non-illuminated tumors, distal from the site of illumination (distal tumors), in mice treated with saline (FIGS. 8A and 8B; open triangles) and in mice treated with IL-2 blocking PC61-IR700 conjugate alone (FIG. 8A; open circles) exhibited continuous tumor growth. Non-illuminated, distal tumors in mice treated with PC61 PIT (FIG. 8A; closed circles) or IL-2 non-blocking 7D4 conjugate alone (no illumination) (FIG. 8B; open squares) exhibited a small reduction in tumor growth compared to saline controls. Non-illuminated, distal tumors in mice treated with IL-2 non-blocking 7D4-IR700 conjugate PIT exhibited about 50% reduction in tumor growth compared to saline controls (FIG. 8B; closed squares). The results show that treatment with an anti-CD25 PIT, and in particular, IL-2 non-blocking anti-CD25 PIT, is effective in reducing tumor growth of illuminated tumor lesions and non-illuminated, distal tumor lesions. Based on the abscopal effect (i.e., tumor growth inhibition of the distal, non-illuminated lesion), these results indicate that IL-2 non-blocking anti-CD25 PIT is more effective at inducing systemic immune responses than IL-2 blocking anti-CD25 PIT.

Furthermore, the results indicate that IL-2 non-blocking anti-CD25 PIT treatment is more effective at inhibiting tumor growth in illuminated target lesions and non-illuminated distal lesions than the conjugate treatment alone without illumination.

### Example 5: Antibody-Dependent Cellular Cytotoxicity (ADCC) of an IL-2 Non-Blocking Anti-CD25 Antibody and the Corresponding IL-2 Non-Blocking Anti-CD25 Conjugate

In this example, the antibody-dependent cellular cytotoxicity (ADCC) activity of an exemplary naked (unconjugated) IL-2 non-blocking anti-CD25 antibody and a conjugate comprising the IL-2 non-blocking anti-CD25 antibody and a phthalocyanine dye IR700 was assessed.

ADCC activity was measured for the naked IL-2 non-blocking anti-CD25 antibody (7D4-mIgG2a) and the IL-2 non-blocking anti-CD25-IR700 conjugate (7D4-mIgG2a-IR700), generated as described in Example 1 above, using an ADCC Reporter Bioassay Kit (Promega), substantially as described in the vendor's protocol. Briefly, target HT-2 cells were seeded into a 96-well white-wall plate at a density of 25,000 cells per well. The following day, the culture medium was replaced with 25 μL ADCC Assay Buffer, followed by 25 μL of 9 concentrations of serially diluted 7D4-mIgG2a antibody or 7D4-mIgG2a-IR700 conjugate at a concentration 3 times as high as the final concentration. The serial dilutions were prepared starting from 6 μg/mL (1×=2 μg/mL). The ADCC response induction was initiated by adding 25 μL effector cells (75,000 cells) to achieve an effector to target cell ratio of 3:1. The mixture was incubated for 6 hours at 37° C. To quantify the ADCC response, the plates were brought to room temperature, and 75 μL Bio-Glo Luciferase Assay Reagent were added to the wells containing cells and to three cell-free wells in the perimeter for background determination. After incubation of 20 minutes at ambient temperature, luminescence was read on a Tecan Spark multi-plate reader (Tecan Life Sciences). To analyze data, the background from the cell-free wells was subtracted, and the data were fit to a 4-parameter non-linear curve-fit algorithm (GraphPad Prism software).

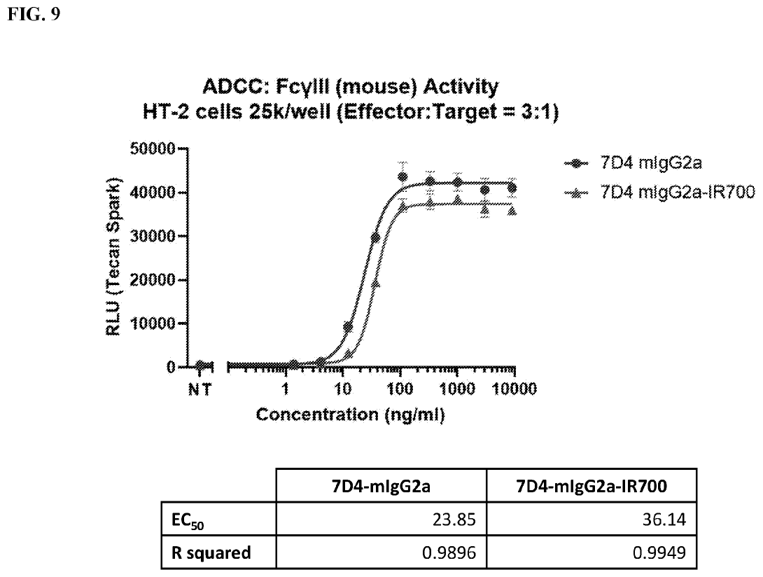

The results are shown in FIG. 9. Both the naked antibody and the conjugate exhibited dose-dependent increases in ADCC activity. The naked 7D4-mIgG2a exhibited stronger ADCC potency and efficacy, with an EC₅₀ of 23.85 (R²: 0.9896), than the 7D4-mIgG2a-IR700 conjugate, which had an EC₅₀ of 36.14 (R²: 0.9949), and a higher saturation point was exhibited by the naked 7D4-mIgG2a antibody compared to the 7D4-mIgG2a-IR700 conjugate. These results indicate that the IR700 conjugate exhibits weaker ADCC activity compared to the naked antibody.

### Example 6: IL-2 Non-Blocking Anti-CD25 Photoimmunotherapy (PIT) does not Require Antibody-Dependent Cellular Cytotoxicity (ADCC)/Antibody-Dependent Cellular Phagocytosis (ADCP) for Anti-tumor Activity

This example evaluates the effect of antibody-dependent cellular cytotoxicity (ADCC) and antibody-dependent cellular phagocytosis (ADCP) on the anti-tumor activity of treatment with an exemplary IL-2 non-blocking anti-CD25 antibody and IL-2 non-blocking anti-CD25 photoimmunotherapy (PIT).

The anti-tumor activity of ADCC/ADCP competent and ADCC/ADCP null IL-2 non-blocking anti-CD25 antibodies on an mIgG2a backbone, and the IL-2 non-blocking anti-CD25 conjugate on an mIgG1 backbone, with and without illumination, were compared using murine primary colon carcinoma tumors. The mIgG2a isotype has a higher binding affinity for the mouse Fc gamma receptor than the mIgG1 isotype and exhibits a higher level of ADCC/ADCP activity than the mIgG1 isotype (Stewart et al., (2014) J. Immunotherapy Cancer 2, 29). Substitution of glutamine for the asparagine in the Fc region at the position corresponding to 297 according to EU numbering (N297Q) abrogates the interaction of the Fc region with the Fc gamma receptor and renders the IgG2a isotype null for ADCC/ADCP activity.

BALB/c mice at the age of 6-8 weeks were inoculated with 1×10⁶ CT26 murine colon carcinoma cells subcutaneously in the right hind flank. When allograft tumors grew to a size of about 150 mm³ (approximately day 6 after tumor implantation) mice were administered saline (100 μL; n=12; control) or IL-2 non-blocking anti-CD25 antibody 7D4-mIgG1-IR700 conjugate (7D4-IR700; 100 μg; n=24), generated as described in Example 1 above, by retro-orbital (RO) injection; or naked (unconjugated) IL-2 non-blocking anti-CD25 antibodies 7D4-mIgG2a (10 mg/kg; n=11) or 7D4-mIgG2a-ADCC/ADCP null (7D4-mIgG2a-N297Q; 10 mg/kg; n=12), by intraperitoneal (IP) administration. Twenty-four hours after administration of the antibody or conjugate, tumors in half of the mice administered a conjugate (photoimmunotherapy (PIT) groups) were illuminated at 690 nm and a dosage of 100 J/cm². Tumor growth for all mice was measured every 2-3 days, and tumor volume was calculated using the formula: tumor volume=(width×width)×length/2. Survival of the mice was also recorded over time.

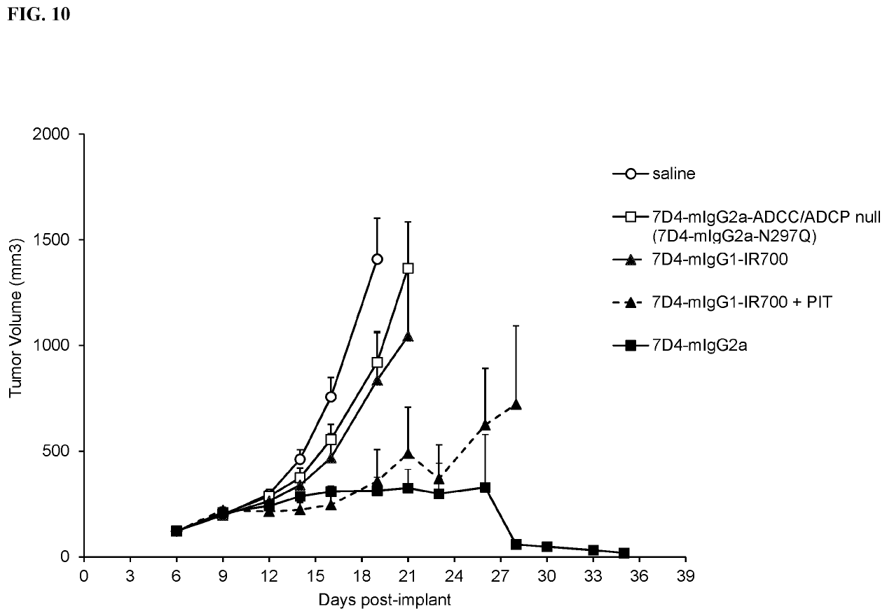

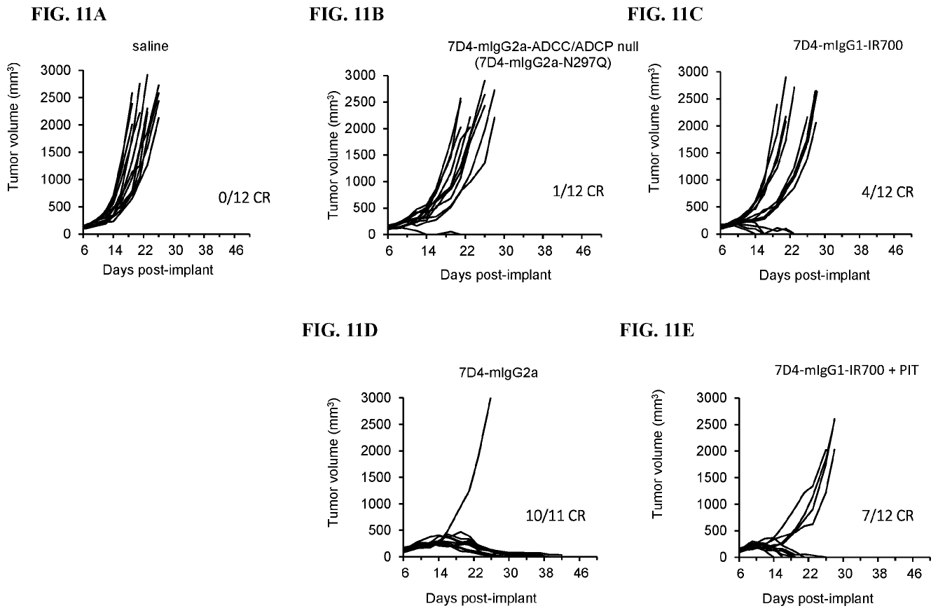

Average tumor growth and growth of individual tumors, for each treatment group, are shown in FIG. 10 and FIGS. 11A-11E, respectively. Mice administered the naked 7D4-mIgG2a antibody exhibited substantial tumor growth inhibition compared to saline-control mice (FIG. 10; closed squares vs. open circles), with 10 of 11 mice achieving complete response (FIG. 11D). This effect was almost completely abrogated in mice administered 7D4-mIgG2a-ADCC/ADCP null (same naked antibody in the context of an ADCC null mutant mIgG2a backbone; FIG. 10; open squares and FIG. 11B), indicating the tumor growth inhibition observed for 7D4-mIgG2a was dependent on ADCC activity.

Mice administered 7D4-mIgG1-IR700 conjugate, without illumination, exhibited similar average tumor growth as the unconjugated 7D4-mIgG2a antibody lacking ADCC/ADCP activity (FIG. 10; closed triangles, solid line (conjugate) vs. open squares, solid line (ADCC/ADCP null antibody)). However, 4 of 12 mice that received 7D4-mIgG1-IR700 conjugate achieved complete responses (FIG. 11C) in comparison to 1 of 12 mice that received 7D4-mIgG2a-N297Q (ADCC null) (FIG. 11B). Because mIgG1 antibodies still exhibit low level of ADCC/ADCP activity, this result further supports the idea that tumor growth inhibition is dependent on ADCC/ADCP activity. Mice administered 7D4-mIgG1-IR700 followed by illumination (7D4-mIgG1-IR700+PIT), however, exhibited substantially more tumor growth inhibition than the conjugate without illumination (FIG. 10 closed triangles, dashed line), with 7 of 12 mice achieving CR (FIG. 11E).

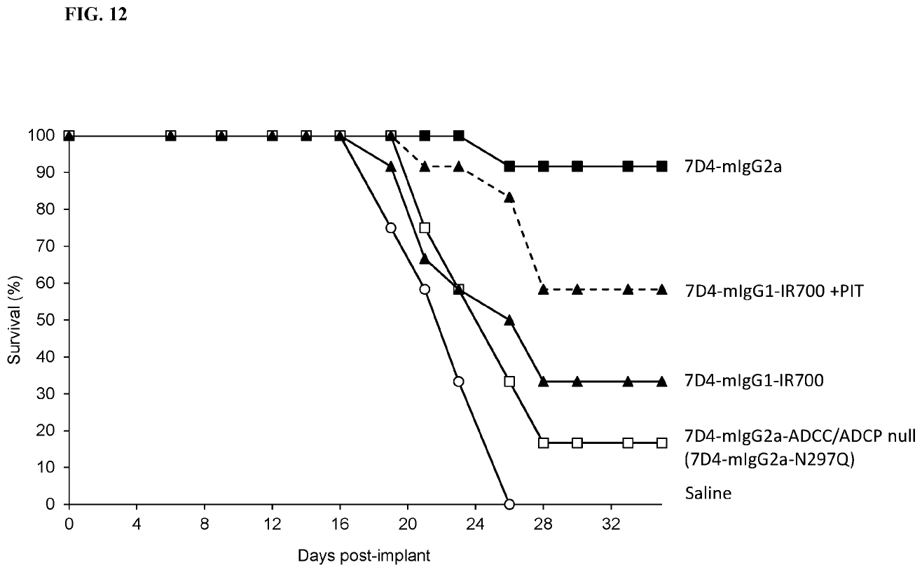

As shown in FIG. 12, the survival curves corroborated the tumor growth inhibition observed. Consistent with the results of tumor growth inhibition, mice administered IL-2 non-blocking anti-CD25 mIgG2a antibody, with increased ADCC activity, exhibited the greatest survival (7D4-mIgG2a; filled squares), followed by IL-2 non-blocking anti-CD25 PIT (7D4-mIgG1-IR700 PIT; filled triangles, dashed line), IL-2 non-blocking anti-CD25 conjugate alone (no illumination) (7D4-mIgG1-IR700; filled triangles, solid line), IL-2 blocking anti-CD25-IgG2a-ADCC/ADCP null antibody (7D4-mIgG2a-N297Q; open squares), and saline control (saline; open circles).

These results indicate that IL-2 non-blocking anti-CD25 PIT can effectively suppress tumor growth even when ADCC/ADCP activity is limited. Therefore, unlike the unconjugated IL-2 non-blocking anti-CD25 antibody, ADCC/ADCP activity is not required for IL-2 non-blocking anti-CD25 PIT efficacy.

### Example 7: Effects of IL-2 Non-Blocking Anti-CD25 Antibody and IL-2 Non-Blocking Anti-CD25 Photoimmunotherapy (PIT) On Cold Tumors With and Without Anti-PD-1 Antibody Treatment

This example describes the effects of naked (unconjugated) IL-2 non-blocking anti-CD25 antibody and IL-2 non-blocking anti-CD25 PIT on immune “cold” tumors, i.e. tumors characterized by low immunoresponsiveness (low levels of and exhausted tumor-infiltrating lymphocytes (TILs), insufficient tumor antigen burden, and immunosuppressive microenvironment). Cold tumors characteristically do not respond well to immune checkpoint inhibitor therapies.

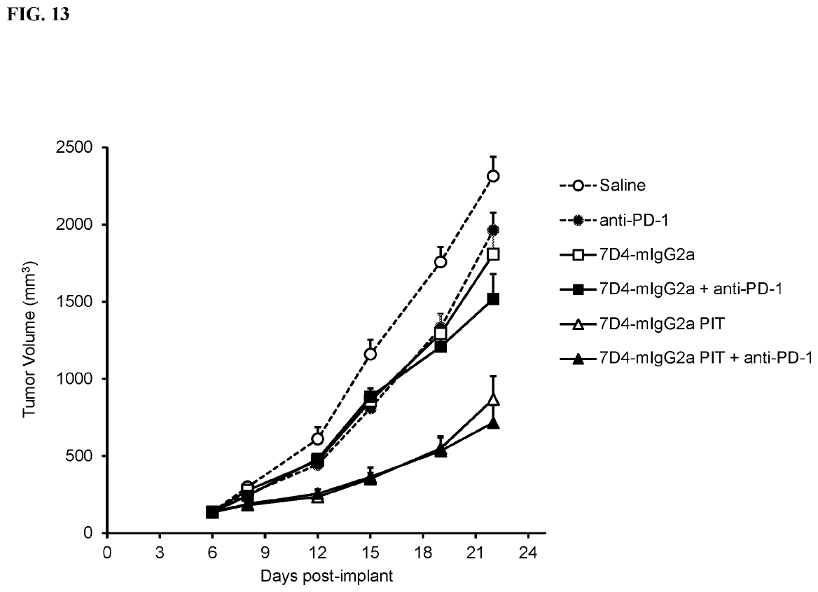

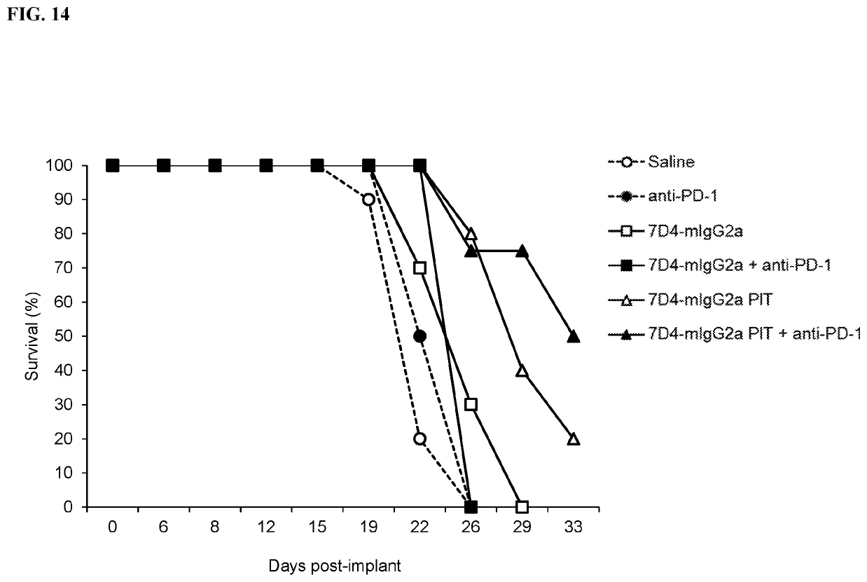

BALB/c mice (16-18 g) were inoculated with 1×10⁵ 4T1 cells per mouse subcutaneously on the right hind flank. When allograft tumors grew to a volume of about 140 mm³ (6 days after tumor cell inoculation), mice were administered saline (100 μL by intraperitoneal (IP) injection), 7D4-mIgG2a (200 μg by IP injection), or 7D4-mIgG2a-IR700 (100 μg by retroorbital injection). Half of each treatment group were also treated with anti-PD-1 antibody RMP1-14 (10 mg/kg) 3×/week (on Days 6, 9, 13, etc). Twenty-four hours after administering the conjugate, tumors in the group administered 7D4-mIgG2a-IR700 were illuminated at 690 nm at a dosage of 150 J/cm² (7D4-mIgG2a PIT). Tumor growth (FIG. 13) and survival (FIG. 14) were measured over time.

7D4-mIgG2a PIT, alone (open triangles) or in combination with anti-PD-1 (closed triangles), substantially inhibited the growth of the “cold” tumors compared to saline (open circles) or anti-PD-1 monotherapy (closed circles) (FIG. 13). 7D4-mIgG2a antibody, alone (open squares) or in combination with anti-PD-1 (closed squares), did not substantially inhibit effect on the growth of the “cold” tumors (FIG. 13). Survival curves were consistent with the anti-tumor effects of each treatment (FIG. 14). These data support the finding that IL-2 non-blocking anti-CD25 PIT effectively inhibited cold tumor growth while IL-2 non-blocking antibody alone had minimal effects on cold tumor growth with or without PD-1 treatment.

The present invention is not to be limited in scope by the embodiments disclosed herein, which are intended as single illustrations of individual aspects of the invention, and any that are functionally equivalent are within the scope of the invention. Various modifications to the compositions and methods of the invention, in addition to those described herein, will become apparent to those skilled in the art from the foregoing description and teachings, and are similarly intended to fall within the scope of the invention. Such modifications or other embodiments can be practiced without departing from the true scope and spirit of the invention.

SEQUENCES

| SEQ ID | | |
|---|---|---|
| NO. | Sequence | Description |
| 1 | EVQLVESGGDLVQPRGSLKLSCAASGFTFSSYGMSWVRQTPDKRLE | 7G7B6 V_{H} |
| | LVATINGYGDTTYYPDSVKGRFTISRDNAKNTLYLQMSSLKSEDTA | |
| | MYFCARDRDYGNSYYYALDYWGQGTSVTVSS | |
| 2 | QIVLSQSPAILSASPGERVTMTCRASSSVSFMHWLQQKPGSSPKPW | 7G7B6 V_{L} |
| | IYATSNLASGVSARFSGSGSGTSYSLTITRVEAEDAATYYCQQWSS | |
| | NPPAFGGGTKLEIK | |
| 3 | QVQLKESGPGLVAPSQSLSITCTVSGFSLTSYGIQWVRQPPGKGLE | MA251 V_{H} |
| | WLGVIWAGGSTNYNSALMSRLSISKDNSKSQVFLKMNSLQTDDTAM | |
| | YYCARAYGYDGSWLAYWGQGTLVTVSS | |
| 4 | QIVLSQSPAILSASPGEKVTMTCRASSSVSYMHWYQQKPGSSPKPW | MA251 V_{L} |
| | IFATSNLASGVPARFSGSGSGTSYSLTINRVEAEDADTYYCQQWSS | |
| | NPPTFGGGTKLEIK | |
| 5 | EVQLVESGGGLIQPGGSLRLSCAASGFTLDSYGVSWVRQAPGKGLE | Clone A V_{H} |
| | WVGVTSSGGSAYYADSVKGRFTISRDNSKNTLYLQMNSLRAEDTAV | |
| | YYCARDRYVYTGGYLYHYGMDLWGQGTLVTVSS | |
| 6 | DIQMTQSPSSLSASVGDRVTITCRASQSISDYLAWYQQKPGKVPKL | Clone A V_{L} |
| | LIYAASTLPFGVPSRFSGSGSGTDFTLTISSLQPEDVATYYCQGTY | |
| | DSSDWYWAFGGGTKVEI | |
| 7 | EVQLLESGGGLVQPGGSLRLSCAASGFSVDIYDMSWVRQAPGKGLE | Clone B V_{H} |
| | WVAYISSSLGATYYADSVKGRFTISRDNSKNTLYLQMNSLRAEDTA | |
| | VYYCARERIYSVYTLDYYAMDLWGQGTLVTVSS | |
| 8 | DIQMTQSPSSLSASVGDRVTITCQASQGITNNLNWYQQKPGKVPKL | Clone B V_{L} |
| | LIYAASTLQSGVPSRFSGSGSGTDFTLTISSLQPEDVATYYCQQGY | |
| | TTSNVDNAFGGGTKVEIK | |
| 9 | EVQLLESGGGLVQPGGSLRLSCAASGFTFSSYGMSWVRQAPGKGLE | Clone C, D V_{H} |
| | LVSTINGYGDTTYYPDSVKGRFTISRDNSKNTLYLQMNSLRAEDTA | |
| | VYYCARDRDYGNSYYYALDYWGQGTLVTVSS | |
| 10 | EVQLLESGGGLVQPGGSLRLSCAASGFTFSSYGMSWVRQAPGKGLE | Clone E, F V_{H} |
| | LVSTINGYGDTTYYPDSVKGRFTISRDNAKNTLYLQMNSLRAEDTA | |
| | VYFCARDRDYGNSYYYALDYWGQGTLVTVSS | |
| 11 | EIVLTQSPGTLSLSPGERATLSCRASSSVSFMHWLQQKPGQAPRPL | Clone C, E V_{L} |
| | IYATSNLASGIPDRFSGSGSGTDYTLTISRLEPEDFAVYYCQQWSS | |
| | NPPAFGQGTKLEIK | |
| 12 | QIVLTQSPGTLSLSPGERATLSCRASSSVSFMHWLQQKPGQSPRPL | Clone D, F V_{L} |
| | IYATSNLASGIPDRFSGSGSGTDYTLTISRLEPEDFAVYYCQQWSS | |
| | NPPAFGQGTKLEIK | |
| 13 | QVQLQESGPGLVKPSETLSLTCTVSGFSLTSYGIQWIRQPPGKGLE | Clone G, H, I, J V_{H} |
| | WIGVIWAGGSTNYNSALMSRVTISKDNSKNQFSLKLSSVTAADTAV | |
| | YYCARAYGYDGSWLAYWGQGTLVTVSS | |
| 14 | QVQLQESGPGLVKPSETLSLTCTVSGFSLTSYGIQWVRQPPGKGLE | Clone K, L, M, N V_{H} |
| | WIGVIWAGGSTNYNSALMSRVTISKDNSKSQFSLKLSSVTAADTAV | |
| | YYCARAYGYDGSWLAYWGQGTLVTVSS | |
| 15 | QVQLVESGGGVVQPGGSLRLSCAVSGFSLTSYGIQWVRQAPGKGLE | Clone O, P, Q, R V_{H} |
| | WVSVIWAGGSTNYNSALMSRFTISKDNSKSTLYLQMNSLRAEDTAV | |
| | YYCARAYGYDGSWLAYWGQGTLVTVSS | |
| 16 | EIVLTQSPATLSLSPGERATLSCRASSSVSYMHWYQQKPGQAPRPL | Clone G, K, O V_{L} |
| | IFATSNLASGIPARFSGSGSGTDFTLTISSLEPEDFAVYYCQQWSS | |
| | NPPTFGGGTKLEIK | |
| 17 | QIVLTQSPATLSLSPGERATLSCRASSSVSYMHWYQQKPGQAPRPL | Clone H, L, P V_{L} |
| | IFATSNLASGIPARFSGSGSGTDYTLTISSLEPEDFAVYYCQQWSS | |
| | NPPTFGGGTKLEIK | |
| 18 | DIQMTQSPSSLSASVGDRVTITCRASSSVSYMHWYQQKPGKAPKPL | Clone I, M, Q V_{L} |
| | IFATSNLASGVPSRFSGSGSGTDYTLTISSLQPEDFATYYCQQWSS | |
| | NPPTFGGGTKLEIK | |
| 19 | QIQLTQSPSSLSASVGDRVTITCRASSSVSYMHWYQQKPGKSPKPL | Clone J, N, R V_{L} |
| | IFATSNLASGVPSRFSGSGSGTDYTLTISSLQPEDFATYYCQQWSS | |
| | NPPTFGGGTKLEIK | |
| 20 | GFTFSSY | 7G7B6, Clone C, D, |
| | | E, F CDRH1 |
| 21 | NGYGDT | 7G7B6, Clone C, D, |
| | | E, F CDRH2 |
| 22 | DRDYGNSYYYALDY | 7G7B6, Clone C, D, |
| | | E, F CDRH3 |
| 23 | RASSSVSFMH | 7G7B6, Clone C, E, |
| | | D, F CDRL1 |
| 24 | ATSNLAS | 7G7B6, MA251, |
| | | Clone C, E, D, F, G, |
| | | K, O, H, L, P, I, M, |
| | | Q, J, N, R CDRL2 |
| 25 | QQWSSNPPA | 7G7B6, Clone C, E, |
| | | D, F CDRL3 |
| 26 | GFSLTSY | MA251, Clone G, H, |
| | | I, J, K, L, M, N, O, P, |
| | | Q, R CDRH1 |
| 27 | WAGGS | MA251, Clone G, H, |
| | | I, J, K, L, M, N, O, P, |
| | | Q, R CDRH2 |
| 28 | AYGYDGSWLAY | MA251, Clone G, H, |
| | | I, J, K, L, M, N, O, P, |
| | | Q, R CDRH3 |
| 29 | RASSSVSYMH | MA251, Clone G, K, |
| | | O, H, L, P, I, M, Q, J, |
| | | N, R CDRL1 |
| 30 | QQWSSNPPT | MA251, Clone G, K, |
| | | O, H, L, P, I, M, Q, J, |
| | | N, R CDRL3 |
| 31 | GFTLDSY | Clone A CDRH1 |
| 32 | SSGGS | Clone A CDRH2 |
| 33 | DRYVYTGGYLYHYGMDL | Clone A CDRH3 |
| 34 | RASQSISDYLA | Clone A CDRL1 |
| 35 | AASTLPF | Clone A CDRL2 |
| 36 | QGTYDSSDWYWA | Clone A CDRL3 |
| 37 | GFSVDIY | Clone B CDRH1 |
| 38 | SSSLGA | Clone B CDRH2 |
| 39 | ERIYSVYTLDYYAMDL | Clone B CDRH3 |
| 40 | QASQGITNNLN | Clone B CDRL1 |
| 41 | AASTLQS | Clone B CDRL2 |
| 42 | QQGYTTSNVDNA | Clone B CDRL3 |
| 43 | EVQLQQSGAALVKPGASVKMSCKASGYSFPDSWVTWVKQSHGKSLE | 7D4 V_{H} |
| | WIGDIFPNSGATNFNEKFKGKATLTVDKSTSTAYMELSRLTSEDSA | |
| | IYYCTRLDYGYWGQGVMVTVSS | |
| 44 | AKTTPPSVYPLAPGSAAQTNSMVTLGCLVKGYFPEPVTVTWNSGSL | 7D4-mIgG1 CH |
| | SSGVHTFPAVLQSDLYTLSSSVTVPSSTWPSQTVTCNVAHPASSTK | |
| | VDKKIVPRDCGCKPCICTVPEVSSVFIFPPKPKDVLMISLTPKVTC | |
| | VVVDISKDDPEVQFSWFVDDVEVHTAQTKPREEQINSTFRSVSELP | |
| | ILHQDWLNGKEFKCRVNSAAFPAPIEKTISKTKGRPKAPQVYTIPP | |
| | PKEQMAKDKVSLTCMITNFFPEDITVEWQWNGQPAENYKNTQPIMD | |
| | TDGSYFVYSKLNVQKSNWEAGNTFTCSVLHEGLHNHHTEKSLSHSP | |
| | GK | |
| 45 | AKTTAPSVYPLAPVCGDTTGSSVTLGCLVKGYFPEPVTLTWNSGSL | 7D4-mIgG2a CH |
| | SSGVHTFPAVLQSDLYTLSSSVTVTSSTWPSQSITCNVAHPASSTK | |
| | VDKKIEPRGPTIKPCPPCKCPAPNLLGGPSVFIFPPKIKDVLMISL | |
| | SPIVTCVVVDVSEDDPDVQISWFVNNVEVHTAQTQTHREDYNSTLR | |
| | VVSALPIQHQDWMSGKEFKCKVNNKDLPAPIERTISKPKGSVRAPQ | |
| | VYVLPPPEEEMTKKQVTLTCMVTDFMPEDIYVEWINNGKTELNYKN | |
| | TEPVLDSDGSYFMYSKLRVEKKNWVERNSYSCSVVHEGLHNHHTTK | |
| | SFSRTPGK | |
| 46 | DVVLTQTPPTLSATIGQSVSISCRSSQSLLHSNGNTYLNWLLQRPG | 7D4 V_{L} |
| | QPPQLLIYLASRLESGVPNRFSGSGSGTDFTLKISGVEAEDLGVYY | |
| | CVQSSHFPNTFGVGTKLEIK | |
| 47 | RADAAPTVSIFPPSSEQLTSGGASVVCFLNNFYPKDINVKWKIDGS | 7D4-mIgG1 CL, |
| | ERQNGVLNSWTDQDSKDSTYSMSSTLTLTKDEYERHNSYTCEATHK | 7D4-mIgG2a CL |
| | TSTSPIVKSFNRNEC | |
| 48 | EVQLQQSGAELVRPGTSVKLSCKVSGDTITAYYIHFVKQRPGQGLE | PC61 V_{H} |
| | WIGRIDPEDDSTEYAEKFKNKATITANTSSNTAHLKYSRLTSEDTA | |
| | TYFCTTDNMGATEFVYWGQGTLVTVSS | |
| 49 | AKTTPPSVYPLAPGSAAQTNSMVTLGCLVKGYFPEPVTVTWNSGSL | PC61-mIgG1 CH |
| | SSGVHTFPAVLESDLYTLSSSVTVPSSPRPSETVTCNVAHPASSTK | |
| | VDKKIVPRDCGCKPCICTVPEVSSVFIFPPKPKDVLTITLTPKVTC | |
| | VVVDISKDDPEVQFSWFVDDVEVHTAQTQPREEQFNSTFRSVSELP | |
| | IMHQDWLNGKEFKCRVNSAAFPAPIEKTISKTKGRPKAPQVYTIPP | |
| | PKEQMAKDKVSLTCMITDFFPEDITVEWQWNGQPAENYKNTQPIMN | |
| | TNGSYFVYSKLNVQKSNWEAGNTFTCSVLHEGLHNHHTEKSLSHSP | |
| 50 | QVVLTQPKSVSASLESTVKLSCKLNSGNIGSYYMHWYQQREGRSPT | PC61 V_{L} |
| | NLIYRDDKRPDGAPDRFSGSIDISSNSAFLTINNVQTEDEAMYFCH | |
| | SYDGRMYIFGGGTKLTVL | |
| 51 | GQPKSSPSVTLFPPSSEELETNKATLVCTITDFYPGVVTVDWKVDG | PC61-mIgG1 CL |
| | TPVTQGMETTQPSKQSNNKYMASSYLTLTARAWERHSSYSCQVTHE | |
| | GHTVEKSLSRADCS | |

1. A conjugate comprising an antibody or antigen-binding fragment that specifically binds a CD25 without substantially blocking or interfering with IL-2 signaling, and a Si-phthalocyanine dye; wherein the conjugate is activated by illumination at a wavelength between at or about 600 nm and at or about 850 nm to effect cell killing.

2. The conjugate of claim 1, wherein the activated conjugate does not substantially block or interfere with IL-2 signaling.

3. The conjugate of claim 1 or 2, wherein the Si-phthalocyanine dye is IR700.

4. The conjugate of claim 1 or 2, wherein the Si-phthalocyanine dye has the structure of Formula (I): 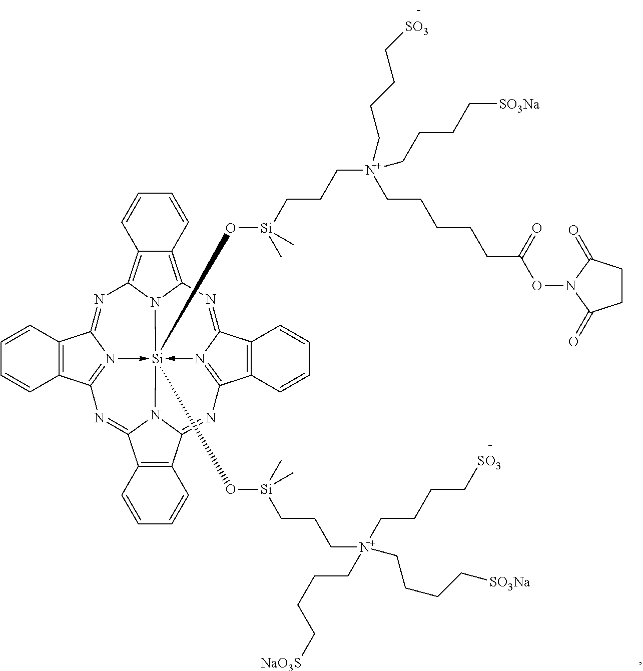
or is a salt, stereoisomer, or tautomer thereof.

5. The conjugate of any of claims 1-4, wherein the activated conjugate effects tumor inhibition or killing at a higher level, activity or potency than the unconjugated antibody.

6. The conjugate of any of claims 1-5, wherein the antibody or antigen-binding fragment comprises a heavy chain variable (V_{H}) region and a light chain variable (V_{L}) region, wherein:
the V_{H} region comprises a heavy chain complementarity determining region 1 (CDR-H1), a heavy chain complementarity determining region 2 (CDR-H2) and a heavy chain complementarity determining region 3 (CDR-H3) contained within the V_{H} region amino acid sequence set forth in SEQ ID NO: 1 and the V_{L} region comprises a light chain complementarity determining region 1 (CDR-L1), a light chain complementarity determining region 2 (CDR-L2) and a light chain complementarity determining region 3 (CDR-L3) contained within the V_{L} region amino acid sequence set forth in SEQ ID NO: 2;
the V_{H} region comprises a CDR-H1, a CDR-H2 and a CDR-H3 contained within the V_{H} region amino acid sequence set forth in SEQ ID NO: 3 and the V_{L} region comprises a CDR-L1, a CDR-L2 and a CDR-L3 contained within the V_{L} region amino acid sequence set forth in SEQ ID NO: 4;
the V_{H} region comprises a CDR-H1, a CDR-H2 and a CDR-H3 contained within the V_{H} region amino acid sequence set forth in SEQ ID NO: 5 and the V_{L} region comprises a CDR-L1, a CDR-L2 and a CDR-L3 contained within the V_{L} region amino acid sequence set forth in SEQ ID NO: 6;
the V_{H} region comprises a CDR-H1, a CDR-H2 and a CDR-H3 contained within the V_{H} region amino acid sequence set forth in SEQ ID NO: 7 and the V_{L} region comprises a CDR-L1, a CDR-L2 and a CDR-L3 contained within the V_{L} region amino acid sequence set forth in SEQ ID NO: 8;
the V_{H} region comprises a CDR-H1, a CDR-H2 and a CDR-H3 contained within the V_{H} region amino acid sequence set forth in SEQ ID NO: 9 and the V_{L} region comprises a CDR-L1, a CDR-L2 and a CDR-L3 contained within the V_{L} region amino acid sequence set forth in SEQ ID NO: 11;
the V_{H} region comprises a CDR-H1, a CDR-H2 and a CDR-H3 contained within the V_{H} region amino acid sequence set forth in SEQ ID NO: 9 and the V_{L} region comprises a CDR-L1, a CDR-L2 and a CDR-L3 contained within the V_{L} region amino acid sequence set forth in SEQ ID NO: 12;
the V_{H} region comprises a CDR-H1, a CDR-H2 and a CDR-H3 contained within the V_{H} region amino acid sequence set forth in SEQ ID NO: 10 and the V_{L} region comprises a CDR-L1, a CDR-L2 and a CDR-L3 contained within the V_{L} region amino acid sequence set forth in SEQ ID NO: 11;
the V_{H} region comprises a CDR-H1, a CDR-H2 and a CDR-H3 contained within the V_{H} region amino acid sequence set forth in SEQ ID NO: 10 and the V_{L} region comprises a CDR-L1, a CDR-L2 and a CDR-L3 contained within the V_{L} region amino acid sequence set forth in SEQ ID NO: 12;
the V_{H} region comprises a CDR-H1, a CDR-H2 and a CDR-H3 contained within the V_{H} region amino acid sequence set forth in SEQ ID NO: 13 and the V_{L} region comprises a CDR-L1, a CDR-L2 and a CDR-L3 contained within the V_{L} region amino acid sequence set forth in SEQ ID NO: 16;
the V_{H} region comprises a CDR-H1, a CDR-H2 and a CDR-H3 contained within the V_{H} region amino acid sequence set forth in SEQ ID NO: 13 and the V_{L} region comprises a CDR-L1, a CDR-L2 and a CDR-L3 contained within the V_{L} region amino acid sequence set forth in SEQ ID NO: 17;
the V_{H} region comprises a CDR-H1, a CDR-H2 and a CDR-H3 contained within the V_{H} region amino acid sequence set forth in SEQ ID NO: 13 and the V_{L} region comprises a CDR-L1, a CDR-L2 and a CDR-L3 contained within the V_{L} region amino acid sequence set forth in SEQ ID NO: 18;
the V_{H} region comprises a CDR-H1, a CDR-H2 and a CDR-H3 contained within the V_{H} region amino acid sequence set forth in SEQ ID NO: 13 and the V_{L} region comprises a CDR-L1, a CDR-L2 and a CDR-L3 contained within the V_{L} region amino acid sequence set forth in SEQ ID NO: 19;
the V_{H} region comprises a CDR-H1, a CDR-H2 and a CDR-H3 contained within the V_{H} region amino acid sequence set forth in SEQ ID NO: 14 and the V_{L} region comprises a CDR-L1, a CDR-L2 and a CDR-L3 contained within the V_{L} region amino acid sequence set forth in SEQ ID NO: 16;
the V_{H} region comprises a CDR-H1, a CDR-H2 and a CDR-H3 contained within the V_{H} region amino acid sequence set forth in SEQ ID NO: 14 and the V_{L} region comprises a CDR-L1, a CDR-L2 and a CDR-L3 contained within the V_{L} region amino acid sequence set forth in SEQ ID NO: 17;
the V_{H} region comprises a CDR-H1, a CDR-H2 and a CDR-H3 contained within the V_{H} region amino acid sequence set forth in SEQ ID NO: 14 and the V_{L} region comprises a CDR-L1, a CDR-L2 and a CDR-L3 contained within the V_{L} region amino acid sequence set forth in SEQ ID NO: 18;
the V_{H} region comprises a CDR-H1, a CDR-H2 and a CDR-H3 contained within the V_{H} region amino acid sequence set forth in SEQ ID NO: 14 and the V_{L} region comprises a CDR-L1, a CDR-L2 and a CDR-L3 contained within the V_{L} region amino acid sequence set forth in SEQ ID NO: 19;
the V_{H} region comprises a CDR-H1, a CDR-H2 and a CDR-H3 contained within the V_{H} region amino acid sequence set forth in SEQ ID NO: 15 and the V_{L} region comprises a CDR-L1, a CDR-L2 and a CDR-L3 contained within the V_{L} region amino acid sequence set forth in SEQ ID NO: 16;
the V_{H} region comprises a CDR-H1, a CDR-H2 and a CDR-H3 contained within the V_{H} region amino acid sequence set forth in SEQ ID NO: 15 and the V_{L} region comprises a CDR-L1, a CDR-L2 and a CDR-L3 contained within the V_{L} region amino acid sequence set forth in SEQ ID NO: 17;
the V_{H} region comprises a CDR-H1, a CDR-H2 and a CDR-H3 contained within the V_{H} region amino acid sequence set forth in SEQ ID NO: 15 and the V_{L} region comprises a CDR-L1, a CDR-L2 and a CDR-L3 contained within the V_{L} region amino acid sequence set forth in SEQ ID NO: 18; or
the V_{H} region comprises a CDR-H1, a CDR-H2 and a CDR-H3 contained within the V_{H} region amino acid sequence set forth in SEQ ID NO: 15 and the V_{L} region comprises a CDR-L1, a CDR-L2 and a CDR-L3 contained within the V_{L} region amino acid sequence set forth in SEQ ID NO: 19.

7. The conjugate of any of claims 1-6, wherein the antibody or antigen-binding fragment comprises a heavy chain variable (V_{H}) region and a light chain variable (V_{L}) region, wherein:
the V_{H} region comprises a heavy chain complementarity determining region 1 (CDR-H1) comprising the amino acid sequence set forth in SEQ ID NO: 20; a heavy chain complementarity determining region 2 (CDR-H2) comprising the amino acid sequence set forth in SEQ ID NO: 21, and a heavy chain complementarity determining region 3 (CDR-H3) comprising the amino acid sequence set forth in SEQ ID NO: 22, and the V_{L} region comprises a light chain complementarity determining region 1 (CDR-L1) comprising the amino acid sequence set forth in SEQ ID NO: 23; a light chain complementarity determining region 2 (CDR-L2) comprising the amino acid sequence set forth in SEQ ID NO: 24, and a light chain complementarity determining region 3 (CDR-L3) comprising the amino acid sequence set forth in SEQ ID NO: 25;
the V_{H} region comprises a CDR-H1 comprising the amino acid sequence set forth in SEQ ID NO: 26; a CDR-H2 comprising the amino acid sequence set forth in SEQ ID NO: 27, and a CDR-H3 comprising the amino acid sequence set forth in SEQ ID NO: 28, and the V_{L} region comprises a CDR-L1 comprising the amino acid sequence set forth in SEQ ID NO: 29; a CDR-L2 comprising the amino acid sequence set forth in SEQ ID NO: 24, and a CDR-L3 comprising the amino acid sequence set forth in SEQ ID NO: 30;
the V_{H} region comprises a CDR-H1 comprising the amino acid sequence set forth in SEQ ID NO: 31; a CDR-H2 comprising the amino acid sequence set forth in SEQ ID NO: 32, and a CDR-H3 comprising the amino acid sequence set forth in SEQ ID NO: 33, and the V_{L} region comprises a CDR-L1 comprising the amino acid sequence set forth in SEQ ID NO: 34; a CDR-L2 comprising the amino acid sequence set forth in SEQ ID NO: 35, and a CDR-L3 comprising the amino acid sequence set forth in SEQ ID NO: 36; or
the V_{H} region comprises a CDR-H1 comprising the amino acid sequence set forth in SEQ ID NO: 37; a CDR-H2 comprising the amino acid sequence set forth in SEQ ID NO: 38, and a CDR-H3 comprising the amino acid sequence set forth in SEQ ID NO: 39, and the V_{L} region comprises a CDR-L1 comprising the amino acid sequence set forth in SEQ ID NO: 40; a CDR-L2 comprising the amino acid sequence set forth in SEQ ID NO: 41, and a CDR-L3 comprising the amino acid sequence set forth in SEQ ID NO: 42.

8. The conjugate of any of claims 1-7, wherein the antibody or antigen-binding fragment comprises a heavy chain variable (V_{H}) region and a light chain variable (V_{L}) region, wherein:
the V_{H} region comprises the amino acid sequence set forth in SEQ ID NO: 1 and the V_{L} region comprises the amino acid sequence set forth in SEQ ID NO: 2;
the V_{H} region comprises the amino acid sequence set forth in SEQ ID NO: 3 and the V_{L} region comprises the amino acid sequence set forth in SEQ ID NO: 4;
the V_{H} region comprises the amino acid sequence set forth in SEQ ID NO: 5 and the V_{L} region comprises the amino acid sequence set forth in SEQ ID NO: 6;
the V_{H} region comprises the amino acid sequence set forth in SEQ ID NO: 7 and the V_{L} region comprises the amino acid sequence set forth in SEQ ID NO: 8;
the V_{H} region comprises the amino acid sequence set forth in SEQ ID NO: 9 and the V_{L} region comprises the amino acid sequence set forth in SEQ ID NO: 11;
the V_{H} region comprises the amino acid sequence set forth in SEQ ID NO: 9 and the V_{L} region comprises the amino acid sequence set forth in SEQ ID NO: 12;
the V_{H} region comprises the amino acid sequence set forth in SEQ ID NO: 10 and the V_{L} region comprises the amino acid sequence set forth in SEQ ID NO: 11;
the V_{H} region comprises the amino acid sequence set forth in SEQ ID NO: 10 and the V_{L} region comprises the amino acid sequence set forth in SEQ ID NO: 12;
the V_{H} region comprises the amino acid sequence set forth in SEQ ID NO: 13 and the V_{L} region comprises the amino acid sequence set forth in SEQ ID NO: 16;
the V_{H} region comprises the amino acid sequence set forth in SEQ ID NO: 13 and the V_{L} region comprises the amino acid sequence set forth in SEQ ID NO: 17;
the V_{H} region comprises the amino acid sequence set forth in SEQ ID NO: 13 and the V_{L} region comprises the amino acid sequence set forth in SEQ ID NO: 18;
the V_{H} region comprises the amino acid sequence set forth in SEQ ID NO: 13 and the V_{L} region comprises the amino acid sequence set forth in SEQ ID NO: 19;
the V_{H} region comprises the amino acid sequence set forth in SEQ ID NO: 14 and the V_{L} region comprises the amino acid sequence set forth in SEQ ID NO: 16;
the V_{H} region comprises the amino acid sequence set forth in SEQ ID NO: 14 and the V_{L} region comprises the amino acid sequence set forth in SEQ ID NO: 17;
the V_{H} region comprises the amino acid sequence set forth in SEQ ID NO: 14 and the V_{L} region comprises the amino acid sequence set forth in SEQ ID NO: 18;
the V_{H} region comprises the amino acid sequence set forth in SEQ ID NO: 14 and the V_{L} region comprises the amino acid sequence set forth in SEQ ID NO: 19;
the V_{H} region comprises the amino acid sequence set forth in SEQ ID NO: 15 and the V_{L} region comprises the amino acid sequence set forth in SEQ ID NO: 16;
the V_{H} region comprises the amino acid sequence set forth in SEQ ID NO: 15 and the V_{L} region comprises the amino acid sequence set forth in SEQ ID NO: 17;
the V_{H} region comprises the amino acid sequence set forth in SEQ ID NO: 15 and the V_{L} region comprises the amino acid sequence set forth in SEQ ID NO: 18; or
the V_{H} region comprises the amino acid sequence set forth in SEQ ID NO: 15 and the V_{L} region comprises the amino acid sequence set forth in SEQ ID NO: 19.

9. The conjugate of any of claims 1-8, wherein the antibody or antigen-binding fragment comprises MA251, 7G7B6 or an antigen-binding portion thereof.

10. The conjugate of any of claims 1-9, wherein the conjugate exhibits one or more Fc-mediated effector function(s).

11. The conjugate of any of claims 1-9, wherein the conjugate lacks Fc-mediated effector function(s), exhibits substantially reduced Fc-mediated effector function(s) or does not exhibit substantial Fc-mediated effector function(s).

12. The conjugate of claim 11, wherein the activated conjugate is capable of cell killing in the absence of substantial Fc-mediated effector function(s).

13. The conjugate of any one of claims 10-12, wherein the Fc-mediated effector function is selected from one or more of an antibody-dependent cellular cytotoxicity (ADCC), antibody-dependent cellular phagocytosis (ADCP) or complement-dependent cytotoxicity (CDC).

14. The conjugate of any of claims 1-13, wherein the conjugate, when activated has at least two modes of action to effect cell killing.

15. The conjugate of claim 14, wherein one of the at least two modes of action is ADCC independent.

16. The conjugate of claim 1-15, wherein the activated conjugate exhibits at least one mode of cell killing or tumor inhibition not present in the unconjugated antibody.

17. The conjugate of any of claims 1-16, wherein the conjugate comprises an IgG1 Fc region or an IgG1 isotype, an IgG2 Fc region or an IgG2 isotype, IgG3 Fc region or an IgG3 isotype, or an IgG4 Fc region or an IgG4 isotype.

18. The conjugate of any of claims 1-17, wherein the antibody or antibody-binding fragment comprises an IgG1 Fc region or an IgG1 isotype.

19. The conjugate of claim 18, wherein the IgG1 Fc region does not exhibit enhanced antibody-dependent cellular cytotoxicity (ADCC) effector function.

20. The conjugate of any of claims 1-17, wherein the antibody or antibody-binding fragment comprises an IgG2 Fc region or an IgG2 isotype.

21. The conjugate of claim 20, wherein the IgG2 Fc region comprises a substitution that reduces or abrogates ADCC effector function.

22. The conjugate of claim 21, wherein the substitution is a substitution of glutamine for the asparagine in the Fc region at the position corresponding to 297 according to EU numbering (N297Q).

23. The conjugate of any of claims 1-22, wherein the antibody or antigen-binding fragment is a human, chimeric, or humanized antibody or antigen-binding fragment.

24. The conjugate of any of claims 1-23, wherein the antibody or antigen binding fragment comprises an Fc region of a human immunoglobulin and/or human antibody framework regions.

25. A method of treating a tumor or a lesion in a subject, comprising:
a) administering to the subject the conjugate of any of claims 1-24; and
b) illuminating a target site within the subject with a wavelength of between at or about 600 nm and at or about 850 nm, and at a dose of from at or about 25 J/cm² to at or about 400 J/cm² or from at or about 2 J/cm fiber length to at or about 500 J/cm fiber length, thereby activating the conjugate;
whereby the growth, volume or dimensions of the tumor or the lesion is reduced or inhibited.

26. A method of treating a tumor or a lesion in a subject, comprising:
a) administering to the subject a conjugate, wherein the conjugate comprises an antibody or antigen-binding fragment that specifically binds a CD25 without substantially blocking or interfering with IL-2 signaling, and a Si-phthalocyanine dye; and
b) illuminating a target site within the subject with a wavelength of between at or about 600 nm and at or about 850 nm, and at a dose of from at or about 25 J/cm² to at or about 400 J/cm² or from at or about 2 J/cm fiber length to at or about 500 J/cm fiber length, thereby activating the conjugate;
whereby the growth, volume or dimensions of the tumor or the lesion is reduced or inhibited.

27. The method of claim 25 or 26, wherein the tumor or lesion to be treated, or the tumor microenvironment (TME) of the tumor or lesion to be treated, contains reduced levels of immune effector cells.

28. The method of claim 26 or 27, wherein the Si-phthalocyanine dye is IR700 and the illuminating step is performed at a wavelength of 690 nm±20 nm.

29. The method of claim 26 or 27, wherein the Si-phthalocyanine dye comprises Formula (I) 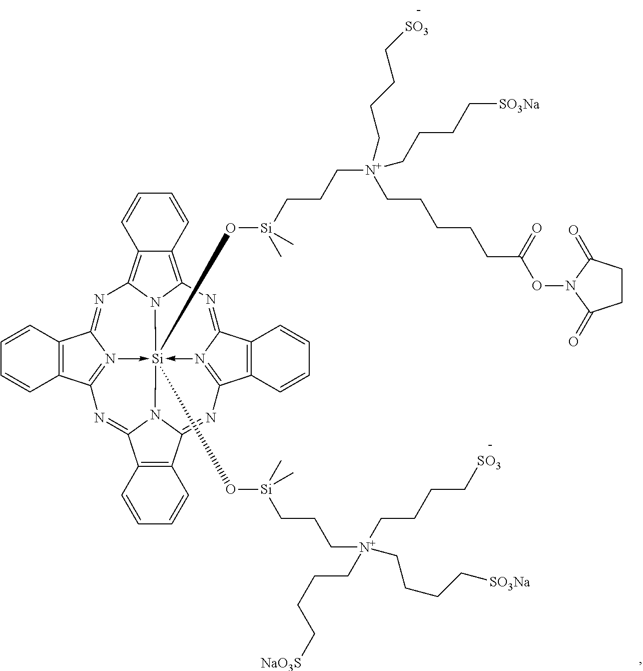
or a salt, stereoisomer, or tautomer thereof, and the illuminating step is performed at a wavelength of 660 nm±50 nm.

30. The method of any of claims 27-29, wherein the immune effector cells are selected from one or more of macrophages, natural killer (NK) cells, neutrophils, and eosinophils.

31. The method of any of claims 25-30, wherein the target site is illuminated within about 24±4 hours after administering the conjugate.

32. The method of any of claims 25-31, wherein the target site is illuminated at an optical power of between at or about 50 mW/cm² and at or about 200 mW/cm².

33. The method of any of claims 25-31, wherein the target site is illuminated at an optical power of between at or about 100 mW/cm fiber length and at or about 500 mW/cm fiber length.

34. The method of any of claims 25-33, wherein the target site is illuminated for between at or about 120 seconds and at or about 600 seconds.

35. The method of any of claims 25-34, wherein the tumor or lesion is resistant or non-responsive to immune checkpoint inhibitor therapy.

36. The method of any of claims 25-34, wherein the tumor or lesion has a reduced response or is non-responsive to the unconjugated antibody.

37. The method of any of claims 26-36, wherein the conjugate exhibits one or more Fc-mediated effector function(s).

38. The method of any of claims 26-36, wherein the conjugate lacks Fc-mediated effector function(s), exhibits substantially reduced Fc-mediated effector function(s) or does not exhibit substantial Fc-mediated effector function(s).

39. The method of claim 38, wherein the activated conjugate is capable of cell killing in the absence of substantial Fc-mediated effector function(s).

40. The method of any of claims 37-39, wherein the Fc-mediated effector function is selected from one or more of an antibody-dependent cellular cytotoxicity (ADCC), antibody-dependent cellular phagocytosis (ADCP) or complement-dependent cytotoxicity (CDC).

41. The method of any of claims 26-40, wherein the conjugate comprises an IgG1 Fc region or an IgG1 isotype, an IgG2 Fc region or an IgG2 isotype, IgG3 Fc region or an IgG3 isotype, or an IgG4 Fc region or an IgG4 isotype.

42. The method of any of claims 26-41, wherein the antibody or antibody-binding fragment comprises an IgG1 Fc region or an IgG1 isotype.

43. The method of claim 42, wherein the IgG1 Fc region is not enhanced for ADCC effector function.

44. The method of any of claims 26-43, wherein the antibody or antibody-binding fragment comprises an IgG2 Fc region or an IgG2 isotype.

45. The method of claim 44, wherein the IgG2 Fc region comprises a substitution that reduces or abrogates ADCC effector function.

46. The method of claim 45, wherein the substitution is a substitution of glutamine for the asparagine in the Fc region at the position corresponding to 297 according to EU numbering (N297Q).

47. The method of any of claims 25-46, further comprising administering an immune checkpoint inhibitor therapy subsequent to the administration of the conjugate.

48. The method of claim 47, wherein the immune checkpoint inhibitor therapy is administered 1 day, 2 days, 3 days, 4 days, 5 days, 6 days, 7 days, 1 week, 2 weeks or 3 weeks following administration of the conjugate.

49. The method of claim 47 or 48, wherein the immune checkpoint inhibitor therapy is administered 1 day, 2 days, 3 days, 4 days, 5 days, 6 days, 7 days, 1 week, 2 weeks or 3 weeks following the illumination.

50. The method of any of claims 47-49, wherein the immune checkpoint inhibitor therapy is administered more than once following administration of the conjugate.

51. The method of any of claims 47-50, wherein the immune checkpoint inhibitor therapy comprises a PD-1 inhibitor, a PD-L1 inhibitor or a CTLA-4 inhibitor.

52. The method of claim 51, wherein the PD-1 inhibitor is an anti-PD-1 antibody selected from pembrolizumab (MK-3475, KEYTRUDA; lambrolizumab), nivolumab (OPDIVO), cemiplimab (LIBTAYO), toripalimab (JS001), HX008, SG001, GLS-010, dostarlimab (TSR-042), tislelizumab (BGB-A317), cetrelimab (JNJ-63723283), pidilizumab (CT-011), genolimzumab (APL-501, GB226), BCD-100, cemiplimab (REGN2810), F520, sintilimab (IB1308), CS1003, LZM009, camrelizumab (SHR-1210), SCT-I10A, MGA012, AK105, PF-06801591, AMP-224, AB122, AMG 404, BI 754091, HLX10, JTX-4014, AMP-514 (MEDI0680), Sym021, MGD019, MGD013, AK104, XmAb20717, R07121661, CX-188, spartalizumab, BCD-217, HX009, IBI308, PDR001, REGN2810, TSR-042 (ANB011), or an antigen-binding fragment thereof or any combination thereof.

53. The method of any of claims 25-52, wherein the population of regulatory T cells (Tregs) in the tumor or the lesion or in the tumor microenvironment is reduced as a result of the method.

54. The method of any of claims 25-53, wherein the reduction or inhibition comprises one or more of less than 20% increase in tumor volume or tumor dimensions, or a reduction in tumor volume, tumor dimensions or tumor mass, or a reduction in number of tumor cells.

55. The method of any of claims 25-54, wherein the growth, volume or dimensions of the tumor or the lesion is inhibited or reduced to a greater degree as compared to a method that employs a conjugate comprising an antibody or antigen-binding fragment that specifically binds a CD25 and substantially blocks or interferes with IL-2 signaling.

56. The method of any of claims 25-55, wherein the method improves the survival of the subject.

57. The method of any of claims 25-56, wherein the subject comprises a second tumor or secondary population of tumor cells, and wherein the growth, volume or dimensions of the second tumor or secondary population of tumor cells is reduced or inhibited as a result of the method.

58. The method of claim 57, wherein the second tumor or secondary population of tumor cells has not been illuminated.
